(19) United States
(12) Patent Application Publication
(10) Pub. No.: US 2007/0101444 A1
(43) Pub. Date: May 3, 2007

(54) Ion channel

(76) Inventors: Nicola Brice, Cambridge (GB); John Dixon, Cambridge (GB); Sophie Messager, Cambridge (GB); Dirk Zahn, Cambridge (GB)

Correspondence Address: FROMMER LAWRENCE & HAUG, 745 FIFTH AVENUE- 10TH FL., NEW YORK, NY, 10151, US

(21) Appl. No.: 11/529,048

(22) Filed: Sep. 28, 2006

Related U.S. Application Data
(63) Continuation-in-part of application No. PCT/GB05/01151, filed on Mar. 30, 2005.
(60) Provisional application No. 60/561,123, filed on Apr. 8, 2004.

(30) Foreign Application Priority Data
Mar. 30, 2004 (GB) 0407175.9

(51) Int. Cl. A01K 67/027

(52) U.S. Cl. 800/3; 800/18

(57) ABSTRACT

Provided are screens for identifying compounds capable of binding to TrpM8 polypeptide. Theses compounds can be antagonists of TrpM8 polypeptide activity. Also provided is a transgenic non-human animal having a functionally disrupted endogenous TrpM8 gene.

### CROSS REFERENCE TO RELATED APPLICATIONS

This application is a continuation-in-part of International application no. PCT/GB2005/001151, filed Mar. 30, 2005, published as WO 2005/094569 on Oct. 13, 2005, and claiming priority to GB 0407175.9, filed Mar. 30, 2004, and to U.S. 60/561,123, filed Apr. 8, 2004.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

This invention relates to newly identified nucleic acids, polypeptides encoded by them and to their production and use. More particularly, the nucleic acids and polypeptides of the present invention relate to an ion channel, hereinafter referred to as “TrpM8” or “TipM8 ion channel”. The invention also relates to inhibiting or activating the action of such nucleic acids and polypeptides.

### BACKGROUND OF THE INVENTION

The mammalian nervous system constantly evaluates internal and environmental temperatures to maintain homeostasis and to avoid thermal extremes. Transient receptor potential (TRP) channels form cationic channels activated by diverse factors including mechanical stimuli, changes in osmolarity, pH and temperature as well as the exogenous irritant, capsaicin. These specialised sensory receptors are found in the Dorsal Root Ganglia (DRG).

Several members of the transient receptor potential (TRP) family of ion channels have been implicated as transducers of thermal stimuli, including TRPV1 and TRPV2, which are activated by heat, and TRPM8, which is activated by cold.

A cold- and menthol-sensitive receptor (CMRI) derived from rat has been cloned recently [McKemy D. D., Neuhausser W. M., and Julius, D.: Identification of a cold receptor reveals a general role for TRP channels in thermosensation. Nature 416:5258, 2002]. This receptor is an excitatory ion channel expressed by small-diameter neurons in trigeminal and, dorsal root ganglia. This channel receptor is activated by cold temperature (8-28 degrees C.) and menthol as a chemical agonist of a thermally responsive receptor, eliciting the same sensation of cool feeling. CNIR1 belongs in a member of the transient receptor potential (TRP) channel subfamily, which is similar to other thermoreceptors, −VR1 and VRL1, responding with a noxious heat and transfer the sensory information to the spinal cord and brain [Nagy L, Rang H. Noxious heat activates all capsaicin-sensitive and also a sub-population of capsaicin insensitive dorsal root ganglion neurons. Neuroscience 88:995-997, 1999] [Cesare P., McNaughton P.: A novel heat-activated current in nociceptive neurons and its sensitization by bradykinin. Proc. Natl. Acad. Sci. U.S.A. 93:15435-15439 1996].

Recent electrophysiological studies of cultured dorsal root and trigeminal ganglion neurons have suggested that multiple ionic mechanisms underlie the peripheral detection of cold temperatures. Several candidate “cold receptors,” all of them ion channel proteins, have been implicated in this process. One candidate is TRPM8, a nonselective cationic channel expressed in a subpopulation of sensory neurons that is activated both by decreases in temperature and the cooling compound menthol. Combined fluorometric calcium imaging of cultured rat trigeminal neurons with single-cell RT-PCR has demonstrated that there are distinct subpopulations of cold responsive neurons and that TRPM8 likely contributes to cold transduction in one of them. TRPM8 is preferentially expressed within a subset of rapidly responsive, low-threshold (less than 30 degrees C.), cold-sensitive neurons.

The function of mouse TRPM8 was characterized as an ion channel gated by cold stimuli and menthol, and its expression was limited in a subpopulation of the pain- and temperature-sensing DRG neurons [Peiet A. M., Moqrich A., Hergarden A. C., Reeve A. J., Andersson D. A., Story G. M., Earley T. J., Dragoni I., McIntyre P., Bevan S., Patapoutian A.: A TRP Channel that Senses Cold Stimuli and Menthol. Cell 108:705-715, 2002].

We have now found that mice TrpM8 ion channel is also triggered by noxious heat stimuli and therefore may have an effect in pain. We also demonstrate the involvement of TrpM8 ion channel in stress and anxiety.

### SUMMARY OF THE INVENTION

According to a first aspect of the present invention, we provide a transgenic non-human animal having a functionally disrupted endogenous TrpM8 gene, in which the TrpM8 gene comprises a nucleic acid sequence shown in SEQ ID NO: 1, SEQ ID NO: 2 or SEQ ID NO: 4 or a sequence having at least 70% sequence identity thereto.

Preferably, the transgenic non-human animal has a deletion in a TrpM8 gene or a portion thereof.

Preferably, it displays any one or combination of the following phenotypes: (a) decreased sensitivity to pain, preferably as measured in a tail-flick test; (b) decreased stress, preferably as measured in an open field test; (c) decreased blood plasma coiticosterone levels; as compared to a wild-type animal.

We further provide a transgenic non-human animal in which at least a portion or the whole of the TrpM8 gene of the animal is replaced with a sequence from the TrpM8 gene of another animal, preferably another species, more preferably a human.

Preferably, the transgenic non-human animal is a mouse.

Preferably, the transgenic non-human animal comprises a functionally disrupted TrpM8 gene, preferably a deletion in a TrpM8 gene, in which the TrpM8 gene comprises a nucleic acid sequence shown in SEQ ID NO: 4 or a sequence having at least 70% sequence identity thereto.

There is provided, according to a second aspect of the present invention, an isolated cell or tissue from a non-human transgenic animal according to the first aspect of the invention.

We provide, according to a third aspect of the present invention, a cell having a functionally disrupted endogenous TrpM8 gene, in which the TrpM8 gene comprises a nucleic acid sequence shown in SEQ ID NO: 1, SEQ ID NO: 2 or SEQ ID NO: 4, or a sequence having at least 70% sequence identity thereto.

As a fourth aspect of the present invention, there is provided use of a transgenic non-human animal, a cell or tissue or a cell according to, each as set out above, as a model for pain or stress.

We provide, according to a fifth aspect of the present invention, use of a transgenic non-human animal, a cell or tissue or a cell according to, each as set out above, as a model for a TrpM8 associated disease.

In a sixth aspect of the present invention, there is provided use of a non-human transgenic animal, an isolated cell or tissue thereof, or a cell, each as described, in a method of identifying an agonist or antagonist of a TrpM8 polypeptide comprising an amino acid sequence shown in SEQ ID NO: 3 or SEQ ID NO: 5 or a sequence having at least 70% sequence identity thereto.

According to a seventh aspect of the present invention, we provide a method of identifying an agonist or antagonist of a TrpM8 polypeptide having an amino acid sequence shown in SEQ ID NO: 3 or SEQ ID NO: 5 or a sequence having at least 70% sequence identity thereto, the method comprising administering a candidate compound to an animal, preferably a wild type animal or a transgenic non-human animal as set out above, and measuring a change in any of the following phenotypes: (a) sensitivity to pain, preferably as measured in a tail-flick test; (b) stress, preferably as measured in an open field test, and (c) blood plasma corticosterone levels.

Preferably, the method identifies an agonist of TrpM8 polypeptide according to by identifying a candidate compound capable of causing the animal to display a increase in any of the phenotypes (a)-(c).

Alternatively, or in addition, the method identifies an antagonist of TrpM8 polypeptide by identifying a candidate compound capable of causing the animal to display any of the phenotypes (a)-(c) or a decrease in such a phenotype.

We provide, according to an eighth aspect of the invention, a method of identifying an agonist or antagonist of a TrpM8 polypeptide having an amino acid sequence shown in SEQ ID NO: 3 or SEQ ID NO: 5 or a sequence having at least 70% sequence identity thereto, the method comprising exposing a candidate compound to a cell or tissue, preferably wild type cell or tissue or a cell or tissue or a cell as described above and measuring a change in conductance or intracellular calcium concentration of the cell or a cell of the tissue.

Preferably, the method identifies an agonist of TrpM8 polypeptide by identifying a candidate compound capable of increasing conductance or intracellular calcium concentration of the cell.

Alternatively, or in addition, the method of identifies an antagonist of TrpM8 polypeptide by identifying a candidate compound capable of decreasing conductance or intracellular calcium concentration of the cell.

There is provided, in accordance with a ninth aspect of the present invention, a method of identifying a compound suitable for the treatment or alleviation of pain or stress, preferably a TrpM8 associated disease, the method comprising exposing a TrpM8 polypeptide comprising an amino acid sequence shown in SEQ ID NO: 3 or SEQ ID NO: 5 or a sequence having at least 70% sequence identity thereto to a candidate compound, and determining whether the candidate compound is an antagonist or antagonist of the TrpM8 polypeptide.

As a tenth aspect of the invention, we provide use of a TrpM8 polynucleotide comprising a nucleic acid sequence shown in SEQ ID NO: 1, SEQ ID NO: 2 or SEQ ID NO: 4 or a sequence having at least 70% sequence identity thereto, for the identification of an agonist or antagonist thereof for the treatment of pain or stress, preferably a TrpM8 associated disease.

We provide, according to an eleventh aspect of the invention, use of a TrpM8 polypeptide comprising an amino acid sequence shown in SEQ ID NO: 3 or SEQ ID NO: 5 or a sequence having at least 70% sequence identity thereto, for the identification of an agonist or antagonist thereof for the treatment of pain or stress, preferably a TrpM8 associated disease.

According to a twelfth aspect of the present invention, we provide an antagonist of a TrpM8 polypeptide having an amino acid sequence shown in SEQ ID NO: 3 or SEQ ID NO: 5 or a sequence having at least 70% sequence identity thereto for use in a method of treatment of pain or stress, preferably a TrpM8 associated disease, in an individual.

There is provided, according to a thirteenth aspect of the present invention, use of an antagonist of a TrpM8 polypeptide having an amino acid sequence shown in SEQ ID NO: 3 or SEQ ID NO: 5 or a sequence having at least 70% sequence identity thereto for the preparation of a pharmaceutical composition for the treatment of pain or stress, preferably a TrpM8 associated disease, in an individual.

We provide, according to a fourteenth aspect of the present invention, a method of treating an individual suffering from pain or stress, preferably suffering from a TrpM8 associated disease, the method comprising administering an antagonist of TrpM8 to the individual.

According to a fifteenth aspect of the present invention, we provide a method of diagnosis of pain or stress, preferably a TrpM8 associated disease, in an individual, the method comprising detecting a change in expression, level or activity of TrpM8 in the individual or a cell or tissue thereof.

Preferably, the TrpM8 associated disease is selected from the group consisting of: Pain, cancer, inflammatory, inflammatory bowel disease, thermal hyperalgesia, visceral pain, migraine, post herpatic neuralgia, diabetic neuralgia, trigeminal neuralgia, post operative pain, osteoarthritis, rhuematoid arthritis, acute pain, chronic pain, cutaneous pain, somatic pain, visceral pain, referred pain, including myocardial ischaemia, phantom pain, neuropathic pain (neuralgia), pain arising from injuries, diseases, headaches, migraines, cancer pain, pain arising from neurological disorders such as Parkinson's disease, pain arising from spine and peripheral nerve surgery, brain tumors, traumatic brain injury (TBI), spinal cord trauma, chronic pain syndromes, chronic fatigue syndrome, neuralgias such as trigeminal neuralgia, glossopharyngeal neuralgia, postherpetic neuralgia and causalgia, pain arising from lupus, sarcoidosis, arachnoiditis, arthritis, rheumatic disease, period pain, back pain, lower back pain, joint pain, abdominal pain, chest pain, labour pain, musculoskeletal and skin diseases, diabetes, head trauma, and fibromyalgia, breast, prostate, colon, lung, ovarian, and bone cancer, social anxiety, post traumatic stress disorder, phobias, social phobia, special phobias, panic disorder, obsessive compulsive disorder, acute stress, disorder, separation anxiety disorder, generalised anxiety disorder, major depression, dysthymia, bipolar disorder, seasonal affective disorder, post natal depression, manic depression, bipolar depression.

According to a sixteenth aspect of the present invention, we provide a TrpM8 polypeptide comprising the amino acid sequence shown in SEQ ID NO. 3 or SEQ ID NO: 5, or a homologue, variant or derivative thereof having at least 70% sequence identity thereto.

We provide, according to a seventeenth aspect of the present invention, a nucleic acid encoding such a polypeptide.

Preferably, such a nucleic acid comprises the nucleic acid sequence shown in SEQ ID NO: 1, SEQ ID NO: 2 or SEQ ID NO: 4, or a homologue, variant or derivative thereof having at least 70% sequence identity thereto.

### BRIEF DESCRIPTION OF THE DRAWINGS

FIG. 1 is a diagram showing the knockout vector.

FIG. 2 shows the gene expression results from the RT-PCR testing.

FIG. 3 is a graph showing the results of the tail flick studies for knockout mice (mutant) compared with wild-type mice (wt).

FIGS. 4A-C are graphs showing the results of the open field test for knockout mice (mutant, −/−), compared with wild-type mice (wt, +/+). FIG. 4A shows the permance time in the central zone. FIG. 4B shows the distance moved in the central zone. FIG. 4C shows the total distance moved.

FIG. 5 is a graph showing the results of the assay for blood plasma corticosterone levels, for female knockout mice (mutant, white bar), compared to female wild-type mice (black bar) at 3 months.

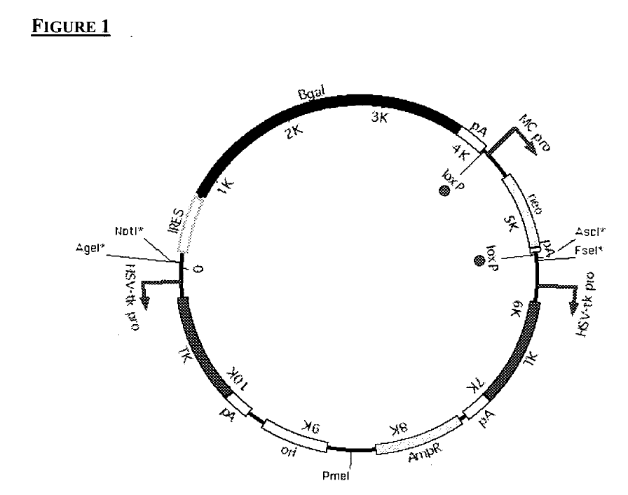

### SEQUENCES

SEQ ID NO: 1 shows the cDNA sequence of human TrpM8.

SEQ ID NO: 2 shows an open reading frame derived from SEQ ID NO: 1.

SEQ ID NO: 3 shows the amino acid sequence of human TrpM8.

SEQ ID NO: 4 shows the open reading frame of a cDNA for Mouse TrpM8.

SEQ ID NO: 5 shows the amino acid sequence of Mouse TrpM8.

SEQ ID NOs. 6-18 show the genotyping primers used to construct the knockout plamsid.

SEQ ID NO: 19 shows the knockout plasmid sequence.

### DETAILED DESCRIPTION

TRPM8 Ion Channel

We describe an ion channel, in particular, to TrpM8 ion channel, as well as homologues, variants or derivatives thereof, as well as their uses in treatment and diagnosis of diseases, including TrpM8 associated diseases.

TrpM8 is structurally related to other proteins of the ion channel family, as shown by the results of sequencing the amplified cDNA products encoding human TrpM8. The cDNA sequence of SEQ ID NO: 1 contains an open reading flame (SEQ ID NO: 2, nucleotide numbers 41 to 3352) encoding a polypeptide of 1104 amino acids shown in SEQ ID NO: 3. Human TrpM8 is found to map to Homo sapiens chromosome 2q37.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press inc., Boca Raton, Fl., 1988, ISBN 0-8493-6078-1, John D. Pound (ed); “Immunochemical Protocols, vol 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3; and The Merck Manual of Diagnosis and Therapy (17th Edition, Beers, M. H., and Berkow, R, Eds, ISBN: 0911910107, John Wiley & Sons). Each of these general texts is incorporated herein by reference.

Identities and Similarities to TrpM8

Analysis of the TrpM8 polypeptide (SEQ ID NO: 3) using the HMM structural prediction software of pfam (available at the pfam website maintained by the Sanger Institute) confirms that TrpM8 peptide is an ion channel.

The mouse homologue of the human TrpM8 ion channel has been cloned, and its nucleic acid sequence and amino acid sequence are shown as SEQ ID NO: 4 and SEQ ID NO: 5 respectively. The mouse TrpM8 ion channel cDNA of SEQ ID NO: 4 shows a high degree of identity with the human TrpM8 ion channel (SEQ ID NO: 2) sequence, while the amino acid sequence (SEQ ID NO: 5) of mouse TrpM8 ion channel shows a high degree of identity and similarity with human TrpM8 ion channel (SEQ ID NO: 3). Human and mouse TrpM8 ion channel are therefore members of a large family of ion channels.

Expression Profile of TrpM8

Polymerase chain reaction (PCR) amplification of TrpM8 cDNA detects expression of TrpM8 to varying abundance in the prostate (+++), Liver (+++), Muscle (+), Testis (++), and Ovary (+).

Using TrpM8 cDNA of SEQ ID NO: 1 to search the human EST data sources by BLASTN, identities are found in cDNA libraries. This indicates that TrpM8 is expressed in these normal or abnormal tissues.

| | |
|---|---|
| BE274448.1 | human skin |
| BE390627 | human uterus |
| AW295430 | human prostate |
| BG567490 | human liver |
| BE791173 | human small cell carcinoma, lung, MGC3 |
| BE408880 | human placenta, choriocarcinoma |
| BF244389 | human brain, glioblastoma |
| BG565397 | human liver |
| BE207083 | human small cell carcinoma, lung, MGC3 |
| BE274448 | human skin, melanotic melanoma |
| BE390627 | human uterus, endometrium, adenocarcinoma cell line. |

Accordingly, the TrpM8 polypeptides, nucleic acids, probes, antibodies, expression vectors and ligands are useful for detection, diagnosis, treatment and other assays for diseases associated with over-, under- and abnoimal expression of TrpM8 ion channel in these and other tissues. Such diseases may include the TrpM8 associated diseases set out below.

TrpM8 Ion Channel Associated Disease

According to the methods and compositions described here, TrpM8 ion channel is useful for treating and diagnosing a range of diseases, described in detail below. These diseases are referred to for convenience as TrpM8 associated diseases.

We demonstrate here that human TrpM8 maps to Homo sapiens chromosome 2q37. Accordingly, in a specific embodiment, TrpM8 ion channel may be used to treat or diagnose a disease which maps to this locus, chromosomal band, region, arm or the same chromosome. Known diseases which have been determined as being linked to the same locus, chromosomal band, region, aim or chromosome as the chromosomal location of TrpM8 ion channel (i.e., Homo sapiens chromosome 2q37) include prostate cancer where the ion channel has been found to be upregulated.

Knockout mice deficient in TrpM8 display a range of phenotypes, as demonstrated in the Examples.

In particular, we disclose at Example 4 below that TrpM8 deficient mice are hypoalgesic, i.e., less sensitive to pain. TrpM8 and modulators of TrpM8 activity, including in particular antagonists of TrpM8, may be used to treat or alleviate diseases or syndromes in which pain is a feature. Specifically, activity or expression of TrpM8 may be down-regulated in an individual, for example by administration of antagonists or blockers of TrpM8, for analgesia/in order to reduce pain. We therefore disclose the identification of antagonists of TrpM8 for use as analgesics.

Accordingly, according to a preferred embodiment, the methods and compositions described here, including TrpM8 ion channel and its modulators and antagonists may be used to diagnose or treat or relieve, by any means as described in this document, pain and cancer. Particularly, pain includes neuropathic, inflammatory, inflammatory bowel disease, thermal hyperalgesia, visceral pain, migraine, post herpatic neuralgia, diabetic neuralgia, trigeminal neuralgia, post operative pain, osteoarthritis, rhuematoid arthritis. Also included are acute pain, chronic pain, cutaneous pain, somatic pain, visceral pain, referred pain, including myocardial ischaemia, phantom pain and neuropathic pain (neuralgia). The definition of pain includes, but is not limited to pain arising from injuries, diseases, headaches, migraines, cancer pain, pain arising from neurological disorders such as Parkinson's disease, pain arising from spine and peripheral nerve surgery, brain tumors, traumatic brain injury (TBI), spinal cord trauma, chronic pain syndromes, chronic fatigue syndrome, neuralgias such as trigeminal neuralgia, glossopharyngeal neuralgia, postherpetic neuralgia and causalgia, pain arising from lupus, sarcoidosis, arachnoiditis, arthnitis, rheumatic disease, period pain, back pain, lower back pain, joint pain, abdominal pain, chest pain, labour pain, musculoskeletal and skin diseases, diabetes, head trauma, and fibromyalgia. Particularly, cancer includes breast, prostate, colon, lung, ovarian, and bone cancer.

Example 5 describes an Open Field test, in which TrpM8 knockout mice are shown to be less anxious than their wild type counterparts. Furthermore, blood plasma levels of corticosterone, an indicator of stress and anxiety, are seen to be lower in TrpM8 knockout mice than corresponding wild type mice (Example 6). A deficit of TrpM8 activity is therefore correlated with a decrease in stress.

We therefore disclose a method of lowering stress or anxiety or both in an individual, the method comprising decreasing the level or activity of TrpM8 in that individual. As noted elsewhere, this can be achieved by down-regulating the expression of TrpM8, or by use of antagonists to TrpM8.

TrpM8 and modulators of TrpM8 activity, including in particular antagonists of TrpM8, may be used to treat or alleviate diseases or syndromes in which stress and anxiety feature. Such diseases include social anxiety, post traumatic stress disorder, phobias, social phobia, special phobias, panic disorder, obsessive compulsive disorder, acute stress, disorder, separation anxiety disorder, generalised anxiety disorder, major depression, dysthymia, bipolar disorder, seasonal affective disorder, post natal depression, manic depression, bipolar depression.

In a preferred embodiment, the TrpM8 associated disease comprises a disease in which stress or anxiety is a symptom. In a highly preferred embodiment, the TrpM8 disease comprises the above list of anxiety and stress related diseases.

As noted above, TrpM8 ion channel may be used to diagnose and/or treat any of these specific diseases using any of the methods and compositions described here.

In particular, we specifically envisage the use of nucleic acids, vectors comprising TrpM8 ion channel nucleic acids, polypeptides, including homologues, variants or derivatives thereof, pharmaceutical compositions, host cells, and transgenic animals comprising TrpM8 ion channel nucleic acids and/or polypeptides, for the treatment or diagnosis of the specific diseases listed above. Furthermore, we envisage the use of compounds capable of interacting with or binding to TrpM8 ion channel, preferably antagonists of TrpM8, preferably a compound capable decreasing the conductance of the channel, antibodies against TrpM8 ion channel, as well as methods of making or identifying these, in diagnosis or treatment of the specific diseases mentioned above. In particular, we include the use of any of these compounds, compositions, molecules, etc, in the production of vaccines for treatment or prevention of the specific diseases. We also disclose diagnostic kits for the detection of the specific diseases in an individual.

Methods of linkage mapping to identify such or further specific diseases treatable or diagnosable by use of TrpM8 ion channel are known in the art, and are also described elsewhere in this document.

Anxiety and Stress

Anxiety and stress, as well as disorders in which these are manifested, including TrpM8 associated diseases, are well known in the art. A summary description follows:

Anxiety and stress are also referred to as feeling uptight, tension, jitters, and apprehension. Stress can come from any situation or thought that makes an individual feel frustrated, angry, or anxious. What is stressful to one person is not necessarily stressful to another.

Anxiety is a feeling of apprehension or fear. The source of this uneasiness is not always known or recognized, which can add to the distress the individual feels.

Stress is a normal part of life. In small quantities, stress is may be beneficial—it can motivate an individual and him to be more productive. However, too much stress, or a strong response to stress, is harmful. It can set the individual up for general pool health as well as specific physical or psychological illnesses like infection, heart disease, or depression. Persistent and unrelenting stress often leads to anxiety and unhealthy behaviors like overeating and abuse of alcohol or drugs.

Emotional states like grief or depression and health conditions like an overactive thyroid, low blood sugar, or heart attack can also cause stress.

Anxiety is often accompanied by physical symptoms, including: twitching or trembling, muscle tension, headaches, sweating, dry mouth, difficulty swallowing, abdominal pain (this may be the only symptom of stress, especially in a child)

Sometimes other symptoms accompany anxiety: dizziness, rapid or irregular heart rate, rapid breathing, diarrhoea or frequent need to urinate, fatigue, irritability, including loss of temper, sleeping difficulties and nightmares, decreased concentration and sexual problems.

TrpM8 and its modulators may be used to treat or alleviate any of these symptoms.

Anxiety disorders are a group of psychiatric conditions that involve excessive anxiety. They include generalized anxiety disorder, specific phobias, obsessive-compulsive disorder, and social phobia. See also TrpM8 associated diseases set out above.

Certain drugs, both recreational and medicinal, can lead to symptoms of anxiety due to either side effects or withdrawal from the drug. Such drugs include caffeine, alcohol, nicotine, cold remedies, decongestants, bronchodilators for asthma, tricyclic antidepressants, cocaine, amphetamines, diet pills, ADHD medications, and thyroid medications. We disclose the use of TrpM8 and its modulators in combination with such drugs to alleviate their stress and/or anxiety inducing effects.

A poor diet can also contribute to stress or anxiety—for example, low levels of vitamin B12. Performance anxiety is related to specific situations, like taking a test or making a presentation in public. Post-traumatic stress disorder (PTSD) is a stress disorder that develops after a traumatic event like war, physical or sexual assault, or a natural disaster.

In very rare cases, a tumor of the adrenal gland (pheochromocytoma) may be the cause of anxiety. This happens because of an overproduction of hormones responsible for the feelings and symptoms of anxiety.

(Adapted from Medline Plus, available at the National Library of Medicine website.

Pain

Acute Pain

Acute pain is defined as short-term pain or pain with an easily identifiable cause. Acute pain is the body's warning of present damage to tissue or disease. It is often fast and sharp followed by aching pain. Acute pain is centralized in one area before becoming somewhat spread out.

Chronic Pain

Chronic pain is medically defined as pain that has lasted 6 months or longer. This constant or intermittent pain has often outlived its purpose, as it does not help the body to prevent injury. It is often more difficult to treat than acute pain. Expert care is generally necessary to treat any pain that has become chronic. When opioids are used for prolonged periods drug tolerance, chemical dependency and even psychological addiction may occur. While drug tolerance and chemical dependency are common among opioid users, psychological addiction is rare.

The experience of physiological pain can be grouped into four categories according to the source and related nociceptors (pain detecting nerves).

Cutaneous Pain

Cutaneous pain is caused by injury to the skin or superficial tissues. Cutaneous nociceptors terminate just below the skin, and due to the high concentration of nerve endings, produce a well-defined, localised pain of short duration. Example injuries that produce cutaneous pain include paper cuts, minor (first degree) burns and lacerations.

Somatic Pain

Somatic pain originates from ligaments, tendons, bones, blood vessels, and even nerves themselves, and are detected with somatic nociceptors. The scarcity of pain receptors in these areas produces a dull, poorly-localised pain of longer duration than cutaneous pain; examples include sprained ankle and broken bones.

Visceral Pain

Visceral pain originates from body organs visceral nociceptors are located within body organs and internal cavities. The even greater scarcity of nociceptors in these areas produces a pain usually more aching and of a longer duration than somatic pain. Visceral pain is extremely difficult to localise, and several injuries to visceral tissue exhibit “referred” pain, where the sensation is localised to an area completely unrelated to the site of injury. Myocardial ischaemia (the loss of blood flow to a part of the heart muscle tissue) is possibly the best known example of referred pain; the sensation can occur in the upper chest as a restricted feeling, or as an ache in the left shoulder, arm or even hand.

Other Types of Pain

Phantom limb pain is the sensation of pain from a limb that one no longer has or no longer gets physical signals from—an experience almost universally reported by amputees and quadriplegics. Neuropathic pain (“neuralgia”) can occur as a result of injury or disease to the nerve tissue itself. This can disrupt the ability of the sensory nerves to transmit correct information to the thalamus, and hence the brain interprets painful stimuli even though there is no obvious or documented physiological cause for the pain.

Trigeminal neuralgia (“tic douloureux”) refers to pain caused by injury or damage to the trigeminal nerve. The Trigeminal nerve has 3 branches: V1 gives sensation to the area of the forehead and eye and V2 gives sensation to the nose and face and V3 gives sensation to the jaw and chin area. Each side of the face has a trigeminal nerve that gives sensation. The one-sided pain of trigeminal neuralgia may extend through the cheek, mouth, nose and/or jaw muscles. Trigeminal neuralgia generally affects older people, although younger people or those with multiple sclerosis may also experience trigeminal neuralgia.

The primary symptom of trigeminal neuralgia is pain in either the forehead, cheek, chin or jawline. Severe cases may involve all three areas or both left and right sides. Pain episodes are severe, spastic and short, and are described as similar to what would be felt as electrical shock. The pain can be triggered by common daily activities such as brushing the teeth, talking, chewing, drinking, shaving or even kissing. The frequency of the pain episodes increases over time, becoming more disruptive and disabling.

Glossopharyngeal neuralgia is a clinical entity characterized by bursts of pain in the sensory distribution of the ninth cranial nerve. Except for the location of the pain and the stimulus for the pain the attacks are identical to trigeminal neuralgia. The typical pain is a severe lancinating, repetitive series of electrical-like stabs in the region of the tonsils or the back of the tongue, on one side. In addition, the pain may radiate to or originate in the ear.

The sensory stimulus which induces the pain is swallowing, and during severe attacks the patient may sit motionless, head flexed forward, allowing saliva to freely drool from the mouth. Cardiac arrest, syncope (fainting), and seizures have been associated with attacks of glossopharyngeal neuralgia. The cause of glossopharyngeal neuralgia in most cases is unknown. However, a certain number of cases have been ascribed to tumors, compression of the ninth nerve by the vertebral artery, and vascular malformations.

Postherpetic neuralgia refers to chronic pain continuing after an infection of herpes zoster virus. Herpes zoster, also known as shingles, is a recurrent infection of varicella-zoster (chickenpox) viral infection. The virus lies dormant within nerves until the patient's immunity wanes. The acute lesion of shingles causes pain which usually goes away. However, in a number of patients the pain continues chronically—postherpetic neuralgia.

The symptoms of herpes zoster include a lancinating, deep, continuous pain: the pain is in the thoracic region 65% and the face 20%. When the face is involved the virus shows a predilection for the ophthalmic division of the trigeminal nerve (top of the face above the eyebrows). The pain usually resolves spontaneously in 2 to 4 weeks. However, a few patients will have persistent pain. The pain is in the region of the previous rash and is exacerbated by gently stroking the affected skin and is relieved by applying pressure to the area. The rubbing of clothing is often very painful. This continuing pain is called postherpetic neuralgia. There is a higher incidence of postherpetic neuralgia in cases of herpes zoster involving the face.

Causalgia is a rare syndrome that follows partial peripheral nerve injuries. It is characterized by a triad of burning pain, autonomic dysfunction and trophic changes. Severe cases are called major causalgia. Minor causalgia describes less severe forms, similar to reflex sympathetic dystrophy (RSD). RSD has predominant muscular and joint symptoms, with osteoporosis being common on x-ray.

Causalgia is caused by peripheral nerve injuries, usually brachial plexus injuries. Denervation causes hypersensitivity resulting in increased pain and increased norepinephrine release causes the sympathetic findings. Symptoms include Pain: usually burning, and prominent in hand or foot. Onset in the majority is within 24 hours of injury. The median, ulnar and sciatic nerves are the most commonly involved. Almost any sensory stimulation worsens the pain. Vascular changes: Either increased blood by vasodilatation (warm and pink) or decreased blood by vasoconstriction (cold, mottled blue). Trophic changes: dry/scaly skin, stiff joints, tapering fingers, ridged uncut nails, either long/coarse hair or loss of hair, sweating alteration.

TrpM8 Ion Channel Polypeptides

As used here, the term “TrpM8 ion channel polypeptide” is intended to refer to a polypeptide comprising the amino acid sequence shown in SEQ ID NO: 3 or SEQ ID NO: 5, or a homologue, variant or derivative thereof. Preferably, the polypeptide comprises or is a homologue, variant or derivative of the sequence shown in SEQ ID NO: 3.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et aL, “Protein Synthesis: Posttranslational Modifications and Aging”, Ann NY Acad Sci (1992) 663:48-62.

The terms “variant”, “homologue”, “derivative” or “fragment” as used in this document include any substitution of, variation of, modification of, replacement of, deletion of or addition of one (or more) amino acid from or to a sequence. Unless the context admits otherwise, references to “TrpM8” and “TrpM8 ion channel” include references to such variants, homologues, derivatives and fragments of TrpM8.

Preferably, as applied to TrpM8, the resultant amino acid sequence has ion channel activity, more preferably having at least the same activity of the TrpM8 ion channel shown as SEQ ID NO: 3 or SEQ ID NO: 5. In particular, the term “homologue” covers identity with respect to structure and/or function providing the resultant amino acid sequence has ion channel activity. With respect to sequence identity (i.e. similarity), preferably there is at least 70%, more preferably at least 75%, more preferably at least 85%, even more preferably at least 90% sequence identity. More preferably there is at least 92%, at least 95%, or at least 98%, sequence identity. These terms also encompass polypeptides derived from amino acids which are allelic variations of the TrpM8 ion channel nucleic acid sequence.

Where reference is made to the “channel activity” or “biological activity” of an ion channel such as TrpM8 ion channel, these terms are intended to refer to the metabolic or physiological function of the TrpM8 ion channel, including similar activities or improved activities or these activities with decreased undesirable side effects. Also included are antigenic and immunogenic activities of the TrpM8 ion channel. Examples of ion channel activity, and methods of assaying and quantifying these activities, are known in the art, and are described in detail elsewhere in this document.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

TrpM8 polypeptides as described here may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

TrpM8 polypeptides may further comprise heterologous amino acid sequences, typically at the N-terminus or C-terminus, preferably the N-terminus. Heterologous sequences may include sequences that affect intra or extracellular protein targeting (such as leader sequences). Heterologous sequences may also include sequences that increase the immunogenicity of the polypeptide and/or which facilitate identification, extraction and/or purification of the polypeptides. Another heterologous sequence that is particularly preferred is a polyamino acid sequence such as polyhistidine which is preferably N-terminal. A polyhistidine sequence of at least 10 amino acids, preferably at least 17 amino acids but fewer than 50 amino acids is especially preferred.

The TrpM8 ion channel polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

TrpM8 polypeptides are advantageously made by recombinant means, using known techniques. However they may also be made by synthetic means using techniques well known to skilled persons such as solid phase synthesis. The polypeptides described here may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. Preferably the fusion protein will not hinder the function of the protein of interest sequence.

TrpM8 polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated,” as the term is employed herein.

It will however be understood that the TrpM8 ion channel protein may be mixed with carriers or diluents which will not interfere with the intended purpose of the protein and still be regarded as substantially isolated. A TrpM8 polypeptide may also be in a substantially purified form, in which case it will generally comprise the protein in a preparation in which more than 90%, for example, 95%, 98% or 99% of the protein in the preparation is a TrpM8 polypeptide.

We further describe peptides comprising a portion of a TrpM8 polypeptide. Thus, fragments of TrpM8 ion channel and its homologues, variants or derivatives are included. The peptides may be between 2 and 200 amino acids, preferably between 4 and 40 amino acids in length. The peptide may be derived from a TrpM8 polypeptide as disclosed here, for example by digestion with a suitable enzyme, such as trypsin. Alternatively the peptide, fragment, etc may be made by recombinant means, or synthesised synthetically.

The term “peptide” includes the various synthetic peptide variations known in the art, such as retroinverso D peptides. The peptide may be an antigenic determinant and/or a T-cell epitope. The peptide may be immunogenic in vivo. Preferably the peptide is capable of inducing neutralising antibodies in vivo.

By aligning TrpM8 ion channel sequences from different species, it is possible to determine which regions of the amino acid sequence are conserved between different species (“homologous regions”), and which regions vary between the different species (“heterologous regions”).

The TripM8 polypeptides may therefore comprise a sequence which corresponds to at least part of a homologous region. A homologous region shows a high degree of homology between at least two species. For example, the homologous region may show at least 70%, preferably at least 80%, more preferably at least 90%, even more preferably at least 92%, most preferably 95% or 98% identity at the amino acid level using the tests described above. Peptides which comprise a sequence which corresponds to a homologous region may be used in therapeutic strategies as explained in further detail below. Alternatively, the TrpM8 ion channel peptide may comprise a sequence which corresponds to at least part of a heterologous region. A heterologous region shows a low degree of homology between at least two species.

TrpM8 Ion Channel Polynucleotides and Nucleic Acids

We further disclose TrpM8 polynucleotides, TrpM8 nucleotides and TrpM8 nucleic acids, methods of production, uses of these, etc, as described in further detail elsewhere in this document.

The terms “TrpM8 polynucleotide”, “TrpM8 nucleotide” and “TrpM8 nucleic acid” may be used interchangeably, and are intended to refer to a polynucleotide/nucleic acid comprising a nucleic acid sequence as shown in SEQ ID NO: 1, SEQ ID NO: 2 or SEQ ID NO: 4, or a homologue, variant or derivative thereof. Preferably, the polynucleotide/nucleic acid comprises or is a homologue, variant or derivative of the nucleic acid sequence SEQ ID NO: 1 or SEQ ID NO: 2, most preferably, SEQ ID NO: 2.

These terms are also intended to include a nucleic acid sequence capable of encoding a polypeptides and/or a peptide, i.e., a TrpM8 polypeptide. Thus, TrpM8 ion channel polynucleotides and nucleic acids comprise a nucleotide sequence capable of encoding a polypeptide comprising the amino acid sequence shown in SEQ ID NO: 3 or SEQ ID NO: 5, or a homologue, variant or derivative thereof. Preferably, the TrpM8 ion channel polynucleotides and nucleic acids comprise a nucleotide sequence capable of encoding a polypeptide comprising the amino acid sequence shown in SEQ ID NO: 3, or a homologue, variant or derivative thereof.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

Preferably, the term “nucleotide sequence” means DNA.

The terms “variant,” “homologue,” “derivative” or “fragment” as used in this document include any substitution of, variation of, modification of, replacement of, deletion of or addition of one (or more) nucleic acids from or to the sequence of a TrpM8 nucleotide sequence. Unless the context admits otherwise, references to “TrpM8” and “TrpM8 ion channel” include references to such variants, homologues, derivatives and fragments of TrpM8.

Preferably, the resultant nucleotide sequence encodes a polypeptide having ion channel activity, preferably having at least the same activity of the ion channel shown as SEQ ID NO: 3 or SEQ ID NO: 5. Preferably, the term “homologue” is intended to cover identity with respect to structure and/or function such that the resultant nucleotide sequence encodes a polypeptide which has ion channel activity. With respect to sequence identity (i.e. similarity), preferably there is at least 70%, more preferably at least 75%, more preferably at least 85%, more preferably at least 90% sequence identity. More preferably there is at least 92%, even more preferably 95%, most preferably at least 98%, sequence identity. These terms also encompass allelic variations of the sequences.

Calculation of Sequence Homology

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by commercially available computer programs that can calculate percent identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid, pages 7-58 to 7-60).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at the website maintained by the National Center for Biotechnology Information, which is incorporated herein by reference. Search parameters can be defined and can be advantageously set over the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:.2264-68; Karlin and Altschul, 1993, Proc. Natl. Acad. Sci. USA 90:5873-7; see the National Center for Biotechnology Information website) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at http://www.ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified-; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see the National Center for Biotechnology Information website). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website. In some embodiments, no gap penalties are used when determining sequence identity.

Hybridisation

We further disclose nucleotide sequences that are capable of hybridising to the sequences presented herein, or any fragment or derivative thereof, or to the complement of any of the above.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Nucleotide sequences capable of selectively hybridising to the nucleotide sequences presented herein, or to their complement, will be generally at least 70%, preferably at least 75%, more preferably at least 85 or 90% or 92% and even more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences will comprise regions homologous to SEQ ID NOs: 1, 2 or 4, preferably at least 70%, 80%, 90% or 92% and more preferably at least 95% or 98% homologous to one of the sequences.

The term “selectively hybridizable” means that the nucleotide sequence used as a probe is used under conditions where a target nucleotide sequence is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Also included are nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred embodiment, we disclose nucleotide sequences that can hybridise to one or more of the TrpM8 ion channel nucleotide sequences under stringent conditions (e.g. 65° C. and 0.1×SSC {1×SSC=0.15 M NaCl, 0.015 M Na₃ Citrate pH 7.0). Where the nucleotide sequence is double-stranded, both strands of the duplex, either individually or in combination, are encompassed. Where the nucleotide sequence is single-stranded, it is to be understood that the complementary sequence of that nucleotide sequence is also of use.

We disclose nucleotide sequences that are capable of hybridising to the sequences that are complementary to the sequences presented herein, or any fragment or derivative thereof. Likewise, our disclosure encompasses nucleotide sequences that are complementary to sequences that are capable of hybridising to the sequence. These types of nucleotide sequences are examples of variant nucleotide sequences. In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (e.g. 65° C. and 0.1×SSC {1×SSC=0.15 M NaCl, 0.015 Na₃ citrate pH 7.0}) to the nucleotide sequences presented herein.

Cloning of TrpM8 Ion Channel and Homologues

The present disclosure encompasses nucleotide sequences that are complementary to the sequences presented here, or any fragment or derivative thereof. If the sequence is complementary to a fragment thereof then that sequence can be used as a probe to identify and clone similar ion channel sequences in other organisms etc.

This enables the cloning of TrpM8 ion channel, its homologues and other structurally or functionally related genes from human and other species such as mouse, pig, sheep, etc to be accomplished. Polynucleotides which are identical or sufficiently identical to a nucleotide sequence contained in SEQ ID NO: 1, SEQ ID NO: 2, SEQ ID NO: 4 or a fragment thereof, may be used as hybridization probes for cDNA and genomic DNA, to isolate partial or full-length cDNAs and genomic clones encoding TrpM8 ion channel from appropriate libraries. Such probes may also be used to isolate cDNA and genomic clones of other genes (including genes encoding homologues and orthologues from species other than human) that have sequence similarity, preferably high sequence similarity, to the TrpM8 ion channel gene. Hybridization screening, cloning and sequencing techniques are known to those of skill in the art and are described in, for example, Sambrook et al (supra).

Typically nucleotide sequences suitable for use as probes are 70% identical, preferably 80% identical, more preferably 90% identical, even more preferably 95% identical to that of the referent. The probes generally will comprise at least 15 nucleotides. Preferably, such probes will have at least 30 nucleotides and may have at least 50 nucleotides. Particularly preferred probes will range between 150 and 500 nucleotides, more particularly about 300 nucleotides.

In one embodiment, to obtain a polynucleotide encoding a TrpM8 polypeptide, including homologues and orthologues from species other than human, comprises the steps of screening an appropriate library under stringent hybridization conditions with a labelled probe having the SEQ ID NO: 1, SEQ ID NO: 2, SEQ ID NO: 4 or a fragment thereof and isolating partial or full-length cDNA and genomic clones containing said polynucleotide sequence. Such hybridization techniques are well known to those of skill in the art. Stringent hybridization conditions are as defined above or alternatively conditions under overnight incubation at 42 degrees C. in a solution comprising: 50% formamide, 5×SSC (150 mM NaCl, 15 mM trisodium citrate), 50 mM sodium phosphate (pH7.6), 5× Denhardt's solution, 10% dextran sulphate, and 20 microgram/ml denatured, sheared salmon sperm DNA, followed by washing the filters in 0.1×SSC at about 65 degrees C.

Functional Assay for TrpM8 Ion Channel

The cloned putative TrpM8 ion channel polynucleotides may be verified by sequence analysis or functional assays. In particular, the conductance of Xenopus oocytes tranfected as described above may be detected as a means of guaging and quantifying TrpM8 activity, useful for screening assays described below. Such a conductance assay is referred to for convenience as a “Functional Assay of TrpM8 (Conductance)”.

The putative TrpM8 ion channel or homologue may be assayed for activity in a “Functional Assay of TrpM8 (Conductance)” as follows. Capped RNA transcripts from linearized plasmid templates encoding the TrpM8 ion channel cDNAs are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in 96 mM NaCl, 2 mM KCl, 1 mMMgCl₂, 0.1 mMCaCl₂, 5 mM Hepes, pH 7.4 and supplemented with 200 mM mannitol at room temperature. OSM is 210 mOsm. The Xenopus system may also be used to screen known ligands and tissue/cell extracts for activating ligands, as described in further detail below.

Alternative functional assays include whole cell electrophysiology, fluorescence resonance energy transfer (FRET) analysis and FLIPR analysis.

Expression Assays for TrpM8 Ion Channel

In order to design useful therapeutics for treating TrpM8 ion channel associated diseases, it is useful to determine the expression profile of TrpM8 (whether wild-type or a particular mutant). Thus, methods known in the art may be used to determine the organs, tissues and cell types (as well as the developmental stages) in which TrpM8 is expressed. For example, traditional or “electronic” Northern blots may be conducted. Reverse-transcriptase PCR (RT-PCR) may also be employed to assay expression of the TrpM8 gene or mutant. More sensitive methods for determining the expression profile of TrpM8 include RNAse protection assays, as known in the art.

Northern analysis is a laboratory technique used to detect the presence of a transcript of a gene and involves the hybridization of a labelled nucleotide sequence to a membrane on which RNAs from a particular cell type or tissue have been bound. (Sambrook, supra, ch. 7 and Ausubel, F. M. et al. supra, ch. 4 and 16.) Analogous computer techniques (“electronic Northerns”) applying BLAST may be used to search for identical or related molecules in nucleotide databases such as GenBank or the LIFESEQ database (Incyte Pharmaceuticals). This type of analysis has advantages in that they may be faster than multiple membrane-based hybridizations. In addition, the sensitivity of the computer search can be modified to determine whether any particular match is categorized as exact or homologous.

The polynucleotides and polypeptides described here, including the probes described above, may be employed as research reagents and materials for discovery of treatments and diagnostics to animal and human disease, as explained in further detail elsewhere in this document.

Expression of TrpM8 Ion Channel Polypeptides

We further disclose a process for producing a TrpM8 polypeptide. The method comprises in general culturing a host cell comprising a nucleic acid encoding TrpM8 ion channel polypeptide, or a homologue, variant, or derivative thereof, under suitable conditions (i.e., conditions in which the TrpM8 ion channel polypeptide is expressed).

In order to express a biologically active TrpM8 ion channel, the nucleotide sequences encoding TrpM8 ion channel or homologues, variants, or derivatives thereof are inserted into appropriate expression vector, i.e., a vector which contains the necessary elements for the transcription and translation of the inserted coding sequence.

Methods which are well known to those skilled in the art are used to construct expression vectors containing sequences encoding TrpM8 ion channel and appropriate transcriptional and translational control elements. These methods include in vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized to contain and express sequences encoding TrpM8 ion channel. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The nature of the host cell employed does not matter.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (GIBCO/BRL), and the like, may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding TrpM8 ion channel, vectors based on SV40 or EBV may be used with an appropriate selectable marker.

In bacterial systems, a number of expression vectors may be selected depending upon the use intended for TrpM8 ion channel. For example, when large quantities of TrpM8 ion channel are needed for the induction of antibodies, vectors which direct high level expression of fusion proteins that are readily purified may be used. Such vectors include, but are not limited to, multifunctional E. coli cloning and expression vectors such as BLUESCRIPT (Stratagene), in which the sequence encoding TrpM8 ion channel may be ligated into the vector in frame with sequences for the amino-terminal Met and the subsequent 7 residues of β-0galactosidase so that a hybrid protein is produced, pIN vectors (Van Heeke, G. and S. M. Schuster (1989) J. Biol. Chem. 264:5503-5509), and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. For reviews, see Ausubel (supra) and Grant et al. (1987; Methods Enzymol. 153:516-544).

In cases where plant expression vectors are used, the expression of sequences encoding TrpM8 ion channel may be driven by any of a number of promoters. For example, viral promoters such as the 35S and 19S promoters of CaMV may be used alone or in combination with the omega leader sequence from TMV. (Takamatsu, N. (1987) EMBO J. 6:307-311.) Alternatively, plant promoters such as the small subunit of RUBISCO or heat shock promoters may be used. (Coruzzi, G. et al. (1984) EMBO J. 3:1671-1680; Broglie, R. et al. (1984) Science 224:838-843; and Winter, J. et al. (1991) Results Probl. Cell Differ. 17:85-105.). These constructs can be introduced into plant cells by direct DNA transformation or pathogen-mediated transfection. Such techniques are described in a number of generally available reviews. (See, for example, Hobbs, S. or Murry, L. E. in McGraw Hill Yearbook of Science and Technology (1992) McGraw Hill, New York, N.Y.; pp. 191-196.).

An insect system may also be used to express TrpM8 ion channel. For example, in one such system, Autographa californica nuclear polyhedrosis virus (AcNPV) is used as a vector to express foreign genes in Spodoptera frugiperda cells or in Trichoplusia larvae. The sequences encoding TrpM8 ion channel may be cloned into a non-essential region of the virus, such as the polyhedrin gene, and placed under control of the polyhedrin promoter. Successful insertion of TrpM8 ion channel will render the polyhedrin gene inactive and produce recombinant virus lacking coat protein. The recombinant viruses may then be used to infect, for example, S. frugiperda cells or Trichoplusia larvae in which TrpM8 ion channel may be expressed. (Engelhard, E. K. et al. (1994) Proc. Nat. Acad. Sci. 91:3224-3227.)

In mammalian host cells, a number of viral-based expression systems may be utilized. In cases where an adenovirus is used as an expression vector, sequences encoding TrpM8 ion channel may be ligated into an adenovirus transcription/translation complex consisting of the late promoter and tripartite leader sequence. Insertion in a non-essential E1 or E3 region of the viral genome may be used to obtain a viable virus which is capable of expressing TrpM8 ion channel in infected host cells. (Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells.

Thus, for example, the TrpM8 ion channels may be expressed in either human embryonic kidney 293 (HEK293) cells or adherent CHO cells. To maximize expression, typically all 5′ and 3′ untranslated regions (UTRs) are removed from the TrpM8 cDNA prior to insertion into a pCDN or pCDNA3 vector. The cells are transfected with individual cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual ion channels, about 24 clones are typically selected and analyzed by Northern blot analysis. TrpM8 ion channel mRNAs are generally detectable in about 50% of the G418-resistant clones analyzed.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding TrpM8 ion channel. Such signals include the ATG initiation codon and adjacent sequences. In cases where sequences encoding TrpM8 ion channel and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the connect reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of stably expressing TlpM8 ion channel can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tk⁻ or apr⁻ cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G-418 (Colbere-Garapin, F. et al (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51.) Recently, the use of visible markers has gained popularity with such markers as anthocyanins, β-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding TrpM8 ion channel is inserted within a marker gene sequence, transformed cells containing sequences encoding TrpM8 ion channel can be identified by the absence of marker gene function. Alternatively, a marker gene can be placed in tandem with a sequence encoding TrpM8 ion channel under the control of a single promoter. Expression of the marker gene in response to induction or selection usually indicates expression of the tandem gene as well.

Alternatively, host cells which contain the nucleic acid sequence encoding TrpM8 ion channel and express TrpM8 ion channel may be identified by a variety of procedures known to those of skill in the art. These procedures include, but are not limited to, DNA—DNA or DNA-RNA hybridizations and protein bioassay or immunoassay techniques which include membrane, solution, or chip based technologies for the detection and/or quantification of nucleic acid or protein sequences.

The presence of polynucleotide sequences encoding TrpM8 ion channel can be detected by DNA—DNA or DNA-RNA hybridization or amplification using probes or fragments or fragments of polynucleotides encoding TrpM8 ion channel. Nucleic acid amplification based assays involve the use of oligonucleotides or oligomers based on the sequences encoding TrpM8 ion channel to detect transformants containing DNA or RNA encoding TrpM8 ion channel.

A variety of protocols for detecting and measuring the expression of TrpM8 ion channel, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on TrpM8 ion channel is preferred, but a competitive binding assay may be employed. These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding TrpM8 ion channel include oligolabeling, nick translation, end-labeling, or PCR amplification using a labeled nucleotide. Alternatively, the sequences encoding TrpM8 ion channel, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), GE Healthcare (UK) and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding TrpM8 subunits may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode TrpM8 subunits may be designed to contain signal sequences which direct secretion of TrpM8 subunits through a prokaryotic or eukaryotic cell membrane. Other constructions may be used to join sequences encoding TrpM8 subunit to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the TrpM8 subunit encoding sequence may be used to facilitate purification. One such expression vector provides for expression of a fusion protein containing TrpM8 subunit and a nucleic acid encoding 6 histidine residues preceding a thioredoxin or an enterokinase cleavage site. The histidine residues facilitate purification on immobilized metal ion affinity chromatography (IMIAC; described in Porath, J. et al. (1992) Prot. Exp. Purif. 3: 263-281), while the enterokinase cleavage site provides a means for purifying TrpM8 subunits from the fusion protein. A discussion of vectors which contain fusion proteins is provided in Kroll, D. J. et al. (1993; DNA Cell Biol. 12:441-453).

Fragments of TrpM8 subunits may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments of TrpM8 subunits may be synthesized separately and then combined to produce the full length molecule.

Biosensors

The TrpM8 polypeptides, nucleic acids, probes, antibodies, expression vectors and ligands are useful as (and for the production of) biosensors.

According to Aizawa (1988), Anal. Chem. Symp. 17: 683, a biosensor is defined as being a unique combination of a receptor for molecular recognition, for example a selective layer with immobilized antibodies or ion channels such as a TrpM8, and a transducer for transmitting the values measured. One group of such biosensors will detect the change which is caused in the optical properties of a surface layer due to the interaction of the receptor with the surrounding medium. Among such techniques may be mentioned especially ellipso-metry and surface plasmon resonance. Biosensors incorporating TrpM8 may be used to detect the presence or level of TrpM8 ligands. The construction of such biosensors is well known in the art.

Thus, cell lines expressing TrpM8 subunits may be used as reporter systems for detection of ligands such as ATP via receptor-promoted formation of [3H]inositol phosphates or other second messengers (Watt et al., 1998, J Biol Chem May 29;273(22):14053-8). Receptor-ligand biosensors are also described in Hoffman et al., 2000, Proc Natl Acad Sci USA Oct 10;97(21):11215-20. Optical and other biosensors comprising TrpM8 may also be used to detect the level or presence of interaction with G-proteins and other proteins, as described by, for example, Figler et al, 1997, Biochemistry December 23;36(51):16288-99 and Sarrio et al., 2000, Mol Cell Biol 2000 July;20(14):5164-74). Sensor units for biosensors are described in, for example, U.S. Pat. No. 5,492,840.

Screening Assays

The TrpM8 polypeptide, including homologues, variants, and derivatives, whether natural or recombinant, may be employed in a screening process for compounds which bind the TrpM8 ion channel and which activate (agonists) or inhibit activation of (antagonists or blockers) of TrpM8. Thus, such polypeptides may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

TrpM8 ion channel polypeptides are responsible for many biological functions, including many pathologies. Accordingly, it is desirous to find compounds and drugs which stimulate TrpM8 ion channels on the one hand and which can inhibit the function of TrpM8 ion channels on the other hand. In general, agonists and antagonists are employed for therapeutic and prophylactic purposes for such conditions as anxiety, stress, depression cancer or pain.

Rational design of candidate compounds likely to be able to interact with TrpM8 ion channel proteins may be based upon structural studies of the molecular shapes of a polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the TrpM8 ion channel polypeptide on the surface thereof. Such cells include cells from animals, yeast, Drosophila or E. coli. Cells expressing TrpM8 (or cell membrane containing the expressed protein) are then contacted with a test compound to observe binding, or stimulation or inhibition of a functional response. For example, Xenopus oocytes may be injected with TrpM8 mRNA or polypeptide, and currents induced by exposure to test compounds measured by use of voltage clamps measured, as described in further detail elsewhere.

Instead of testing each candidate compound individually with the TlpM8 ion channel, a library or bank of candidate ligands may advantageously be produced and screened. Thus, for example, a bank of over 200 putative ligands has been assembled for screening. The bank comprises: transmitters, hormones and chemokines known to act via an ion channel, naturally occurring compounds which may be putative agonists for an ion channel, non-mammalian, biologically active peptides for which a mammalian counterpart has not yet been identified; and compounds not found in nature, but which activate ion channels with unknown natural ligands.

This bank may be used to screen the TrpM8 ion channel for known ligands, using both functional (i.e. calcium, microphysiometer, FLIPR assay, whole cell electrophysiology, oocyte electrophysiology, etc, see elsewhere) as well as binding assays as described in further detail elsewhere. However, a large number of mammalian receptors exist for which there remains, as yet, no cognate activating ligand (agonist) or deactivating ligand (antagonist). Thus, active ligands for these receptors may not be included within the ligands banks as identified to date. Accordingly, TrpM8 may also be functionally screened (using calcium, microphysiometer, ooyte electrophysiology, etc., functional screens) against tissue extracts to identify natural ligands. Extracts that produce positive functional responses can be sequentially subfractionated, with the fractions being assayed as described here, until an activating ligand is isolated and identified.

Ion channels which are expressed in HEK 293 cells have been shown to be coupled functionally to activation of PLC and calcium mobilization and/or cAMP stimulation or inhibition. One screening technique therefore includes the use of cells which express TrpM8 (for example, transfected Xenopus oocytes, CHO or HEK293 cells) in a system which measures extracellular pH or intracellular calcium changes caused by channel activity. In this technique, compounds may be contacted with cells expressing the TrpM8 polypeptide. A second messenger response, e.g., signal transduction, pH changes, or preferably changes in calcium level, is then measured to determine whether the potential compound activates or inhibits the channel.

Preferably, the response is a change in calcium level; such an assay is referred to for convenience as a “Functional Assay for TrpM8 (Calcium Concentration)”.

In such experiments, basal calcium levels in the HEK 293 cells in transfected or vector control cells are observed to be in the normal, 100 nM to 200 nM, range. HEK 293 cells expressing homomeric or heteromeric TrpM8 ion channels or recombinant homomeric or heteromeric TrpM8 ion channels are loaded with fura 2 and in a single day more than 150 selected ligands or tissue/cell extracts are evaluated for agonist induced calcium mobilization. Similarly, HEK 293 cells expressing TrpM8 ion channel or recombinant TrpM8 ion channel are evaluated for the increase or decrease of Ca²⁺ flux. Agonists presenting a calcium transient are tested in vector control cells to determine if the response is unique to the transfected cells expressing the ion channel.

Another method involves screening for ion channel inhibitors by determining inhibition or stimulation of TrpM8 ion channels. Such a method involves transfecting a eukaryotic cell with the TrpM8 subunits either alone to form a homomeric channel or with other Trp channel subunits to form a heteromeric channel to express the ion channel on the cell surface. The cell is then exposed to potential antagonists in the presence of the TrpM8 ion channel. The cell can be tested using whole cell electrophsysiology to determine the changes in the conductance or kinetics of the current.

Another method for detecting agonists or antagonists of TrpM8 is the yeast based technology as described in U.S. Pat. No. 5,482,835, incorporated by reference herein.

In a preferred embodiment, the screen employs detection of a change in conductance to screen for agonists and antagonists of TrpM8. Specifically, we disclose a method in which antagonists of TrpM8 lower the conductance of a suitably transfected cell. Preferably, the conductance is lowered by 10%, 20%, 30%, 40%, 50%, 60%, 70% or more in the presence of an antagonist of TrpM8. Preferably, the conductance is lowered by 1 pS, 2 pS, 3 pS, 4pS, 5pS, 10pS, 15pS, 25pS, 35pS, 45pS, 60pS, 70pS or more in the presence of an antagonist of TrpM8.

We further disclose a method in which agonists of TrpM8 increase the conductance of a suitably transfected cell. Preferably, the conductance is increased by 10%, 20%, 30%, 40%, 50%, 60%, 70% or more in the presence of an agonist of TrpM8. Preferably, the conductance is increased by 1p S, 2p S, 3p S, 4p S, 5p S, 10pS, 15p S, 25p S, 35p S, 45pS, 60pS, 70pS or more in the presence of an agonist of TrpM8.

In a further preferred embodiment, the screen employs detection of a change in intracellular calcium concentration to screen for agonists and antagonists of TrpM8. Preferably, the screen employs a function assay as set out above under “Functional Assay of TrpM8 (Calcium Concentration)”

Specifically, we disclose a method in which antagonists of TrpM8 lower the calcium concentration of a suitably transfected cell. Preferably, the calcium concentration is lowered by 10%, 20%, 30%, 40%, 50%, 60%, 70% or more in the presence of an antagonist of TrpM8. Preferably, the calcium concentration is lowered by 1p S, 2p S, 3p S, 4p S, 5p S, 10p S, 15p S, 25p S, 35p S, 45p S, 60p S, 70p S or more in the presence of an antagonist of TrpM8.

We further disclose a method in which agonists of TrpM8 increase the calcium concentration of a suitably transfected cell. Preferably, the conductance is increased by 10%, 20%, 30%, 40%, 50%, 60%, 70% or more in the presence of an agonist of TrpM8. Preferably, the calcium concentration is increased by 1p S, 2p S, 3p S, 4p S, 5p S, 10p S, 15p S, 25p S, 35p S, 45p S, 60p S, 70p S or more in the presence of an agonist of TrpM8.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a TrpM8 polypeptide or peptide are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaiyotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses. Competitive assays are particularly useful, where the cells expressing the library of compounds are contacted or incubated with a labelled antibody known to bind to a TrpM8 polypeptide, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a TrpM8 polypeptide, and is described in detail in International Patent application No. WO 84/03564 (Commonwealth Serum Labs.), published on Sep. 13 1984. First, large numbers of different small peptide test compounds are synthesized on a solid substrate, e.g., plastic pins or some other appropriate surface; see Fodor et al. (1991). Then all the pins are reacted with solubilized TrpM8 polypeptide and washed. The next step involves detecting bound polypeptide. Compounds which interact specifically with the polypeptide will thus be identified.

Ligand binding assays provide a direct method for ascertaining pharmacology and are adaptable to a high throughput format. The purified ligand may be radiolabeled to high specific activity (50-2000 Ci/mmol) for binding studies. A determination is then made that the process of radiolabeling does not diminish the activity of the ligand towards its target. Assay conditions for buffers, ions, pH and other modulators such as nucleotides are optimized to establish a workable signal to noise ratio for both membrane and whole cell receptor or ion channel sources. For these assays, specific binding is defined as total associated radioactivity minus the radioactivity measured in the presence of an excess of unlabeled competing ligand. Where possible, more than one competing ligand is used to define residual non-specific binding.

The assays may simply test binding of a candidate compound wherein adherence to the cells bearing the receptor or ion channel is detected by means of a label directly or indirectly associated with the candidate compound or in an assay involving competition with a labeled competitor. Further, these assays may test whether the candidate compound results in a signal generated by activation of the target, using detection systems appropriate to the cells bearing the target at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing a TrpM8 polypeptide to form a mixture, measuring TrpM8 ion channel activity in the mixture, and comparing the TrpM8 ion channel activity of the mixture to a standard.

The TrpM8 subunit cDNA, protein and antibodies to the protein may also be used to configure assays for detecting the effect of added compounds on the production of TrpM8 subunit mRNA and protein in cells. For example, an ELISA may be constructed for measuring secreted or cell associated levels of TrpM8 subunit protein using monoclonal and polyclonal antibodies by standard methods known in the art, and this can be used to discover agents which may inhibit or enhance the production of TrpM8 subunit (also called antagonist or agonist, respectively) from suitably manipulated cells or tissues. Standard methods for conducting screening assays are well understood in the art.

Examples of potential TrpM8 ion channel antagonists and blockers include antibodies or, in some cases, nucleotides and their analogues, including purines and purine analogues, oligonucleotides or proteins which are closely related to the ligand of the TrpM8 ion channel, e.g., a fragment of the ligand, or small molecules which bind to the ion channel but do not elicit a response, so that the activity of the channel is prevented.

We there therefore also provide a compound capable of binding specifically to a TrpM8 polypeptide and/or peptide.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Preferably the compound is an antibody.

The materials necessary for such screening to be conducted may be packaged into a screening kit. Such a screening kit is useful for identifying agonists, antagonists, ligands, receptors, substrates, enzymes, etc. for TrpM8 polypeptides or compounds which decrease or enhance the production of TrpM8 ion channel polypeptides. The screening kit comprises: (a) a TrpM8 polypeptide; (b) a recombinant cell expressing a TrpM8 polypeptide; (c) a cell membrane expressing a TrpM8 polypeptide; or (d) antibody to a TrpM8 polypeptide. The screening kit may optionally comprise instructions for use.

Transgenic Animals

We further disclose transgenic animals capable of expressing natural or recombinant TrpM8 ion channel, or a homologue, variant or derivative, at normal, elevated or reduced levels compared to the normal expression level. Preferably, such a transgenic animal is a non-human mammal, such as a pig, a sheep or a rodent. Most preferably the transgenic animal is a mouse or a rat.

We disclose transgenic animals in which all or a portion of the native TrpM8 gene is replaced by TrpM8 sequences from another organism. Preferably this organism is another species, most preferably a human. In highly preferred embodiments, we disclose a mouse which has substantially its entire TrpM8 gene replaced with a human TrpM8 gene. Such transgenic animals, as well as animals which are wild type for TrpM8, may be used for screening agonists and/or antagonists of TrpM8.

For example, such assays may involve exposing the wild type or transgenic animal, or a portion thereof, preferably a cell, tissue or organ of the transgenic animal, to a candidate substance, and assaying for a TrpM8 associated phenotype such as pain or stress. Cell-based screens employing cells derived from the relevant animal and assaying for effects on conductance or intracellular calcium concentration may also be conducted.

We further disclose transgenic animals comprising functionally disrupted TrpM8 gene, in which any one or more of the functions of TrpM8 as disclosed in this document is partially or totally abolished. Included are transgenic animals (“TrpM8 knockout”s) which do not express functional TrpM8 ion channel as a result of one or more loss of function mutations, including a deletion, of the TrpM8 gene.

Also included are partial loss-of-function mutants, e.g., an incomplete knockout, which may for example have deletions in selected portions of the TrpM8 gene. Such animals may be generated by selectively replacing or deleting relevant portions of the TrpM8 sequence, for example, functionally important protein domains.

Such complete or partial loss of function mutants are useful as models for TrpM8 related diseases, particularly pain or stress related diseases. An animal displaying partial-loss-of-function may be exposed to a candidate substance to identify substances which enhance the phenotype, that is to say, to increase (in the case of TrpM8) the hypoalgesia or reduction of stress level phenotype observed. Other parameters such as reduction in conductance or reduction in intracellular calcium levels may also be detected using the methods identified elsewhere in this document.

Partial and complete knockouts may also be used to identify selective agonists and/or antagonists of TrpM8. For example, an agonist and/or antagonist may be administered to a wild type and a TrpM8 deficient animal (knockout). A selective agonist or antagonist of TrpM8 will be seen to have an effect on the wild type animal but not in the TrpM8 deficient animal. In detail, a specific assay is designed to evaluate a potential drug (a candidate ligand or compound) to determine if it produces a physiological response in the absence of TrpM8 ion channel. This may be accomplished by administering the drug to a transgenic animal as discussed above, and then assaying the animal for a particular response. Analogous cell-based methods employing cells derived from the relevant animal and assaying for effects on conductance or intracellular calcium concentration may also be conducted. Such animals may also be used to test for efficacy of drugs identified by the screens described in this document.

In another embodiment, a transgenic animal having a partial loss-of-function phenotype is employed for screening. In such an embodiment, the screen may involve assaying for partial or complete restoration or reversion to the wild type phenotype. Cell-based screens employing cells derived from the relevant animal and assaying for effects on conductance or intracellular calcium concentration may also be conducted. A candidate compound which is found to be capable of such can be regarded as a TrpM8 agonist or analogue. Such agonists may be used for example to restore or increase sensitivity to stimuli, for example pain, or to increase stress levels in an individual.

In preferred embodiments, the transgenic TrpM8 animals, particularly TrpM8 knockouts (complete loss of function), display the phenotypes set out in the Examples, preferably as measured by the tests set out therein. Thus, the TrpM8 animals, particularly TrpM8 knockouts, preferably display any one or more of the following: decreased grip strength, propensity to drink more then wild-type mice, lower sensitivity to pain (hypoalgesia), lowered stress, decreased blood plasma corticosterone levels.

In highly preferred embodiments, the transgenic TrpM8 animals, particularly TrpM8 knockouts, display at least 10%, preferably at least 20%, more preferably at least 30%, 40%, 50%, 60%, 70%, 80%, 90%, or 100% higher or lower (as the case may be) of the measured parameter as compared to the corresponding wild-type mice. Thus, for example, TrpM8 knockouts have an increased pain threshold in response to the Tail Flick test set out in the examples, of 1 second, 2 seconds, 5 seconds, 10 seconds, 30 seconds or more, or 5%, 10%, 20%, 50% or more when compared to wild type mice. When measured in an Open Field analysis as set out in the Examples, TrpM8 deficient mice preferably have an increased permance time for central zone of 3, 4, 5, 6, 7, 8, 9, 10, 11, 12, 13, 14, 15, 20 seconds or more, or an increased distance moved in central zone of 15, 20, 25, 30, 50 cm or more, when compared to wild type mice.

It will be evident that the phenotypes now disclosed for TrpM8 deficient transgenic animals may be usefully employed in a screen using wild type animals, to detect compounds which cause similar effects to loss-of-function of TrpM8. In other words, a wild type animal may be exposed to a candidate compound, and a change in a relevant TrpM8 phenotype observed, such as hypoalgesia, reduction in sensitivity to pain, reduction in stress levels, reduction in corticosterone levels, etc, to identify modulators of TrpM8 function, particularly antagonists. Cellular phenotypes such as reduction in conductance or reduction in intracellular calcium levels may also be detected using the methods identified elsewhere in this document.

A compound identified by such a screen could be used as an antagonist of TrpM8, e.g, as an analgesic or a stress reliever, particularly for the treatment or relief of a TrpM8 associated disease.

The screens described above may involve observation of any suitable parameter, such as a behavioural, physiological or biochemical response. Preferred responses include physiological responses and may comprise one or more of the following: changes to disease resistance; altered inflammatory responses; altered tumour susceptibility: a change in blood pressure; neovascularization; a change in eating behavior; a change in body weight; a change in bone density; a change in body temperature; insulin secretion; gonadotropin secretion; nasal and bronchial secretion; vasoconstriction; loss of memory; anxiety; changed anxiety state; hyporeflexia or hyperreflexia; pain or stress responses.

Biochemical parameters may also be employed, such as a change in conductance or intracellular calcium concentration. Preferably, the conductance is measured using the “Functional Assay for TrpM8 (Conductance)” and the intracellular calcium concentration is measured using the “Functional Assay for TrpM8 (Calcium Concentration”. This is particularly useful in cell-based screens.

In preferred embodiments, the conductance of a cell (for example a wild type or partial loss-of-function cell) exposed to a TrpM8 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%. In preferred embodiments, this is measured using the “Functional Assay for TrpM8 (Conductance)” described elsewhere in this document.

In preferred embodiments, the intracellular calcium concentration of a cell (for example a wild type or partial loss-of-function cell) exposed to a TrpM8 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%. In preferred embodiments, this is measured using the “Functional Assay for TrpM8 (Intracellular Calcium Concentration)” described elsewhere in this document.

In preferred embodiments, the corticosterone levels of a wild type or TrpM8 partial knockout animal exposed to a TrpM8 agonist is increased by at least 10%, preferably at least 20%, more preferably at least +30%, more preferably at least 40%, more preferably at least 50%, more preferably at least 60%, more preferably at least 70%, more preferably at least 80%, more preferably at least 90%.

In preferred embodiments, antagonists of TrpM8 are such that wild type or partial loss-of-function animals exposed to such antagonists exhibit at least partial identity of phenotype, to at least a partial degree, as TrpM8 partial or complete loss-of-function mutants. That is to say, preferred antagonists are those which cause hypoalgesia, or reduction of stress, or reduction in serum corticosterone levels, or reduction in conductance, or reduction in intracellular calcium levels, or any combination of the above. Preferably, the relevant phenotype is expressed to the same degree as a TrpM8 knock-out animal.

In preferred embodiments, the conductance of a wild type or partial loss-of-function cell exposed to a TrpM8 antagonist is within +80%, preferably within +70%, more preferably within +60%, more preferably within +50%, more preferably within +40%, more preferably within +30%, more preferably within +20%, more preferably within +10%, more preferably within +5%, of the conductance of a TrpM8 deficient cell. In preferred embodiments, this is measured using the “Functional Assay for TrpM8 (Conductance)” described elsewhere in this document.

In preferred embodiments, the intracellular calcium concentration of a wild type or TrpM8 partial loss-of-function cell exposed to a TrpM8 antagonist is within +80%, preferably within +70%, more preferably within +60%, more preferably within +50%, more preferably within +40%, more preferably within +30%, more preferably within +20%, more preferably within +10%, more preferably within +5%, of the intracellular calcium concentration of a TrpM8 deficient cell. In preferred embodiments, this is measured using the “Functional Assay for TrpM8 (Intracellular Calcium Concentration)” described elsewhere in this document.

In preferred embodiments, the corticosterone levels of a wild type or partial TrpM8 knockout animal exposed to a TrpM8 antagonist is within +80%, preferably within +70%, more preferably within +60%, more preferably within +50%, more preferably within +40%, more preferably within +30%, more preferably within +20%, more preferably within +10%, more preferably within +5%, of the coiticosterone levels of a TrpM8 deficient transgenic animal.

Tissues derived from the TrpM8 knockout animals may be used in binding assays to determine whether the potential drug (a candidate ligand or compound) binds to the TrpM8. Such assays can be conducted by obtaining a first ion channel preparation from the transgenic animal engineered to be deficient in TrpM8 ion channel production and a second ion channel preparation from a source known to bind any identified TrpM8 ligands or compounds. In general, the first and second ion channel preparations will be similar in all respects except for the source from which they are obtained. For example, if brain tissue from a transgenic animal (such as described above and below) is used in an assay, comparable brain tissue from a normal (wild type) animal is used as the source of the second ion channel preparation. Each of the ion channel preparations is incubated with a ligand known to bind to TrpM8 ion channels, both alone and in the presence of the candidate ligand or compound. Preferably, the candidate ligand or compound will be examined at several different concentrations.

The extent to which binding by the known ligand is displaced by the test compound is determined for both the first and second ion channel preparations. Tissues derived from transgenic animals may be used in assays directly or the tissues may be processed to isolate membranes or membrane proteins, which are themselves used in the assays. A preferred transgenic animal is the mouse. The ligand may be labeled using any means compatible with binding assays. This would include, without limitation, radioactive, enzymatic, fluorescent or chemiluminescent labeling (as well as other labelling techniques as described in further detail above).

Furthermore, antagonists of TrpM8 ion channel may be identified by administering candidate compounds, etc, to wild type animals expressing functional TrpM8, and animals identified which exhibit any of the phenotypic characteristics associated with reduced or abolished expression of TrpM8 function.

Detailed methods for generating non-human transgenic animal are described in further detail below. Transgenic gene constructs can be introduced into the germ line of an animal to make a transgenic mammal. For example, one or several copies of the construct may be incorporated into the genome of a mammalian embryo by standard transgenic techniques.

In an exemplary embodiment, the transgenic non-human animals are produced by introducing transgenes into the germline of the non-human animal. Embryonal target cells at various developmental stages can be used to introduce transgenes. Different methods are used depending on the stage of development of the embryonal target cell. The specific line(s) of any animal are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the TrpM8 transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity may also be conducted. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this document, a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. There will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the methods described here will include exogenous genetic material. As set out above, the exogenous genetic material will, in certain embodiments, be a DNA sequence which results in the production of a TrpM8 ion channel. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene. (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

We also provide non-human transgenic animals, where the transgenic animal is characterized by having an altered TrpM8 gene, preferably as described above, as models for TrpM8 ion channel function. Alterations to the gene include deletions or other loss of function mutations, introduction of an exogenous gene having a nucleotide sequence with targeted or random mutations, introduction of an exogenous gene from another species, or a combination thereof. The transgenic animals may be either homozygous or heterozygous for the alteration. The animals and cells derived therefrom are useful for screening biologically active agents that may modulate TrpM8 ion channel function. The screening methods are of particular use for determining the specificity and action of potential therapies for pain and cancer, particularly prostate cancer. The animals are useful as a model to investigate the role of TrpM8 ion channels in normal tissues and organs such as the brain, heart, spleen and liver and the effect on their function.

Another aspect pertains to a transgenic nonhuman animal having a functionally disrupted endogenous TrpM8 gene but which also carries in its genome, and expresses, a transgene encoding a heterologous TrpM8 protein (i.e., a TrpM8 from another species). Preferably, the animal is a mouse and the heterologous TrpM8 is a human TrpM8. An animal, or cell lines derived from such an animal, which has been reconstituted with human TrpM8, can be used to identify agents that inhibit human TrpM8 in vivo and in vitro. For example, a stimulus that induces signalling through human TrpM8 can be administered to the animal, or cell line, in the presence and absence of an agent to be tested and the response in the animal, or cell line, can be measured. An agent that inhibits human TrpM8 in vivo or in vitro can be identified based upon a decreased response in the presence of the agent compared to the response in the absence of the agent.

We also provide for a TrpM8 deficient transgenic non-human animal (a “TrpM8 subunit knock-out”). Such an animal is one which expresses lowered or no TrpM8 ion channel activity, preferably as a result of an endogenous TrpM8 ion channel genomic sequence being disrupted or deleted. Preferably, such an animal expresses no ion channel activity. More preferably, the animal expresses no activity of the TrpM8 ion channel shown as SEQ ID NO: 3 or SEQ ID NO: 5. TrpM8 ion channel knock-outs may be generated by various means known in the air, as described in further detail below.

The present disclosure also pertains to a nucleic acid construct for functionally disrupting a TrpM8 gene in a host cell. The nucleic acid construct comprises: a) a non-homologous replacement portion; b) a first homology region located upstream of the non-homologous replacement portion, the first homology region having a nucleotide sequence with substantial identity to a first TrpM8 gene sequence; and c) a second homology region located downstream of the non-homologous replacement portion, the second homology region having a nucleotide sequence with substantial identity to a second TrpM8 gene sequence, the second TrpM8 gene sequence having a location downstream of the first TrpM8 gene sequence in a naturally occurring endogenous TrpM8 gene. Additionally, the first and second homology regions are of sufficient length for homologous recombination between the nucleic acid construct and an endogenous TrpM8 gene in a host cell when the nucleic acid molecule is introduced into the host cell. In a preferred embodiment, the non-homologous replacement portion comprises an expression reporter, preferably including lacZ and a positive selection expression cassette, preferably including a neomycin phosphotransferase gene operatively linked to a regulatory element(s).

Preferably, the first and second TrpM8 gene sequences are derived from SEQ ID NO: 1, SEQ ID NO: 2 or SEQ ID NO: 4, or a homologue, variant or derivative thereof

Another aspect pertains to recombinant vectors into which the nucleic acid construct described above has been incorporated. Yet another aspect pertains to host cells into which the nucleic acid construct has been introduced to thereby allow homologous recombination between the nucleic acid construct and an endogenous TrpM8 gene of the host cell, resulting in functional disruption of the endogenous TrpM8 gene. The host cell can be a mammalian cell that normally expresses TrpM8 from the liver, brain, spleen or heart, or a pluripotent cell, such as a mouse embryonic stem cell. Further development of an embryonic stem cell into which the nucleic acid construct has been introduced and homologously recombined with the endogenous TrpM8 gene produces a transgenic nonhuman animal having cells that are descendant from the embryonic stem cell and thus carry the TrpM8 gene disruption in their genome. Animals that carry the TrpM8 gene disruption in their germline can then be selected and bred to produce animals having the TrpM8 gene disruption in all somatic and germ cells. Such mice can then be bred to homozygosity for the TrpM8 gene disruption.

Antibodies

For the purposes of this document, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. The antibodies and fragments thereof may be humanised antibodies, for example as described in EP-A-239400. Furthermore, antibodies with fully human variable regions (or their fragments), for example, as described in U.S. Pat. Nos. 5,545,807 and 6,075,181 may also be used. Neutralizing antibodies, i.e., those which inhibit biological activity of the substance amino acid sequences, are especially preferred for diagnostics and therapeutics.

Antibodies may be produced by standard techniques, such as by immunisation or by using a phage display library.

A polypeptide or peptide of may be used to develop an antibody by known techniques. Such an antibody may be capable of binding specifically to the TrpM8 ion channel protein or homologue, fragment, etc.

If polyclonal antibodies are desired, a selected mammal (e.g., mouse, rabbit, goat, horse, etc.) may be immunised with an immunogenic composition comprising a relevant polypeptide or peptide. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance amino acid sequence is administered to immunologically compromised individuals for the purpose of stimulating systemic defence.

Serum from the immunised animal is collected and treated according to known procedures. If serum containing polyclonal antibodies to an epitope obtainable from a TrpM8 polypeptide contains antibodies to other antigens, the polyclonal antibodies can be purified by immunoaffinity chromatography. Techniques for producing and processing polyclonal antisera are known in the art. In order that such antibodies may be made, we also provide amino acid sequences of TrpM8 or fragments thereof haptenised to another amino acid sequence for use as immunogens in animals or humans.

Monoclonal antibodies directed against epitopes obtainable from a TrpM8 polypeptide or peptide can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the trioma technique, the human B-cell hybridoma technique (Kosbor et al (1983) Immunol Today 4:72; Cote et al (1983) Proc Natl Acad Sci 80:2026-2030) and the EBV-hybridoma technique (Cole et al., Monoclonal Antibodies and Cancer Therapy, pp. 77-96, Alan R. Liss, Inc., 1985).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from a TrpM8 polypeptide or peptide are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the polypeptide or peptide may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse WD et al (1989) Science 256:1275-1281).

Techniques for the production of single chain antibodies (U.S. Pat. No. 4,946,778) can also be adapted to produce single chain antibodies to TrpM8 polypeptides. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against TrpM8 ion channel polypeptides may also be employed to treat pain and cancer, particularly neuropathic pain and prostate cancer.

Diagnostic Assays

This disclosure also relates to the use of TrpM8 ion channel polynucleotides and polypeptides (as well as homologues, variants and derivatives thereof) for use in diagnosis as diagnostic reagents or in genetic analysis. Nucleic acids complementary to or capable of hybridising to TrpM8 ion channel nucleic acids (including homologues, variants and derivatives), as well as antibodies against TrpM8 polypeptides are also useful in such assays.

Detection of a mutated form of the TrpM8 ion channel gene associated with a dysfunction will provide a diagnostic tool that can add to or define a diagnosis of a disease or susceptibility to a disease which results from under-expression, over-expression or altered expression of TrpM8 ion channel. Individuals carrying mutations in the TrpM8 ion channel gene (including control sequences) may be detected at the DNA level by a variety of techniques.

For example, DNA may be isolated from a patient and the DNA polymoiphism pattern of TrpM8 determined. The identified pattern is compared to controls of patients known to be suffering from a disease associated with over; under-, or abnormal expression of TrpM8. Patients expressing a genetic polymorphism pattern associated with TrpM8 associated disease may then be identified. Genetic analysis of the TrpM8 ion channel gene may be conducted by any technique known in the art. For example, individuals may be screened by determining DNA sequence of a TrpM8 allele, by RFLP or SNP analysis, etc. Patients may be identified as having a genetic predisposition for a disease associated with the over-, under-, or abnormal expression of TrpM8 by detecting the presence of a DNA polymorphism in the gene sequence for TrpM8 or any sequence controlling its expression.

Patients so identified can then be treated to prevent the occurrence of TrpM8 associated disease, or more aggressively in the early stages of TrpM8 associated disease to prevent the further occurrence or development of the disease. TrpM8 associated diseases include stress, anxiety, depression, pain and cancer, particularly neuropathic pain and prostate cancer.

We further disclose a kit for the identification of a patient's genetic polymorphism pattern associated with TrpM8 associated disease. The kit includes DNA sample collecting means and means for determining a genetic polymorphism pattern, which is then compared to control samples to determine a patient's susceptibility to TrpM8 associated disease. Kits for diagnosis of a TrpM8 associated disease comprising TrpM8 polypeptide and/or an antibody against such a polypeptide (or fragment of it) are also provided.

Nucleic acids for diagnosis may be obtained firm a subject's cells, such as from blood, urine, saliva, tissue biopsy or autopsy material. In a preferred embodiment, the DNA is obtained from blood cells obtained from a finger prick of the patient with the blood collected on absorbent paper. In a further preferred embodiment, the blood will be collected on an AmpliCard.™. (University of Sheffield, Department of Medicine and Pharmacology, Royal Hallamshire Hospital, Sheffield, England S10 2JF).

The DNA may be used directly for detection or may be amplified enzymatically by using PCR or other amplification techniques prior to analysis. Oligonucleotide DNA primers that target the specific polymorphic DNA region within the genes of interest may be prepared so that in the PCR reaction amplification of the target sequences is achieved. RNA or cDNA may also be used as templates in similar fashion. The amplified DNA sequences from the template DNA may then be analyzed using restriction enzymes to determine the genetic polymorphisms present in the amplified sequences and thereby provide a genetic polymorphism profile of the patient. Restriction fragments lengths may be identified by gel analysis. Alternatively, or in conjunction, techniques such as SNP (single nucleotide polymorphisms) analysis may be employed.

Deletions and insertions can be detected by a change in size of the amplified product in comparison to the normal genotype. Point mutations can be identified by hybridizing amplified DNA to labeled TrpM8 ion channel nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, eg., Myers et al, Science (1985)230:1242. Sequence changes at specific locations may also be revealed by nuclease protection assays, such as RNAse and S1 protection or the chemical cleavage method. See Cotton et al., Proc Natl Acad Sci USA (1985) 85: 4397-4401. In another embodiment, an array of oligonucleotides probes comprising the TrpM8 ion channel nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

Single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (992) Genet Anal Tech Appl 9:73-79). Single-stranded DNA fragments of sample and control TrpM8 nucleic acids may be denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

The diagnostic assays offer a process for diagnosing or determining a susceptibility to infections such as pain and cancer, particularly neuropathic pain and prostate cancer through detection of mutation in the TrpM8 gene by the methods described.

The presence of TrpM8 polypeptides and nucleic acids may be detected in a sample. Thus, infections and diseases as listed above can be diagnosed by methods comprising determining from a sample derived from a subject an abnormally decreased or increased level of the TrpM8 polypeptide or TrpM8 ion channel mRNA. The sample may comprise a cell or tissue sample from an organism suffering or suspected to be suffering from a disease associated with increased, reduced or otherwise abnormal TrpM8 expression, including spatial or temporal changes in level or pattern of expression. The level or pattern of expression of TrpM8 in an organism suffering from or suspected to be suffering from such a disease may be usefully compared with the level or pattern of expression in a normal organism as a means of diagnosis of disease.

In general therefore, we disclose a method of detecting the presence of a nucleic acid comprising a TrpM8 nucleic acid in a sample, by contacting the sample with at least one nucleic acid probe which is specific for said nucleic acid and monitoring said sample for the presence of the nucleic acid. For example, the nucleic acid probe may specifically bind to the TrpM8 subunit nucleic acid, or a portion of it, and binding between the two detected, the presence of the complex itself may also be detected. Furthermore, we encompass a method of detecting the presence of a TrpM8 polypeptide by contacting a cell sample with an antibody capable of binding the polypeptide and monitoring said sample for the presence of the polypeptide. This may conveniently be achieved by monitoring the presence of a complex formed between the antibody and the polypeptide, or monitoring the binding between the polypeptide and the antibody. Methods of detecting binding between two entities are known in the art, and include FRET (fluorescence resonance energy transfer), surface plasmon resonance, etc.

Decreased or increased expression can be measured at the RNA level using any of the methods well known in the art for the quantitation of polynucleotides, such as, for example, PCR, RT-PCR, RNAse protection, Northern blotting and other hybridization methods. Assay techniques that can be used to determine levels of a protein, such as TrpM8, in a sample derived from a host are well-known to those of skill in the art. Such assay methods include radioimmunoassay, competitive-binding assays, Western Blot analysis and ELISA assays.

We further disclose a diagnostic kit for a disease or susceptibility to a disease (including an infection), for example, pain and cancer, particularly neuropathic pain and prostate cancer. The diagnostic kit comprises a TrpM8 polynucleotide or a fragment thereof; a complementary nucleotide sequence; a TrpM8 polypeptide or a fragment thereof, or an antibody to a TrpM8 polypeptide.

Chromosome Assays

The nucleotide sequences described here are also valuable for chromosome identification. The sequence is specifically targeted to and can hybridize with a particular location on an individual human chromosome. As described above, human TrpM8 ion channel is found to map to Homo sapiens chromosome 2q37.

The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian heritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

Prophylactic and Therapeutic Methods

We further provide methods of treating an abnormal conditions related to both an excess of and insufficient amounts of TrpM8 ion channel activity.

If the activity of TrpM8 ion channel is in excess, several approaches are available. One approach comprises administering to a subject an inhibitor compound (antagonist) as hereinabove described along with a pharmaceutically acceptable carrier in an amount effective to inhibit activation by blocking binding of ligands to the TrpM8 ion channel, or by inhibiting a second signal, and thereby alleviating the abnormal condition.

In another approach, soluble forms of TrpM8 polypeptides still capable of binding the ligand in competition with endogenous TrpM8 ion channel may be administered. Typical embodiments of such competitors comprise fragments of the TrpM8 polypeptide.

In still another approach, expression of the gene encoding endogenous TrpM8 ion channels can be inhibited using expression blocking techniques. Known such techniques involve the use of antisense sequences, either internally generated or separately administered. See, for example, O'Connor, J Neurochem (1991) 56:560 in Oligodeoxvnucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of TrpM8 ion channel and its activity, several approaches are also available. One approach comprises administering to a subject a therapeutically effective amount of a compound which activates TrpM8 ion channel, i.e., an agonist as described above, in combination with a pharmaceutically acceptable carrier, to thereby alleviate the abnormal condition. Alternatively, gene therapy may be employed to effect the endogenous production of TrpM8 ion channel by the relevant cells in the subject. For example, a TrpM8 polynucleotide may be engineered for expression in a replication defective retroviral vector, as discussed above. The retroviral expression construct may then be isolated and introduced into a packaging cell transduced with a retroviral plasmid vector containing RNA encoding a TrpM8 polypeptide such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

Formulation and Administration

Peptides, such as the soluble form of TrpM8 ion channel polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The disclosure further relates to pharmaceutical packs and kits comprising one or more containers filled with one or more of the ingredients of the aforementioned compositions.

TrpM8 polypeptides and other compounds may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localize, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Pharmaceutical Compositions

We further disclose a pharmaceutical composition comprising administering a therapeutically effective amount of the TrpM8 polypeptide, polynucleotide, peptide, vector or antibody thereof and optionally a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition described here may be formulated to be delivered using a a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Vaccines

Another embodiment relates to a method for inducing an immunological response in a mammal which comprises inoculating the mammal with the TrpM8 ion channel polypeptide, or a fragment thereof, adequate to produce antibody and/or T cell immune response to protect said animal from anxiety, stress, depression, pain and cancer, particularly prostate cancer, among others.

Yet another embodiment relates to a method of inducing immunological response in a mammal which comprises delivering a TrpM8 polypeptide via a vector directing expression of TrpM8 polynucleotide in vivo in order to induce such an immunological response to produce antibody to protect said animal from diseases.

A further embodiment relates to an immunological/vaccine formulation (composition) which, when introduced into a mammalian host, induces an immunological response in that mammal to a TrpM8 polypeptide wherein the composition comprises a TrpM8 polypeptide or TrpM8 gene. The vaccine formulation may further comprise a suitable carrier.

Since the TrpM8 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Vaccines may be prepared from one or more TrpM8 polypeptides or peptides.

The preparation of vaccines which contain an immunogenic polypeptide(s) or peptide(s) as active ingredient(s), is known to one skilled in the art. Typically, such vaccines are prepared as injectables, either as liquid solutions or suspensions, solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further examples of adjuvants and other agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

Typically, adjuvants such as Amphigen (oil-in-water), Alhydrogel (aluminum hydroxide), or a mixture of Amphigen and Alhydrogel are used. Only aluminum hydroxide is approved for human use.

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The TrpM8 polypeptides may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The pharmaceutical and vaccine compositions described here may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

The term “co-administered” means that the site and time of administration of each of for example, the TrpM8 polypeptide and an additional entity such as adjuvant are such that the necessary modulation of the immune system is achieved. Thus, whilst the polypeptide and the adjuvant may be administered at the same moment in time and at the same site, there may be advantages in administering the polypeptide at a different time and to a different site from the adjuvant. The polypeptide and adjuvant may even be delivered in the same delivery vehicle—and the polypeptide and the antigen may be coupled and/or uncoupled and/or genetically coupled and/or uncoupled.

The TrpM8 polypeptide, polynucleotide, peptide, nucleotide, antibody thereof and optionally an adjuvant may be administered separately or co-administered to the host subject as a single dose or in multiple doses.

The vaccine composition and pharmaceutical compositions may be administered by a number of different routes such as injection (which includes parenteral, subcutaneous and intramuscular injection) intranasal, mucosal, oral, intra-vaginal, urethral or ocular administration.

The vaccines and pharmaceutical compositions of described here may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, may be 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Further Aspects

Further aspects and embodiments of the invention are now set out in the following numbered Paragraphs; it is to be understood that the invention encompasses these aspects:

Paragraph 1. A TrpM8 polypeptide comprising the amino acid sequence shown in SEQ ID NO. 3 or SEQ ID NO: 5, or a homologue, variant or derivative thereof.

Paragraph 2. A nucleic acid encoding a polypeptide according to Paragraph 1.

Paragraph 3. A nucleic acid according to Paragraph 2, comprising the nucleic acid sequence shown in SEQ ID NO: 1, SEQ ID NO: 2 or SEQ ID NO: 4, or a homologue, variant or derivative thereof.

Paragraph 4. A polypeptide comprising a fragment of a polypeptide according to Paragraph 1.

Paragraph 5. A polypeptide according to Paragraph 3 which comprises one or more regions which are homologous between SEQ ID NO: 3 and SEQ ID NO: 5, or which comprises one or more regions which are heterologous between SEQ ID NO: 3 and SEQ ID NO: 5.

Paragraph 6. A nucleic acid encoding a polypeptide according to Paragraph 4 or 5.

Paragraph 7. A vector comprising a nucleic acid according to Paragraph 2, 3, or 6.

Paragraph 8. A host cell comprising a nucleic acid according to Paragraph 2, 3, or 6, or vector according to Paragraph 7.

Paragraph 9. A transgenic non-human animal comprising a nucleic acid according to Paragraph 2, 3 or 6, or a vector according to Paragraph 7.

Paragraph 10. A transgenic non-human animal according to Paragraph 9 which is a mouse.

Paragraph 11. Use of a polypeptide according to Paragraph 1, 4 or 5 in a method of identifying a compound which is capable of interacting specifically with a ion channel.

Paragraph 12. Use of a transgenic non-human animal according to Paragraph 9 or 10 in a method of identifying a compound which is capable of interacting specifically with a ion channel.

Paragraph 13. A method for identifying an antagonist of TrpM8, the method comprising contacting a cell which expresses TrpM8 receptor with a candidate compound and determining whether the level of cyclic AMP (cAMP) in the cell is lowered as a result of said contacting.

Paragraph 14. A method for identifying a compound capable of lowering the endogenous level of cyclic AMP in a cell which method comprises contacting a cell which expresses TrpM8 with a candidate compound and determining whether the level of cyclic AMP (cAMP) in the cell is lowered as a result of said contacting.

Paragraph 15. A method of identifying a compound capable of binding to TrpM8 polypeptide, the method comprising contacting a TrpM8 polypeptide with a candidate compound and determining whether the candidate compound binds to the TrpM8 ion channel polypeptide.

Paragraph 16. A compound identified by a method according to any of Paragraphs 11 to 15.

Paragraph 17. A compound capable of binding specifically to a polypeptide according to Paragraph 1, 4 or 5.

Paragraph 18. Use of a polypeptide according to Paragraph 1, 4 or 5, or part thereof or a nucleic acid according to Paragraph 2, 3 or 6, in a method for producing antibodies.

Paragraph 19. An antibody capable of binding specifically to a polypeptide according to Paragraph 1, 4 or 5, or part thereof or a polypeptide encoded by a nucleotide according to Paragraph 2, 3 or 6, or part thereof.

Paragraph 20. A pharmaceutical composition comprising any one or more of the following: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19, together with a pharmaceutically acceptable carrier or diluent.

Paragraph 21. A vaccine composition comprising any one or more of the following: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19.

Paragraph 22. A diagnostic kit for a disease or susceptibility to a disease comprising any one or more of the following: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19.

Paragraph 23. A method of treating a patient suffering from a disease associated with enhanced activity of TrpM8, which method comprises administering to the patient an antagonist of TrpM8 ion channel.

Paragraph 24. A method of treating a patient suffering from a disease associated with reduced activity of TrpM8, which method comprises administering to the patient an agonist of TrpM8 ion channel.

Paragraph 25. A method according to Paragraph 23 or 24, in which the TrpM8 ion channel comprises a polypeptide having the sequence shown in SEQ ID NO: 3 or SEQ ID NO: 5.

Paragraph 26. A method for treating and/or preventing a disease in a patient, which comprises the step of administering any one or more of the following to the patient: a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; an antibody according to Paragraph 19; a pharmaceutical composition according to Paragraph 20; and a vaccine according to Paragraph 20.

Paragraph 27. An agent comprising a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and/or an antibody according to Paragraph 19, said agent for use in a method of treatment or prophylaxis of disease.

Paragraph 28. Use of a polypeptide according to Paragraph 1, 4 or 5, or part thereof; a nucleic acid according to Paragraph 2, 3 or 6, or part thereof; a vector according to Paragraph 7; a cell according to Paragraph 8; a compound according to Paragraph 16 or 17; and an antibody according to Paragraph 19, for the preparation of a pharmaceutical composition for the treatment or prophylaxis of a disease.

Paragraph 29. A non-human transgenic animal, characterised in that the transgenic animal comprises an altered TrpM8 gene.

Paragraph 30. A non-human transgenic animal according to Paragraph 29, in which the alteration is selected from the group consisting of a deletion of TlpM8, a mutation in TrpM8 resulting in loss of function, introduction of an exogenous gene having a nucleotide sequence with targeted or random mutations into TrpM8, introduction of an exogenous gene from another species into TrpM8, and a combination of any of these.

Paragraph 31. A non-human transgenic animal having a functionally disrupted endogenous TrpM8 gene, in which the transgenic animal comprises in its genome and expresses a transgene encoding a heterologous TrpM8 protein.

Paragraph 32. A nucleic acid construct for functionally disrupting a TrpM8 gene in a host cell, the nucleic acid construct comprising: (a) a non-homologous replacement portion; (b) a first homology region located upstream of the non-homologous replacement portion, the first homology region having a nucleotide sequence with substantial identity to a first TrpM8 gene sequence; and (c) a second homology region located downstream of the non-homologous replacement portion, the second homology region having a nucleotide sequence with substantial identity to a second TrpM8 gene sequence, the second TrpM8 gene sequence having a location downstream of the first TrpM8 gene sequence in a naturally occurring endogenous TrpM8 gene.

Paragraph 33. A process for producing a TrpM8 polypeptide, the method comprising culturing a host cell according to Paragraph 8 under conditions in which a nucleic acid encoding a TrpM8 polypeptide is expressed.

Paragraph 34. A method of detecting the presence of a nucleic acid according to Paragraph 2, 3 or 6 in a sample, the method comprising contacting the sample with at least one nucleic acid probe which is specific for said nucleic acid and monitoring said sample for the presence of the nucleic acid.

Paragraph 35. A method of detecting the presence of a polypeptide according to Paragraph 1, 4 or 5 in a sample, the method comprising contacting the sample with an antibody according to Paragraph 19 and monitoring said sample for the presence of the polypeptide.

Paragraph 36. A method of diagnosis of a disease or syndrome caused by or associated with increased, decreased or otherwise abnormal expression of TrpM8, the method comprising the steps of: (a) detecting the level or pattern of expression of TrpM8 in an animal suffering or suspected to be suffering from such a disease, and (b) comparing the level or pattern of expression with that of a normal animal.

Paragraph 37. A kit, method, agent or use according to any of Paragraphs 22 to 28, or a method according to Paragraph 36, in which the disease is selected from the group consisting of social anxiety, post traumatic stress disorder, phobias, social phobia, special phobias, panic disorder, obsessive compulsive disorder, acute stress, disorder, separation anxiety disorder, generalised anxiety disorder, major depression, dysthymia, bipolar disorder, seasonal affective disorder or post natal depression.

### EXAMPLES

### Example 1

### Transgenic TrpM8 Knock-Out Mouse: Construction of TrpM8 Gene Targeting Vector

The TrpM8 gene was identified bio-informatically using homology searches of genome databases. A 87 kb gapped genomic contig was assembled from various databases. This contig provided sufficient flanking sequence information to enable the design of homologous arms to clone into the targeting vector.

The murine TrpM8 gene has 23 coding exons. The targeting strategy is designed to remove part of the 15th coding exon. A 4.0 kb 5′ homologous arm and a 1.6 kb 3′ homologous arm flanking the region to be deleted are amplified by PCR and the fragments are cloned into the targeting vector. The 5′ end of each oligonucleotide primer used to amplify the arms is synthesised to contain a different recognition site for a rare-cutting restriction enzyme, compatible with the cloning sites of the vector polylinkers and absent from the arms themselves. In the case of TrpM8, the primers are designed as listed in the primer table below, with 5′ arm cloning sites of AgeI/NotI and 3′ arm cloning sites of AscI/FseI (the structure of the targeting vector used, including the relevant restriction sites, is shown in Figure X).

In addition to the arm primer pairs (5′armF/5′armR) and (3′armF/3′armR), further primers specific to the TrpM8 locus are designed for the following purposes: 5′ and 3′ probe primer pairs (5′prF/5′prR and 3′prF/3′prR) to amplify two short 150-300 bp fragments of non-repetitive genomic DNA external to and extending beyond each arm, to allow Southern analysis of the targeted locus, in isolated putative targeted clones; a mouse genotyping primer pair (hetF and hetR) which allows differentiation between wild-type, heterozygote and homozygous mice, when used in a multiplex PCR with a vector specific primer, in this case, Asc306; and lastly, a target screening primer (3′scr) which anneals upstream of the end of the 3′ arm region, and which produces a target event specific 1.7 kb amplimer when paired with a primer specific to the 3′ end of the vector (TK5IBLMNL), in this case Asc146. This amplimer can only be derived from template DNA from cells where the desired genomic alteration has occurred and allows the identification of correctly targeted cells from the background of clones containing randomly integrated copies of the vector. The location of these primers and the genomic structure of the regions of the TrpM8 locus used in the targeting strategy is shown.

TABLE 1
TrpM8 Primer Sequences

| | | |
|---|---|---|
| musTrpM8 5′prF | GGCTGTGTCCCTGTTTGCATGTACTTG | SEQ ID NO: 6 |
| musTrpM8 5′prR | GTGCTAGGGATCAAACCTAAGACCTTG | SEQ ID NO: 7 |
| musTrpM8 5′armF Age | tttaccggtGAATCTATGGATACCTGTGCTTCTGTC | SEQ ID NO: 8 |
| musTrpM8 5′armR Not | aaagcggccgcGGGAAATCTCTCCATACCATTGCTTAG | SEQ ID NO: 9 |
| musTrpM8 3′armF Asc | aaaggcgcgccGTAGGGTTTCAAGCAGGTGGTACTGAG | SEQ ID NO: 10 |
| musTrpM8 3′armR Fse | tttggccggCCCCTGAGCCTTGTACTTTGTAATCTG | SEQ ID NO: 11 |
| musTrpM8 3′scr | AGGCAGTATGTTTCCCCTTCAAATCTC | SEQ ID NO: 12 |
| musTrpM8 3′prF | TGGTAGATTTTTATGTGCAGTCTCCAG | SEQ ID NO: 13 |
| musTrpM8 3′prR | CCACCATCTTCCACAGGACTTACCTAC | SEQ ID NO: 14 |
| musTrpM8 hetF | GACACGAAGAACTGGAAGATTATCCTG | SEQ ID NO: 15 |
| musTrpM8 hetR | ACAACCTCAGTACCACCTGCTTGAAAC | SEQ ID NO: 16 |
| Asc 146 | CGCATCGCCTTCTATCGCCTTCTTGAC | SEQ ID NO: 17 |
| Asc306 | AATGGCCGCTTTTCTGGATTCATCGAC | SEQ ID NO: 18 |

The position of the homology arms is chosen to functionally disrupt the TrpM8 gene. A targeting vector is prepared where the TrpM8 region to be deleted is replaced with non-homologous sequences composed of an endogenous gene expression reporter (a frame independent lacZ gene) upstream of a selection cassette composed of a promoted neomycin phosphotransferase (neo) gene arranged in the same orientation as the TrpM8 gene.

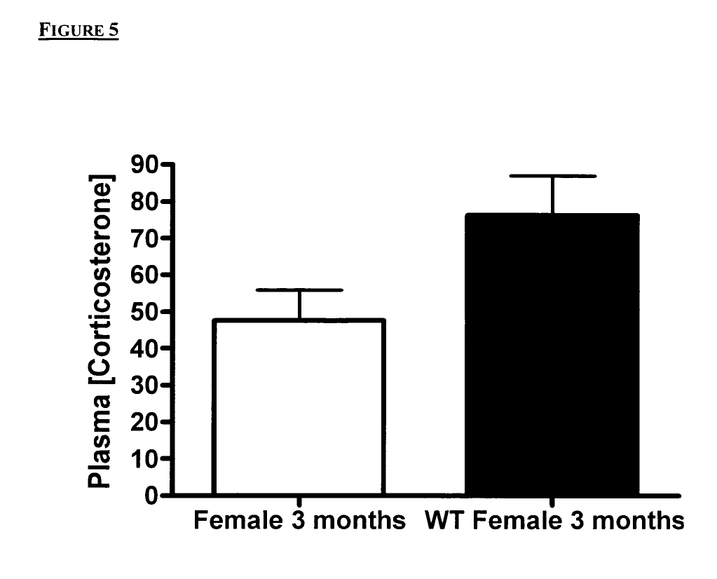

Once the 5′ and 3′ homology arms have been cloned into the targeting vector TK5IBLMNL (see FIG. 5), a large highly pure DNA preparation is made using standard molecular biology techniques. 20 μg of the freshly prepared endotoxin-free DNA is restricted with another rare-cutting restriction enzyme SwaI, present at a unique site in the vector backbone between the ampicillin resistance gene and the bacterial origin of replication. The linearized DNA is then precipitated and resuspended in 100 μl of Phosphate Buffered Saline, ready for electroporation.

Twenty-four hours following electroporation the transfected cells are cultured for 9 days in medium containing 200 μ/ml neomycin. Clones are picked into 96 well plates, replicated and expanded before being screened by PCR (using primers 3′scr and Asc146, as described above) to identify clones in which homologous recombination has occured between the endogenous TrpM8 gene and the targeting construct. Positive clones can be identified at a rate of 1 to 5%. These clones are expanded to allow replicas to be frozen and sufficient high quality DNA to be prepared for Southern blot confirmation of the targeting event using the external 5′ and 3′ probes prepared as described above, all using standard procedures (Russ et al, Nature 2000 Mar 2;404(6773):95-99). When Southern blots of DNA digested with diagnostic restriction enzymes are hybridized with an external probe, homologously targeted ES cell clones are verified by the presence of a mutant band as well an unaltered wild-type band. For instance, wild-type genomic DNA digested with BstEII will yield a band of 8.0 kb when hybridized with either external probe, while similarly digested genomic DNA containing a targeted allele will yield a ˜13 kb knockout specific band in addition.

### Example 2

### Transgenic TrpM8 Knock-Out Mouse: Generation of TrpM8 Ion Channel Deficient Mice

C57BL/6 female and male mice are mated and blastocysts are isolated at 3.5 days of gestation. 10-12 cells from a chosen clone are injected per blastocyst and 7-8 blastocysts are implanted in the uterus of a pseudopregnant F1 female. A litter of chimeric pups are born containing several high level (up to 100%) agouti males (the agouti coat colour indicates the contribution of cells descended from the targeted clone). These male chimeras are mated with female MF1 and 129 mice, and germline transmission is determined by the agouti coat colour and by PCR genotyping respectively.

PCR Genotyping is carried out on lysed tail clips, using the primers hetF and hetR with a third, vector specific primer (Asc306). This multiplex PCR allows amplification from the wild-type locus (if present) from primers hetF and hetR giving a 230 bp band. The site for hetF is deleted in the knockout mice, so this amplification will fail from a targeted allele. However, the Asc306 primer will amplify a 338 bp band from the targeted locus, in combination with the hetR primer which anneals to a region just inside the 3′ arm. Therefore, this multiplex PCR reveals the genotype of the litters as follows: wild-type samples exhibit a single 230 bp band; heterozygous DNA samples yield two bands at 230 bp and 338 bp; and the homozygous samples will show only the target specific 338 bp band.

### Example 3

### Biological Data: Gene Expression Patterns

1) RT-PCR

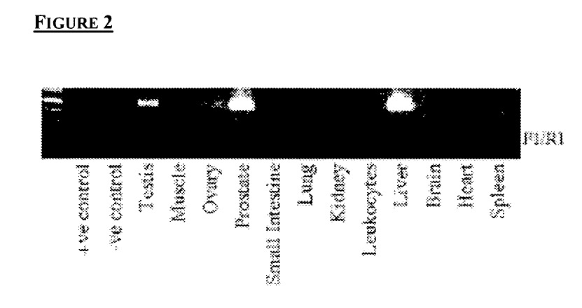

Using RT-PCR, expression of the gene is shown in the prostate, liver and testis (FIG. 2).

2) List of Lac Z stained structures

LacZ Staining

The X gal staining of dissected tissues is performed in the following manner.

Representative tissue slices are made of large organs. Whole small organs and tubes are sliced open, so fixative and stain will penetrate. Tissues are rinsed thoroughly in PBS (phosphate buffered saline) to remove blood or gut contents. Tissues are placed in fixative (PBS containing 2% formaldehyde, 0.2% glutaraldehyde, 0.02% NP40, 1mM MgCl2, Sodium deoxycholate 0.23 mM) for 30-45 minutes. Following three 5 minute washes in PBS, tissues are placed in Xgal staining solution (4 mM K Ferrocyanide, 4 mMKFerricyanide, 2 mM MgCl2, 1 mg/mlX-gal in PBS) for 18 hours at 30C. Tissues are PBS washed 3 times, postfixed for 24 hours in 4% formaldehyde, PBS washed again before storage in 70% ethanol.

Using LacZ staining, TrpM8 is found to be expressed in the DRG and in particularly in the neurons.

### Example 4

### Biological Data: Behaviour: Tail Flick Test

A tail flick analgesia test is performed using a Tail-Flick Analgesia Meter. This equipment provides an easy to use method to determine pain sensitivity accurately and reproducibly in rodents (D'Amour, F. E. and D. L. Smith, 1941, Expt. Clin. Pharmacol., 16: 179-184). The instrument has a shutter-controlled lamp as a heat source. The lamp is located below the animal to provide a less confining environment. Tail flick is detected by the automatic detection circuitry, which leaves the user's hands free to handle the animal. The animal is restrained in a ventilated tube and its tail placed on a sensing groove on top of the equipment.

Activation of an intense light beam to the tail through opening of the shutter results in discomfort at some point when the animal will flick its tail out of the beam. In the automatic mode a photo-detector detects the tail motion causing the clock to stop and the shutter to close. The total time elapsed between the shutter opening and the animal's reaction is recorded.

Responses of mutant transgenic mice are compared with age and sex matched wild-type mice. A single animal may be subjected to different heat settings to produce an increase in tail temperature no greater than 55° C.

This test is used as an indication of knockout mice response to nociceptive pain.

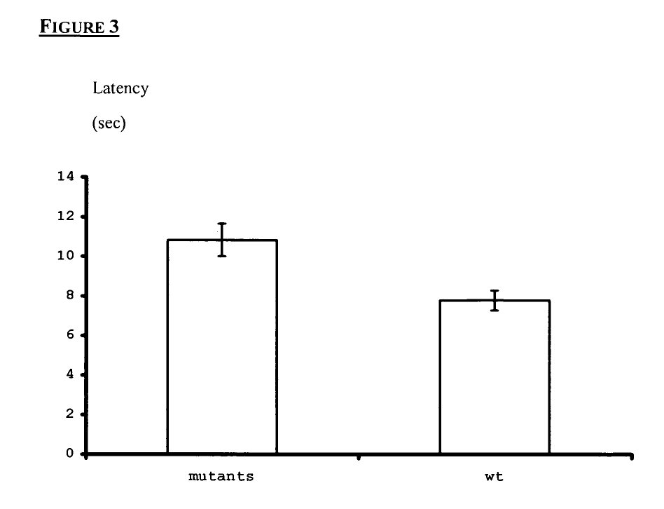

It is observed that the mutants are less sensitive to heat induced pain showing hypoalgesia. The mutants are observed to have a longer latency to flick their tails away from the heat source compared to the age and sex matched wildtype mice (FIG. 3). Accordingly, this demonstrates that TrpM8 is involved in the sensation of pain.

### Example 5

### Biological Data: Behaviour: Open Field Test

Knockout and wild-type control mice are tested in an Open Field Test. Those skilled in the art will be familiar with such test and how it is preformed. Briefly, the mice are placed in the centre of a Perspex box with clear sides and the movement of the mice over a period of time is recorded on video. The mice are analysed for distance travelled and location of the mouse at any time. Control animals usually spend most of the time moving around the periphery of the arena. Variations from this normal pattern are recorded in particular the amount of time spent in the central areas of the arena, an increase of which can mean that the animal is less anxious.

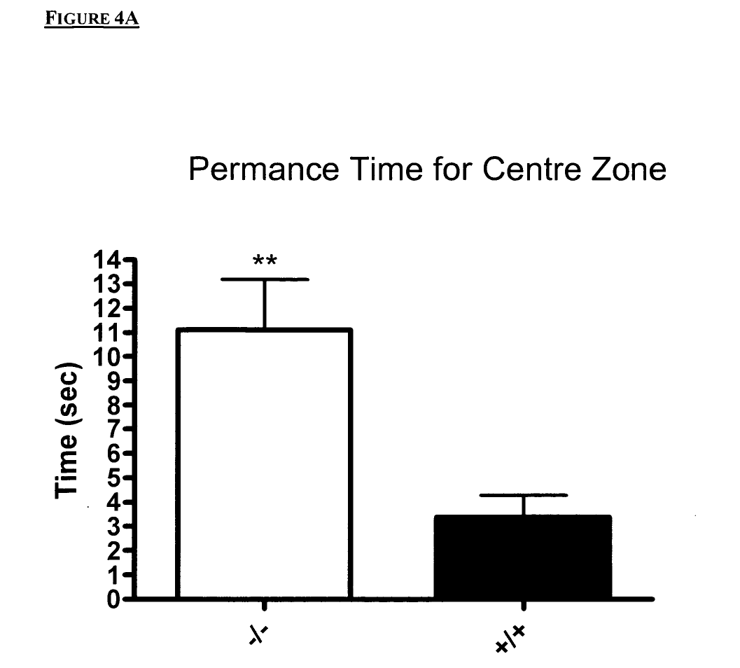

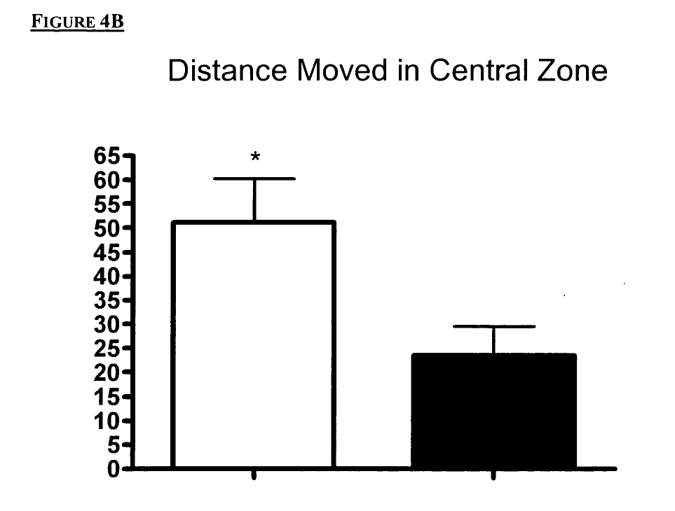

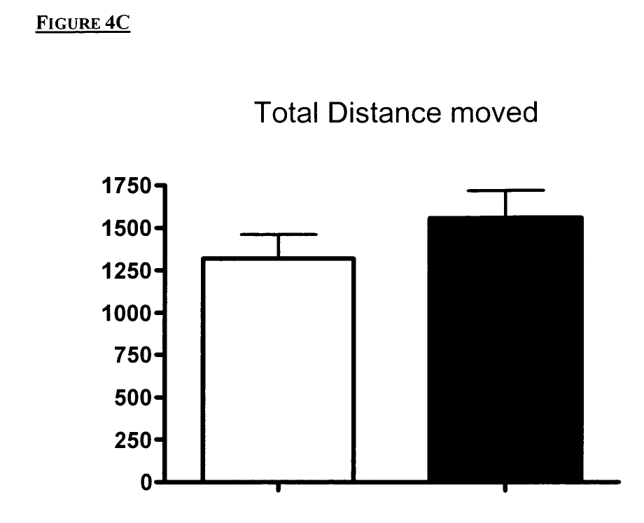

The results showed that knockout mice travelled an equal amount to the wildtype controls (FIG. 4A). However, breakdown of this data showed that the knockout mice had spent overall more time moving in the central areas of the open field arena compared to the peripheral zone (FIG. 4B). This is reflected in the distance moved in the central zone which is far greater for the knockout animals compared to the wildtypes (FIG. 4C).

### Example 6

### Biological Data: Physiology: Blood Plasma Corticosterone

Corticosterone is a hormone that is released in response to stress and anxiety. The lower the levels of corticosterone the lower the levels of anxiety and stress the animal is likely to be undergoing.

Blood plasma corticosterone are analysed from TrpM8 −/− and age matched +/+ mice using an ELISA assay. Corticosterone levels are found to be lower in the mutant animals than in the wildtypes (FIG. 5) indicating that TrpM8 mice are less anxious and stressed than their wildtype controls.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in molecular biology or related fields are intended to be within the scope of the claims. Furthermore, various combinations of the features of the following dependent claims can be made with the features of the independent claims without departing from the scope of the present invention.

### SEQUENCES

SEQ ID NO: 1

AAGAAAATCCTGCTTGACAAAAACCGTCACTTAGGAAAAGATGTCCTTTC
GGGCAGCCAGGCTCAGCATGAGGAACAGAAGGAATGACACTCTGGACAGC
ACCCGGACCCTGTACTCCAGCGCGTCTCGGAGCACAGACTTGTCTTACAG
TGAAAGCGACTTGGTGAATTTTATTCAAGCAAATTTTAAGAAACGAGAAT
GTGTCTTCTTTACCAAAGATTCCAAGGCCACGGAGAATGTGTGCAAGTGT
GGCTATGCCCAGAGCCAGCACATGGAAGGCACCCAGATCAACCAAAGTGA
GAAATGGAACTACAAGAAACACACCAAGGAATTTCCTACCGACGCCTTTG
GGGATATTCAGTTTGAGACACTGGGGAAGAAAGGGAAGTATATACGTCTG
TCCTGCGACACGGACGCGGAAATCCTTTACGAGCTGCTGACCCAGCACTG
GCACCTGAAAACACCCAACCTGGTCATTTCTGTGACCGGGGGCGCCAAGA
ACTTCGCCCTGAAGCCGCGCATGCGCAAGATCTTCAGCCGGCTCATCTAC
ATCGCGCAGTCCAAAGGTGCTTGGATTCTCACGGGAGGCACCCATTATGG
CCTGATGAAGTACATCGGGGAGGTGGTGAGAGATAACACCATCAGCAGGA
GTTCAGAGGAGAATATTGTGGCCATTGGCATAGCAGCTTGGGGCATGGTC
TCCAACCGGGACACCCTCATCAGGAATTGCGATGCTGAGGGCTATTTTTT
AGCCCAGTACCTTATGGATGACTTCACAAGAGATCCACTGTATATCCTGG
ACAACAACCACACACATTTGCTGCTCGTGGACAATGGCTGTCATGGACAT
CCCACTGTCGAAGCAAAGCTCCGGAATCAGCTAGAGAAGTATATCTCTGA
GCGCACTATTCAAGATTCCAACTATGGTGGCAAGATCCCCATTGTGTGTT
TTGCCCAAGGAGGTGGAAAAGAGACTTTGAAAGCCATCAATACCTCCATC
AAAAATAAAATTCCTTGTGTGGTGGTGGAAGGCTCGGGCCAGATCGCTGA
TGTGATCGCTAGCCTGGTGGAGGTGGAGGATGCCCTGACATCTTCTGCCG
TCAAGGAGAAGCTGGTGCGCTTTTTACCCCGCACGGTGTCCCGGCTGCCT
GAGGAGGAGACTGAGAGTTGGATCAAATGGCTCAAAGAAATTCTCGAATG
TTCTCACCTATTAACAGTTATTAAAATGGAAGAAGCTGGGGATGAAATTG
TGAGCAATGCCATCTCCTACGCTCTATACAAAGCCTTCAGCACCAGTGAG
CAAGACAAGGATAACTGGAATGGGCAGCTGAAGCTTCTGCTGGAGTGGAA
CCAGCTGGACTTAGCCAATGATGAGATTTTCACCAATGACCGCCGATGGG
AGTCTGCTGACCTTCAAGAAGTCATGTTTACGGCTCTCATAAAGGACAGA
CCCAAGTTTGTCCGCCTCTTTCTGGAGAATGGCTTGAACCTACGGAAGTT
TCTCACCCATGATGTCCTCACTGAACTCTTCTCCAACCACTTCAGCACGC
TTGTGTACCGGAATCTGCAGATCGCCAAGAATTCCTATAATGATGCCCTC
CTCACGTTTGTCTGGAAACTGGTTGCGAACTTCCGAAGAGGCTTCCGGAA
GGAAGACAGAAATGGCCGGGACGAGATGGACATAGAACTCCACGACGTGT
CTCCTATTACTCGGCACCCCCTGCAAGCTCTCTTCATCTGGGCCATTCTT
CAGAATAAGAAGGAACTCTCCAAAGTCATTTGGGAGCAGACCAGGGGCTG
CACTCTGGCAGCCCTGGGAGCCAGCAAGCTTCTGAAGACTCTGGCCAAAG
TGAAGAACGACATCAATGCTGCTGGGGAGTCCGAGGAGCTGGCTAATGAG
TACGAGACCCGGGCTGTTGAGCTGTTCACTGAGTGTTACAGCAGCGATGA
AGACTTGGCAGAACAGCTGCTGGTCTATTCCTGTGAAGCTTGGGGTGGAA
GCAACTGTCTGGAGCTGGCGGTGGAGGCCACAGACCAGCATTTCATCGCC
CAGCCTGGGGTCCAGAATTTTCTTTCTAAGCAATGGTATGGAGAGATTTC
CCGAGACACCAAGAACTGGAAGATTATCCTGTGTCTGTTTATTATACCCT
TGGTGGGCTGTGGCTTTGTATCATTTAGGAAGAAACCTGTCGACAAGCAC
AAGAAGCTGCTTTGGTACTATGTGGCGTTCTTCACCTCCCCCTTCGTGGT
CTTCTCCTGGAATGTGGTCTTCTACATCGCCTTCCTCCTGCTGTTTGCCT
ACGTGCTGCTCATGGATTTCCATTCGGTGCCACACCCCCCCGAGCTGGTC
CTGTACTCGCTGGTCTTTGTCCTCTTCTGTGATGAAGTGAGACAGTGGTA
CGTAAATGGGGTGAATTATTTTACTGACCTGTGGAATGTGATGGACACGC
TGGGGCTTTTTTACTTCATAGCAGGAATTGTATTTCGGCTCCACTCTTCT
AATAAAAGCTCTTTGTATTCTGGACGAGTCATTTTCTGTCTGGACTACAT
TATTTTCACTCTAAGATTGATCCACATTTTTACTGTAAGCAGAAACTTAG
GACCCAAGATTATAATGCTGCAGAGGATGCTGATCGATGTGTTCTTCTTC
CTGTTCCTCTTTGCGGTGTGGATGGTGGCCTTTGGCGTGGCCAGGCAAGG
GATCCTTAGGCAGAATGAGCAGCGCTGGAGGTGGATATTCCGTTCGGTCA
TCTACGAGCCCTACCTGGCCATGTTCGGCCAGGTGCCCAGTGACGTGGAT
GGTACCACGTATGACTTTGCCCACTGCACCTTCACTGGGAATGAGTCCAA
GCCACTGTGTGTGGAGCTGGATGAGCACAACCTGCCCCGGTTCCCCGAGT
GGATCACCATCCCCCTGGTGTGCATCTACATGTTATCCACCAACATCCTG
CTGGTCAACCTGCTGGTCGCCATGTTTGGCTACACGGTGGGCACCGTCCA
GGAGAACAATGACCAGGTCTGGAAGTTCCAGAGGTACTTCCTGGTGCAGG
AGTACTGCAGCCGCCTCAATATCCCCTTCCCCTTCATCGTCTTCGCTTAC
TTCTACATGGTGGTGAAGAAGTGCTTCAAGTGTTGCTGCAAGGAGAAAAA
CATGGAGTCTTCTGTCTGCTGTTTCAAAAATGAAGACAATGAGACTCTGG
CATGGGAGGGTGTCATGAAGGAAAACTACCTTGTCAAGATCAACACAAAA
GCCAACGACACCTCAGAGGAAATGAGGCATCGATTTAGACAACTGGATAC
AAAGCTTAATGATCTCAAGGGTCTTCTGAAAGAGATTGCTAATAAAATCA
AATAA

SEQ ID NO: 2

ATGTCCTTTCGGGCAGCCAGGCTCAGCATGAGGAACAGAAGGAATGACAC
TCTGGACAGCACCCGGACCCTGTACTCCAGCGCGTCTCGGAGCACAGACT
TGTCTTACAGTGAAAGCGACTTGGTGAATTTTATTCAAGCAAATTTTAAG
AAACGAGAATGTGTCTTCTTTACCAAAGATTCCAAGGCCACGGAGAATGT
GTGCAAGTGTGGCTATGCCCAGAGCCAGCACATGGAAGGCACCCAGATCA
ACCAAAGTGAGAAATGGAACTACAAGAAACACACCAAGGAATTTCCTACC
GACGCCTTTGGGGATATTCAGTTTGAGACACTGGGGAAGAAAGGGAAGTA
TATACGTCTGTCCTGCGACACGGACGCGGAAATCCTTTACGAGCTGCTGA
CCCAGCACTGGCACCTGAAAACACCCAACCTGGTCATTTCTGTGACCGGG
GGCGCCAAGAACTTCGCCCTGAAGCCGCGCATGCGCAAGATCTTCAGCCG
GCTCATCTACATCGCGCAGTCCAAAGGTGCTTGGATTCTCACGGGAGGCA
CCCATTATGGCCTGATGAAGTACATCGGGGAGGTGGTGAGAGATAACACC
ATCAGCAGGAGTTCAGAGGAGAATATTGTGGCCATTGGCATAGCAGCTTG
GGGCATGGTCTCCAACCGGGACACCCTCATCAGGAATTGCGATGCTGAGG
GCTATTTTTTAGCCCAGTACCTTATGGATGACTTCACAAGAGATCCACTG
TATATCCTGGACAACAACCACACACATTTGCTGCTCGTGGACAATGGCTG
TCATGGACATCCCACTGTCGAAGCAAAGCTCCGGAATCAGCTAGAGAAGT
ATATCTCTGAGCGCACTATTCAAGATTCCAACTATGGTGGCAAGATCCCC
ATTGTGTGTTTTGCCCAAGGAGGTGGAAAAGAGACTTTGAAAGCCATCAA
TACCTCCATCAAAAATAAAATTCCTTGTGTGGTGGTGGAAGGCTCGGGCC
AGATCGCTGATGTGATCGCTAGCCTGGTGGAGGTGGAGGATGCCCTGACA
TCTTCTGCCGTCAAGGAGAAGCTGGTGCGCTTTTTACCCCGCACGGTGTC
CCGGCTGCCTGAGGAGGAGACTGAGAGTTGGATCAAATGGCTCAAAGAAA
TTCTCGAATGTTCTCACCTATTAACAGTTATTAAAATGGAAGAAGCTGGG
GATGAAATTGTGAGCAATGCCATCTCCTACGCTCTATACAAAGCCTTCAG
CACCAGTGAGCAAGACAAGGATAACTGGAATGGGCAGCTGAAGCTTCTGC
TGGAGTGGAACCAGCTGGACTTAGCCAATGATGAGATTTTCACCAATGAC
CGCCGATGGGAGTCTGCTGACCTTCAAGAAGTCATGTTTACGGCTCTCAT
AAAGGACAGACCCAAGTTTGTCCGCCTCTTTCTGGAGAATGGCTTGAACC
TACGGAAGTTTCTCACCCATGATGTCCTCACTGAACTCTTCTCCAACCAC
TTCAGCACGCTTGTGTACCGGAATCTGCAGATCGCCAAGAATTCCTATAA
TGATGCCCTCCTCACGTTTGTCTGGAAACTGGTTGCGAACTTCCGAAGAG
GCTTCCGGAAGGAAGACAGAAATGGCCGGGACGAGATGGACATAGAACTC
CACGACGTGTCTCCTATTACTCGGCACCCCCTGCAAGCTCTCTTCATCTG
GGCCATTCTTCAGAATAAGAAGGAACTCTCCAAAGTCATTTGGGAGCAGA
CCAGGGGCTGCACTCTGGCAGCCCTGGGAGCCAGCAAGCTTCTGAAGACT
CTGGCCAAAGTGAAGAACGACATCAATGCTGCTGGGGAGTCCGAGGAGCT
GGCTAATGAGTACGAGACCCGGGCTGTTGAGCTGTTCACTGAGTGTTACA
GCAGCGATGAAGACTTGGCAGAACAGCTGCTGGTCTATTCCTGTGAAGCT
TGGGGTGGAAGCAACTGTCTGGAGCTGGCGGTGAGGCCACAGACCAGCAT
TTCATCGCCCAGCCTGGGGTCCAGATTTTCTTTCTAAGCAATGGTATGGA
GAGATTTCCCGAGACACCAAGAACTGGAAGATTATCCTGTGTCTGTTTAT
TATACCCTTGGTGGGCTGTGGCTTTGTATCATTTAGGAAGAAACCTGTCG
ACAAGCACAAGAAGCTGCTTTGGTACTATGTGGCGTTCTTCACCTCCCCC
TTCGTGGTCTTCTCCTGGAATGTGGTCTTCTACATCGCCTTCCTCCTGCT
GTTTGCCTACGTGCTGCTCATGGATTTCCATTCGGTGCCACACCCCCCCG
AGCTGGTCCTGTACTCGCTGGTCTTTGTCCTCTTCTGTGATGAAGTGAGA
CAGTGGTACGTAAATGGGGTGAATTATTTTACTGACCTGTGGAATGTGAT
GGACACGCTGGGGCTTTTTTACTTCATAGCAGGAATTGTATTTCGGCTCC
ACTCTTCTAATAAAAGCTCTTTGTATTCTGGACGAGTCATTTTCTGTCTG
GACTACATTATTTTCACTCTAAGATTGATCCACATTTTTACTGTAAGCAG
AAACTTAGGACCCAAGATTATAATGCTGCAGAGGATGCTGATCGATGTGT
TCTTCTTCCTGTTCCTCTTTGCGGTGTGGATGGTGGCCTTTGGCGTGGCC
AGGCAAGGGATCCTTAGGCAGAATGAGCAGCGCTGGAGGTGGATATTCCG
TTCGGTCATCTACGAGCCCTACCTGGCCATGTTCGGCCAGGTGCCCAGTG
ACGTGGATGGTACCACGTATGACTTTGCCCACTGCACCTTCACTGGGAAT
GAGTCCAAGCCACTGTGTGTGGAGCTGGATGAGCACAACCTGCCCCGGTT
CCCCGAGTGGATCACCATCCCCCTGGTGTGCATCTACATGTTATCCACCA
ACATCCTGCTGGTCAACCTGCTGGTCGCCATGTTTGGCTACACGGTGGGC
ACCGTCCAGGAGAACAATGACCAGGTCTGGAAGTTCCAGAGGTACTTCCT
GGTGCAGGAGTACTGCAGCCGCCTCAATATCCCCTTCCCCTTCATCGTCT
TCGCTTACTTCTACATGGTGGTGAAGAAGTGCTTCAAGTGTTGCTGCAAG
GAGAAAAACATGGAGTCTTCTGTCTGCTGTTTCAAAAATGAAGACAATGA
GACTCTGGCATGGGAGGGTGTCATGAAGGAAAACTACCTTGTCAAGATCA
ACACAAAAGCCAACGACACCTCAGAGGAAATGAGGCATCGATTTAGACAA
CTGGATACAAAGCTTAATGATCTCAAGGGTCTTCTGAAAGAGATTGCTAA
TAAAATCAAATAA

SEQ ID NO: 3

MSFRAARLSMRNRRNDTLDSTRTLYSSASRSTDLSYSESDLVNFIQANFK
KRECVFFTTKDSKATENVCKCGYAQSQHMEGTQINQSEKWNYKKHTKEFP
TDAFGDIQFETLGKKGKYIRLSCDTDAEILYELLTQHWHLKTPNLVISVT
GGAKNFALKPRNRKIFSRLIYIAQSKGAWILTGGTHYGLMKYIGEVVRDN
TISRSSEENIVAIGIAAWGMVSNRDTLIRNCDAEGYFLAQYLMDDFTRDP
LYILDNNNTHLLLVDNGCHGHPTVEAKLRNQLEKYISERTIQDSNYGGKI
PIVCFAQGGGKETLKAINTSIKNKIPCVVVEGSGQIADVIASLVEVEDAL
TSSAVKEKLVRFLPRTVSRLPEEETESWIKWLKEILECSHLLTVIKMEEA
GDEIVSNAISYALYKAFSTSEQDKDNWNGQLKLLLEWNQLDLANDEIFTN
DRRWESADLQEVMFTALIKDRPKFVRLFLENGLNLRKFLTHDVLTELFSN
HPSTLVYRNLQIAKNSYNDALLTFVWKLVANFRRGFRKEDRNGRDEMDIE
LHDVSPITRHPLQALFIWAILQNKKELSKVIWEQTRGCTLAALGASKLLK
TLAKVKNDINAAGESEELANEYETRAVELFTECYSSDEDLAEQLLVYSCE
AWGGSNCLELAVEATDQHFIAQPGVQNELSKQWYGETSRDTKNWKIILCL
FIIPLVGCGFVSFRKKPVDKHKKLLWYYVAFFTSPFVVFSWNVVFYIAFL
LLRAYVLLMDFNSVPHPPELVLYSLVFVLFCDEVRQWYVNGVNYFTDLWN
VMDTLGLFYFIAGIVFRLHSSNKSSLYSGRVIFCLDYIIFTLRLIHIFTV
SRNLGPKIIMLQRMLIDVFFFLFLFAVWMVAPGVARQGILRQNEQRWRWI
FRSVIYEPYLANFGQVPSDVDGTTYDFAHCTFTGNESKPLCVELDEHNLP
RFPEWITIPLVCIYMLSTNILLVNLLVANFGYTVGTVQENNDQVWKFQRY
FLVQEYCSRLNIPFPFIVFAYFYMVVKKCFKCCCKEKNMESSVCCFKNED
NETLAWEGVMKENYLVKINTKANDTSEEMRHRFRQLDTKLNDLKGLLKEI
ANKIK

SEQ ID NO: 4

CCTGGCTGTCCCGGAGCTTGATACATAGAAAAGACTGACCTCAGATACAC
AGAGATCCTTCTGCTTCTGTCTCCCAAGTGCTGGGATCACAGGCAAGATG
TCCTTCGAGGGAGCCAGGCTCAGCATGAGGAGCCGCAGAAATGGTACTAT
GGGCAGCACCCGGACCCTGTACTCCAGTGTATCTCGGAGCACAGACGTGT
CCTACAGTGACAGTGATTTGGTGAATTTTATTCAGGCAAATTTTAAAAAA
CGAGAATGTGTCTTCTTTACCAGAGACTCCAAGGCCATGGAGAACATATG
CAAGTGTGGTTATGCCCAGAGCCAGCACATCGAAGGCACCCAGATCAACC
AAAATGAGAAGTGGAACTACAAAAAACATACCAAGGAGTTTCCAACAGAC
GCCTTCGGGGACATTCAGTTTGAGACTCTGGGGAAGAAAGGCAAGTACTT
ACGCTTGTCCTGTGACACCGACTCTGAAACTCTCTACGAACTGCTGACCC
AGCACTGGCACCTCAAAACACCCAACCTGGTCATTTCAGTGACGGGTGGA
GCCAAAAACTTTGCTTTGAAGCCACGCATGCGCAAGATCTTCAGCAGGCT
GATTTACATCGCACAGTCTAAAGGTGCGTGGATTCTCACTGGAGGCACTC
ACTACGGCCTGATGAAGTACATAGGCGAGGTGGTGAGAGACAACACCATC
AGCAGGAACTCAGAAGAGAACATCGTGGCCATTGGCATCGCAGCATGGGG
CATGGTCTCCAACAGGGACACCCTCATCAGGAGCTGTGATGATGAGGGAC
ATTTTTCAGCTCAATACATCATGGATGACTTTACCAGAGACCCTCTATAC
ATCCTGGACAACAACCATACCCACCTGCTGCTTGTGGACAACGGTTGTCA
TGGACACCCCACAGTGGAAGCCAAGCTCCGGAATCAGCTGGAAAAGTACA
TCTCTGAGCGCACCAGTCAAGATTCCAACTATGGTGGTAAGATCCCCATC
GTGTGTTTTGCCCAAGGAGGTGGAAGAGAGACTCTAPAAGCCATCAACAC
CTCTGTCAAAAGCAAGATCCCTTGTGTGGTGGTGGAAGGCTCGGGGCAGA
TTGCTGATGTGATCGCCAGCCTGGTGGAGGTGGAGGATGTTTTAACCTCT
TCCATGGTCAAAGAGAAGCTGGTACGCTTTTTACCACGCACTGTGTCCCG
GCTGCCTGAAGAGGAAATTGAGAGCTGGATCAAATGGCTCAAAGAAATTC
TTGAGAGTTCTCACCTACTCACAGTAATTAAGATGGAAGAGGCTGGAGAT
GAGATTGTGAGCAACGCCATTTCCTATGCGCTGTACAAAGCCTTCAGCAC
TAATGAGCAAGACAAGGACAACTGGAATGGACAGCTGAAGCTTCTGCTGG
AGTGGAACCAGTTGGACCTTGCCAGTGATGAGATCTTCACCAATGATCGC
CGCTGGGAGTCTGCCGACCTTCAGGAGGTCATGTTCACGGCTCTCATAAA
GGACAGACCCAAGTTTGTCCGCCTCTTTCTGGAGAATGGCCTGAATCTGC
AGAAGTTTCTCACCAATGAAGTCCTCACAGAGCTCTTCTCCACCCACTTC
AGCACCCTAGTGTACCGGAATCTGCAGATCGCCAAGAACTCCTACAATGA
CGCACTCCTCACCTTTGTCTGGAAGTTGGTGGCAAACTTCCGTCGAAGCT
TCTGGAAAGAGGACAGAAGCAGCAGGGAGGACTTGGATGTGGAACTCCAT
GATGCATCTCTCACCACCCGGCACCCGCTGCAAGCTCTCTTCATCTGGGC
CATTCTTCAGAACAAGAAGGAACTCTCCAAGGTCATTTGGGAGCAGACCA
AAGGCTGTACTCTGGCAGCCTTGGGGGCCAGCAAGCTTCTGAAGACCCTG
GCCAAAGTTAAGAATGATATCAACGCTGCTGGGGAATCGGAGGAACTGGC
CAATGAATATGAGACCCGAGCAGTGGAGTTGTTCACCGAGTGTTACAGCA
ATGATGAAGACTTGGCAGAACAGCTACTGGTCTACTCCTGCGAAGCCTGG
GGTGGGAGCAACTGTCTGGAGCTGGCAGTGGAGGCTACAGATCAGCATTT
CATCGCTCAGCCTGGGGTCCAGAATTTCCTTTCTAAGCAATGGTATGGAG
AGATTTCCCGAGACACGAAGAACTGGAAGATTATCCTGTGTCTATTCATC
ATCCCCTTAGTGGGCTGTGGCCTCGTATCATTTAGGAGAAACCCATTGAC
AAGCACAAGAAGCTGCTGTGGTACTATGTGGCCTTCTTCACGTCGCCCTT
CGTGGTCTTCTCCTGGAACGTGGTCTTCTACATCGCCTTCCTCCTGCTGT
TTGCCTATGTGCTGCTCATGGACTTCCACTCAGTGCCACACACCCCCGAG
CTGATCCTCTACGCCCTGGTCTTCGTCCTCTTCTGTGATGAAGTGAGGCA
GTGGTACATGAACGGAGTGAATTATTTCACCGACCTATGGAACGTTATGG
ACACCCTGGGACTCTTCTACTTCATAGCGGGTATTGTATTCCGGCTCCAC
TCTTCTAATAAAAGCTCGTTGTACTCTGGGCGCGTCATTTTCTGTCTGGA
TTACATTATATTCACGCTAGGCTCATCCACATTTTCACCGTCAGCAGGAA
CTTGGGACCCAAGATTATAATGCTGCAGCGGATGCTGATCGACGTTTTCT
TCTTCCTGTTCCTCTTTGCTGTGTGGATGGTGGCCTTTGGCGTGGCCAGA
CAGGGGATCCTAAGGCAAAATGAACAGCGCTGGAGATGGATCTTCCGCTC
TGTCATCTATGAGCCCTACCTGGCCATGTTTGGCCAGGTTCCCAGTGACG
TGGATAGTACCACATATGACTTCTCCCACTGTACCTTCTCGGGAAATGAG
TCCAAGCCACTGTGTGTGGAGCTGGATGAGCACAACCTGCCCCGCTTCCC
TGAGTGGATCACCATTCCGCTGGTGTGCATCTACATGCTCTCCACCAATA
TCCTTCTGGTCAACCTCCTGGTCGCCATGTTTGGCTACACGGTAGGCATT
GTACAGGAGAACAACGACCAGGTCTGGAAATTCCAGCGGTACTTCCTGGT
GCAGGAGTACTGCAACCGCCTAAACATCCCCTTCCCCTTCGTTGTCTTCG
CTTATTTCTACATGGTGGTGAAGAAGTGTTTCAAATGCTGCTGTAAAGAG
AAGAATATGGAGTCTAATGCCTGCTGTTTCAGAAATGAGGACAATGAGAC
TTTGGCGTGGGAGGGTGTCATGAAGGAGTATTACCTTGTCAAGATCAACA
CGAAAGCCAACGACAACTCAGAGGAGATGAGGCATCGGTTTAGACAACTG
GACTCAAAGCTTAACGACCTCAAAAGTCTTCTGAAAGAGATTGCTAATAA
CATCAAGTAACCTGGCTGTCCCGGAGCTTGATACATAGAAAAGACTGACC
TCAGATACACAGAGATCCTTCTGCTTCTGTCTCCCAAGTGCTGGGATCAC
AGGCAAGATGTCCTTCGAGGGAGCCAGGCTCAGCATGAGGAGCCGCAGAA
ATGGTACTATGGGCAGCACCCGGACCCTGTACTCCAGTGTATCTCGGAGC
ACAGACGTGTCCTACAGTGACAGTGATTTGGTGAATTTTATTCAGGCAAA
TTTTAAAAAACGAGAATGTGTCTTCTTTACCAGAGACTCCAAGGCCATGG
AGAACATATGCAAGTGTGGTTATGCCCAGAGCCAGCACATCGAAGGCACC
CAGATCAACCAAAATGAGAAGTGGAACTACAAAAAACATACCAAGGAGTT
TCCAACAGACGCCTTCGGGGACATTCAGTTTGAGACTCTGGGGAAGAAAG
GCAAGTACTTACGCTTGTCCTGTGACACCGACTCTGAAACTCTCTACGAA
CTGCTGACCCAGCACTGGCACCTCAAAACACCCAACCTGGTCATTTCAGT
GACGGGTGGAGCCAACAAACTTTGCTTTGATGGCCACGCATGCGCAAGAT
CTTCAGCAGGCTGATTTACATCGCACAGTCTAAAGGTGCGTGGATTCTCA
CTGGAGGCACTCACTACGGCCTGATGAAGTACATAGGCGAGGTGGTGAGA
GACAACACCATCAGCAGGAACTCAGAAGAGAACATCGTGGCCATTGGCAT
CGCAGCATGGGGCATGGTCTCCAACAGGGACACCCTCATCAGGAGCTGTG
ATGATGAGGGACATTTTTCAGCTCAATACATCATGGATGACTTTACCAGA
GACCCTCTATACATCCTGGACAACAACCATACCCACCTGCTGCTTGTGGA
CAACGGTTGTCATGGACACCCCACAGTGGAAGCCAAGCTCCGGAATCAGC
TGGAAAAGTACATCTCTGAGCGCACCAGTCAAGATTCCAACTATGGTGGT
AAGATCCCCATCGTGTGTTTTGCCCAAGGAGGTGGAAGAGAGACTCTAAA
AGCCATCAACACCTCTGTCAAAAGCAAGATCCCTTGTGTGGTGGTGGAAG
GCTCGGGGCAGATTGCTGATGTGATCGCCAGCCTGGTGGAGGTGGAGGAT
GTTTTAACCTCTTCCATGGTCAAAGAGAAGCTGGTACGCTTTTTACCACG
CACTGTGTCCCGGCTGCCTGAAGAGGAAATTGAGAGCTGGATCAAATGGC
TCAAAGAAATTCTTGAGAGTTCTCACCTACTCACAGTAATTAAGATGGAA
GAGGCTGGAGATGAGATTGTGAGCAACGCCATTTCCTATGCGCTGTACAA
AGCCTTCAGCACTAATGAGCAAGACAAGGACAACTGGAATGGACAGCTGA
AGCTTCTGCTGGAGTGGAACCAGTTGGACCTTGCCAGTGATGAGATCTTC
ACCAATGATCGCCGCTGGGAGTCTGCCGACCTTCAGGAGGTCATGTTCAC
GGCTCTCATAAAGGACAGACCCAAGTTTGTCCGCCTCTTTCTGGAGAATG
GCCTGAATCTGCAGAAGTTTCTCACCAATGAAGTCCTCACAGAGCTCTTC
TCCACCCACTTCAGCACCCTAGTGTACCGGAATCTGCAGATCGCCAAGAA
CTCCTACAATGACGCACTCCTCACCTTTGTCTGGAAGTTGGTGGCAAACT
TCCGTCGAAGCTTCTGGAAAGAGGACAGAAGCAGCAGGGAGGACTTGGAT
GTGGAACTCCATGATGCATCTCTCACCACCCGGCACCCGCTGCAAGCTCT
CTTCATCTGGGCCATTCTTCAGAACAAGAAGGAACTCTCCAAGGTCATTT
GGGAGCAGACCAAAGGCTGTACTCTGGCAGCCTTGGGGGCCAGCAAGCTT
CTGAAGACCCTGGCCAAAGTTAAGAATGATATCAACGCTGCTGGGGAATC
GGAGGAACTGGCCAATGAATATGAGACCCGAGCAGTGGAGTTGTTCACCG
AGTGTTACAGCAATGATGAGACTTGGCAGAACAGCTACTGGTCTACTCCT
GCGAAGCCTGGGGTGGGAGCAACTGTCTGGAGCTGGCAGTGGAGGCTACA
GATCAGCATTTCATCGCTCAGCCTGGGTCCAGAATTTCCTTTCTAAGCAA
TGGTATGGAGAGATTTCCCGAGACACGAAGAACTGGAAGATTATCCTGTG
TCTATTCATCATCCCCTTAGTGGGCTGTGGCCTCGTATCATTTAGGAAGA
AACCCATTGACAAGCACAAGAAGCTGCTGTGGTACTATGTGGCCTTCTTC
ACGTCGCCCTTCGTGGTCTTCTCCTGGAACGTGGTCTTCTACATCGCCTT
CCTCCTGCTGTTTGCCTATGTGCTGCTCATGGACTTCCACTCAGTGCCAC
ACACCCCCGAGCTGATCCTCTACGCCCTGGTCTTCGTCCTCTTCTGTGAT
AAGTGAGGCAGTGGTACATGAACGGAGTGAATTATTTCACCGACCTATGG
AACGTTATGGACACCCTGGGACTCTTCTACTTCATAGCGGGTATTGTATT
CCGGCTCCACTCTTCTAATAAAAGCTCGTTGTACTCTGGGCGCGTCATTT
TCTGTCTGGATTACATTATATTCACGCTAAGGCTCATCCACATTTTCACC
GTCAGCAGGAACTTGGGACCCAAGATTATAATGCTGCAGCGGATGCTGAT
CGACGTTTTCTTCTTCCTGTTCCTCTTTGCTGTGTGGATGGTGGCCTTTG
GCGTGGCCAGACAGGGGATCCTAAGGCAAAATGAACAGCGCTGGAGATGG
ATCTTCCGCTCTGTCATCTATGAGCCCTACCTGGCCATGTTTGGCCAGGT
TCCCAGTGACGTGGATAGTACCACATATGACTTCTCCCACTGTACCTTCT
CGGGAAATGAGTCCAAGCCACTGTGTGTGGAGCTGGATGAGCACAACCTG
CCCCGCTTCCCTGAGTGGATCACCATTCCGCTGGTGTGCATCTACATGCT
CTCCACCAATATCCTTCTGGTCAACCTCCTGGTCGCCATGTTTGGCTACA
CGGTAGGCATTGTACAGGAGAACAACGACCAGGTCTGGAAATTCCAGCGG
TACTTCCTGGTGCAGGAGTACTGCAACCGCCTAAACATCCCCTTCCCCTT
CGTTGTCTTCGCTTATTTCTACATGGTGGTGAAGAAGTGTTTCAATGCTG
CTGTAAAGAGAAGAATATGGAGTCTAATGCCTGCTGTTTCAGAAATGAGG
ACAATGAGACTTTGGCGTGGGAGGGTGTCATGAAGGAGAATTACCTTGTC
AAGATCAACACGAAAGCCAACGACAACTCAGAGGAGATGAGGCATCGGTT
TAGACAACTGGACTCAAAGCTTAACGACCTCAAAAGTCTTCTGAAAGAGA
TTGCTAATAACATCAAGTAA

SEQ ID NO: 5

MSFEGARLSMRSRRNGTMGSTRTLYSSVSRSTDVSYSDSDLVNFIQANFK
KRECVFFTRDSKAMENICKCGYAQSQHIEGTQINQNEKWNYKKHTKEFPT
DAFGDIQFETLGKKGKYLRLSCDTDSETLYELLTQHWHLKTPNLVISVTG
GAKNFALKPRNRKIFSRLIYIAQSKGAWILTGGTHYGLMKYIGEVVRDNT
ISRNSEENIVAIGIAAWGMVSNRDTLIRSCDDEGHFSAQYIMDDFTRDPL
YILDNNHTHLLLVDNGCHGHPTVEAKLRNQLEKYISERTSQDSNYGGKIP
IVCFAQGGGRETLKAINTSVRSKIPCVVVEGSGQIADVIASLVEVEDVLT
SSMVKEKLVRFLPRTVSRLPEEEIESWIKWLKEILESSHLLTVIKMEEAG
DEIVSNAISYALYKAFSTNEQDKDNWNGQLKLLLEWNQLDLASDEIFTND
RRWESADLQEVMFTALIKDRPKFVRLFLENGLNLQKFLTNEVLTELFSTH
PSTLVYRNLQIAKNSYNDALLTFVWKLVANFRRSFWKEDRSSREDLDVEL
HDASLTTRIIPLQALPIWAILQNKKELSKvIWEQTKGCTLAALGASKLLK
TLARVKNDINAAGESEELANEYETRAVELFTECYSNDEDLAEQLLVYSCE
AWGGSNCLELAVEATDQHFIAQPGVQNFLSKQWYGEISRDTKNWFIILCL
FIIPLVGCGLVSFRKKPIDKHKKLLWYYVAFFTSPFVVFSWNVVFYIAFL
LLFAYVLLMDFHSVPHTPELILYALVFVLFCDEVRQWYMNGVNYFTDLWN
VMDTLGLFYFIAGIVFRLNSSNICSSLYSGRVIFCLDYIIFTLRLIHIFT
VSRNLGPKIIMLQRMLIDVFFFLFLFAVWMVAFGVARQGILRQNEQRWRW
IFRSVIYEPYLANFGQVPSDVDSTTYDEISHCTFSGNESKPLCVELDEHN
LPRFPEWITIPLVCIYNLSTNILLVNLLVAMFGYTVGIVQENNDQVWKFQ
RYFLVQEYCNRLIQIPFPFVVFAYFYMVVKKCFKCCCKEKNMESNACCFR
NEDNETLAWEGVMRENYLVKINTKANDNSEEMRHRFRQLDSKLNDLKSLL
KEIAN

SEQ ID NO: 19

```
Genomic Locus from 5′prF to 3′prR
Sequecne Range: 67901 to 74400
>5′prF
    |
    | 68000
    GCATGTACTTGGGGATGTACATAGGATGGGTGGTCCCCAGAAAGGAGTCACATAGGGGTCCTCCAGCGTACTCCTGCTCCCCAGGTGTCTGTGCATAACT
    CGTACATGAACCCCTACATGTATCCTACCCACCAGGGGTCTTTCCTCAGTGTATCCCCAGGAGGTCGCATGAGGACGAGGGGTCCACAGACACGTATTGA
                                                                                                                                                                                              <5′pcR
                                                                                                                                                                                                  |
                                                                                                                                                                                                  68100
    TTCTCCCAAGTATAAGAAATATATTGAAATTTAATTTAAGACGATTTCTAAGCTGTTATCTCACTTGCACAAGGTCTTAGGTTTGATCCCTAGCACTGAA
    AAGAGGGTTCATATTCTTTATATAACTTTAAATTAAATTCTGCTAAAGATTCGACAATAGAGTGAACGTGTTCCAGAATCCAAACTAGGGATCGTGACTT
                                                                                                                                                                                                  68200
    TTATAAGATTAAGATAAAAATAATAATTTCTAGCTTTATTCTGTTATAGACCAGGAGGTGGGGGGGGGAGGTCCTGTAAAAATTCTGCTTTGAAATCTTT
    AATATTCTAATTCTATTTTTATTATTAAAGATCGAAATAAGACAATATCTGGTCCTCCACCCCCCCCCTCCAGGACATTTTTAAGACGAAACTTTAGAAA
                                                                                          >5′armF
                                                                                                |
                                                                                                | 68300
    ATTGAGAATTTTCTTGGTGGTTTACTATGTCACTATTTCTTTAAATGAATCTATGGATACCTGTGCTTCTGTCTCCACTTCAGCCCTTGGCTGCTGGATT
    TAACTCTTAAAAGAACCACCAAATGATACAGTGATAAAGAAATTTACTTAGATACCTATGGACACGAAGACAGAGGTGAAGTCGGGAACCGACGACCTAA
                                                                                                                                                                                                  68400
    CACACAGCCTTAGTTAGATGTCTCAGGCCTTCCGGTGTTCTCATTCTCTTTATCCCTGTATCCTGGATGCTACTGTGTGATAATATTGAGATAGTGGGTC
    GTGTGTCGGAATCAATCTACAGAGTCCGGAAGGCCACAAGAGTAAGAGAAATAGGGACATAGGACCTACGATGACACACTATTATAACTCTATCACCCAG
                                                                                                                                                                                                  68500
    AGCCAGGCCTCCTGCTGTTAGAAGCTTAATACTTTGTAATATTTATCCAAGTATTTTCTTTTTATATGCTGATTCTACATACAAGACCATTGAATGGTTA
    TCGGTCCGGAGGACGACAATCTTCGAATTATGAAACATTATAAATAGGTTCATAAAAGAAAAATATACGACTAAGATGTATGTTCTGGTAACTTACCAAT
                                                                                                                                                                                                  68600
    GTTTATTGAAGTCCGAGTCTGCTCCAAGCTCATTACAGACTTGACTGAATCACCATGGATGGTAGACTTTGCCATCTCCTGCTTGTCTACACTTTTCAGA
    CAAATAACTTCAGGCTCAGACGAGGTTCGAGTAATGTCTGAACTGACTTAGTGGTACCTACCATCTGAAACGGTAGAGGACGAACAGATGTGAAAAGTCT
                                                                                                                                                                                                  68700
    AGTTAACGTTGATGTTAGCACCTCAACAGATTACGAATTAACCAACATCTTCCCACCCTGCCCCCCAAAGACCAAAGGCTGTACTCTGGCAGCCTTGGGG
    TCAATTGCAACTACAATCGTGGAGTTGTCTAATGCTTAATTGGTTGTAGAAGGGTGGGACGGGGGGTTTCTGGTTTCCGACATGAGACCGTCGGAACCCC
                                                                                                                                                                                                  68800
    GCCAGCAAGCTTCTGAAGACCCTGGCCAAAGTTAAGAATGATATCAACGCTGCTGGGGAATCGGAGGAACTGGCCAATGAATATGAGACCCGAGCAGTGG
    CGGTCGTTCGAAGACTTCTGGGACCGGTTTCAATTCTTACTATAGTTGCGACGACCCCTTAGCCTCCTTGACCGGTTACTTATACTCTGGGCTCGTCACC
                                                                                                                                                                                                  68900
    GTGAGCGCACTGTAGTGTATGCCAATCTATCGCACCTAGAAGGCTTGCGGTGGGGGAGGGTAGAAGGAGCTGTTTTAGATAAGGAGAGGTAGAGAGAGGA
    CACTCGCGTGACATCACATACGGTTAGATAGCGTGGATCTTCCGAACGCCACCCCCTCCCATCTTCCTCGACAAAATCTATTCCTCTCCATCTCTCTCCT
                                                                                                                                                                                                  69000
    TCAGAGAGGGAAGGGGCCGTGCCAGGTGTTGAAGGCAGATGAATTCCATAAGCATTTATCAACACCCTCTGCATTAAAGGGCCCCACTGACTATGTGTGT
    AGTCTCTCCCTTCCCCGGCACGGTCCACAACTTCCGTCTACTTAAGGTATTCGTAAATAGTTGTGGGAGACGTAATTTCCCGGGGTGACTGATACACACA
                                                                                                                                                                                                  69100
    CTAAAGAAACACCCATGTAGTGAGGCTTATATTTGGACAGCTTTCTCTATGCTTCTGTCTGCCTGGATCCTCGTGCCCAATCTGTTCAGTTTTTGACTGG
    GATTTCTTTGTGGGTACATCACTCCGAATATAAACCTGTCGAAAGAGATACGAAGACAGACGGACCTAGGAGCACGGGTTAGACAAGTCAAAAACTGACC
                                                                                                                                                                                                  69200
    GGCTAGGCAGGCCACATCTCATGTCTGCCAGCTGAGCCCAGCTCTCCCTACCCTGATGATACAGAGTTGTTCACCGAGTGTTACAGCAATGATGAAGACT
    CCGATCCGTCCGGTGTAGATACAGACGGTCGACTCGGGGTCGAGAGGGATGGGACTACTATGTCTCAACAAGTGGCTCACAATGTCGTTACTACTTCTGA
                                                                                                                                                                                                  69300
    TGGCAGAACAGCTACTGGTCTACTCCTGCGAAGCCTGGGGTGGGAGCAACTGTCTGGAGCTGGCAGTGGAGGCTACAGATCAGCATTTCATCGCTCAGCC
    ACCGTCTTGTCGATGACCAGATGAGGACGCTTCGGACCCCACCCTCGTTGACAGACCTCGACCGTCACCTCCGATGTCTAGTCGTAAAGTAGCGAGTCGG
                                                                                                                                                                                                  69400
    TGGGGTCCAGGTAAGAAAAAGCCAGGACCTGAAAAATGTACCCAGGAATGGGCTATCAGTAGCATTAAGAAAATGACATTAAAAGTCCGCATTTCCCAAC
    ACCCCAGGTCCATTCTTTTTCGGTCCTGGACTTTTTACATGGGTCCTTACCCGATAGTCATCGTAATTCTTTTACTGTAATTTTCAGGCGTAAAGGGTTG
                                                                                                                                                                                                  69500
    CCGTGTAAAAAACATTGAGGGAGTACCTGGGTGAAAGTGTGAAAGACGGTGACACATTAAGCTTTTGTGAAAAGTCTTACCTTAAGAGATGGCAAAGGAA
    GGCACATTTTTTGTAACTCCCTCATGGACCCACTTTCACACTTTCTGCCACTGTGTAATTCGAAAACACTTTTGAGAATGGAATTCTCTACCGTTTCCTT
                                                                                                                                                                                                  69600
    TGCATCAGTTAGCTTTCCATGACTGTAGTAAAATGCCTGAGGTGGTCAACATAAAAAGAGGAAAGGTTTATTTTGACAAACAGTGTTTGGAGTTTTAGTT
    ACGTAGTCAATCGAAAGGTACTGACATCATTTTACGGACTCCACCAGTTGTATTTTTCTCCTTTCCAAATAAAACTGTTTGTCACAAACCTCAAAATCAA
                                                                                                                                                                                                  69700
    TATGGTAGGTTGTGCTCATTGCTTCTGGGTTCATGATGAAGCAGACATTACGGCCAGAGCATGGTAGAGGTAGACATTTACCTCATGACAGCCAGGAAGC
    ATACCATCCAACACGAGTAACGAAGACCCAAGTACTACTTCGTCTGTAATGCCGGTCTCGTACCATCTCCATCTGTAAATGGAGTACTGTCGGTCCTTCG
                                                                                                                                                                                                  69800
    AAATGATGAGAAAGAGGAGGACCCAAAGTCCCTTCAATGATGTTACCCTCAATGGTCTAAAATCTTTCCCCTAGACTTCAACATCTTAAAGACTGCACTA
    TTTACTACTCTTTCTCCTCCTGGGTTTCAGGGAAGTTACTACAATGGGAGTTACCAGATTTTAGAAAGGGGATCTGAAGTTGTAGAATTTCTGACGTGAT
                                                                                                                                                                                                  69900
    CCTTCCAAAGTGTCATACTGGAAACTATGTCTTTCGGGTATGGACCTTCGAAAAATATTTAAGACACAAACCACAGTGTGATGCCTAGCTTTAATTGTAA
    GGAAGGTTTCACAGTATGACCTTTGATACAGAAAGCCCATACCTGGAAGCTTTTTATAAATTCTGTGTTTGGTGTCACACTACGGATCGAAATTAACATT
                                                                                                                                                                                                  70000
    ACTTAATAGATTATAGAATCACCTAGGAAGAGTCTCAGGGAAGGGTTGCCTAGTTCAGTGTCTGTGGGCAGTTTAACTGCCTTGTGGTTGCCTTGATCAG
    TGAATTATCTAATATCTTAGTGGATCCTTCTCAGAGTCCCTTCCCAACGGATCAAGTCACAGACACCCGTCAAATTGACGGAACACCAACGGAACTAGTC
                                                                                                                                                                                                  70100
    CTTCGTTGGTGTGGGAAGAAGACTCAGCCTACTATGGGTGGCACTATTTCCTAGATTTGGACCCTGGACTGTATAAGAGTAGAGAAAGCTAGCTGAACAC
    GAAGCAACCACACCCTTCTTCTGAGTCGGATGATACCCACCGTGATAAAGGATCTAAACCTGGGACCTGACATATTCTCATCTCTTTCGATCGACTTGTG
                                                                                                                                                                                                  70200
    AAAGCATGGAATCACTTCTCTGCTTTTGATTGAGGATATGACATGACTCGCTGCCTCAAGTTCCTGCCTTGATTTCCCCGCTGTGATGGACCATAAGGTG
    TTTCGTACCTTAGTGAAGAGACGAAAACTAACTCCTATACTGTACTGAGCGACGGAGTTCAAGGACGGAACTAAAGGGGCGACACTACCTGGTATTCCAC
                                                                                                                                                                                                  70300
    GAGCTGAGAGATAAGATAACCCTCATCTCCCCAAAGATGCTTTTTGTCAGGATATTTTATCACAGTGACAGAAATGAAACCAAGACACATGGCAAGGAGA
    CTCGACTCTCTATTCTATTGGGAGTAGAGGGGTTTCTACGAAAAACAGTCCTATAAAATAGTGTCACTGTCTTTACTTTGGTTCTGTGTACCGTTCCTCT
                                                                                                                                                                                                  70400
    AAGAATTGTTCGGTCTTCCACAAAGCAAATAGTACATTATAATGCTGGGTATCCTGCCTGGGTCTTAGTCTACTGAAGTAGATGTGAGATGATGAACCTC
    TTCTTAACAAGCCAGAAGGTGTTTCGTTTATCATGTAATATTACGACCCATAGGACGGACCCAGAATCAGATGACTTCATCTACACTCTACTACTTGGAG
                                                                                                                                                                                                  70500
    AGTTTCTCATCTGTGAAGTGGGCATAGTTGTCTATACTAGTTCTCTTTCTGTTTATATTAGTTATCTTAAGTTACCTGACAGAGGCAACATAAGGGAAGA
    TCAAAGAGTAGACACTTCACCCGTATCAACAGATATGATCAAGAGAAAGACAAATATAATCAATAGAATTCAATGGACTGTCTCCGTTGTATTCCCTTCT
                                                                                                                                                                                                  70600
    TAGATGTAGTTGGGCTCACAGTGTTAGCCCAACATGGTAGAGTTCATGGTGGTTACATTTCTCACTCCTGGTGGATGATGAGAAAGCAGAAAATGGGACT
    ATCTACATCAACCCGAGTGTCACAATCGGGTTGTACCATCTCAAGTACCACCAATGTAAAGAGTGAGGACCACCTACTACTCTTTCGTCTTTTACCCTGA
                                                                                                                                                                                                  70700
    AGAAACTGGGTTAGGCTATTACTTTAAGGGGCTCACCCTCAGTGTGCCCACCTCCACTGTCTAGACTGGAGAATCTCCCCAAAACAGCACCACCAGTTAG
    TCTTTGACCCAATCCGATAATGAAATTCCCCGAGTGGGAGTCACACGGGTGGAGGTGACAGATCTGACCTCTTAGAGGGGTTTTGTCGTGGTGGTCAATC
                                                                                                                                                                                                  70800
    GGATGAGGTGTTCACACCCAGGAGCCTGTTCAAGCAGAAATAACAGGGGTTGTCAGAGAGTTGCCATGGCGATGCCATGACATTCTATAAGAAAGGGGAG
    CCTACTCCACAAGTGTGGGTCCTCGGACAAGTTCGTCTTTATTGTCCCCAACAGTCTCTCAACGGTACCGCTACGGTACTGTAAGATATTCTTTCCCCTC
                                                                                                                                                                                                  70900
    GTCAGCAGAGTCAGCTGCTTCCACCACAGCCATTATGAGTGATGGAGAAATCTTGAGATGGGTGAAGCTCTTCCTGAGGCAAGTTTAGCAAGCAGCAGCG
    CAGTCGTCTCAGTCGACGAAGGTGGTGTCGGTAATACTCACTACCTCTTTAGAACTCTACCCACTTCGAGAAGGACTCCGTTCAAATCGTTCGTCGTCGC
                                                                                                                                                                                                  71000
    TATAACCATGTTCACAGTTGCACTGGGATGTTTGTCTAACCAGGTAGCGATGCAGACGAGCCATGCCGGTTATGTGTTTTATCTGCACCGTGTGTGTGGA
    ATATTGGTACAAGTGTCAACGTGACCCTACAAACAGATTGGTCCATCGCTACGTCTGCTCGGTACGGCCAATACACAAAATAGACGTGGCACACACACCT
                                                                                                                                                                                                  71100
    AATGGGCATCATTCATTTTATATCGAGGACTCCGTGTTCAGAGACGTGCTAGAATATTTTCCATGCTTGCTTGCTAGGAAGTTGGAGCACTGGGATTTAA
    TTACCCGTAGTAAGTAAAATATAGCTCCTGAGGCACAAGTCTCTGCACGATCTTATAAAAGGTACGAACGAACGATCCTTCAACCTCGTGACCCTAAATT
                                                                                                                                                                                                  71200
    TCCTCCATGGATTGCCTCCTGCTTCAAACTTTGTACTCGGTGGCCAGTAAAGAGACCTCAGATGAGGAGGCGGTGCTGTAAGCAGTTTGCTCTAGAGCTG
    AGGAGGTACCTAACGGAGGACGAAGTTTGAAACATGAGCCACCGGTCATTTCTCTGGAGTCTACTCCTCCGCCACGACATTCGTCAAACGAGATCTCGAC
                                                                                                                                                                                                  71300
    AGCTGGAGGTGATTAGTGGGCAGTGAGCTGTGGGAGGAAGGAGAAGACATGCTGTGATGAGAGGGGTGGTGTCCCTGGGAGTTCTTGGCAGCCTCCAGGT
    TCGACCTCCACTAATCACCCGTCACTCGACACCCTCCTTCCTCTTCTGTACGACACTACTCTCCCCACCACAGGGACCCTCAAGAACCGTCGGAGGTCCA
                                                                                                                                                                                                  71400
    CCCCTGTAGAGTGTGCCCCAAGCCCCAGGGTCATCATGCTACTTACTGGGTGCTCTTTCCGGGGTCCAGCTTGCCCCTCCTTGGTCAAGCACGCACCTGT
    GGGGACATCTCACACGGGGTTCGGGGTCCCAGTAGTACGATGAATGACCCACAGAAAGGCCCCCAGGTCGAACGGGGAGGAACCAGTTCGTGCGTGGACA
                                                                                                                                                                                                  71500
    GGAGCATCCCCAAGAGGATATAGCAGGACGAGCACACAGGAGAAGCACGTGGCGCTTGCCATCTTTCTAATAGAAGAGGAGATGCAGGTCTGTGGGCATA
    CCTCGTAGGGGTTCTCCTATATCGTCCTGCTCGTGTGTCCTCTTCGTGCACCGCGAACGGTAGAAAGATTATCTTCTCCTCTACGTCCAGACACCCGTAT
                                                                                                                                                                                                  71600
    GCACACCAGTCTCTCAGTCTCTTCATGGACCTCAGCTTTCCCTTACTCTGCCTTCTTCATGCCAACCCCACTTTGGCTGGTGTTTTCCCCAGCTGTGAGG
    CGTGTGGTCAGAGAGTCAGAGAAGTACCTGGAGTCGAAAGGGAATGAGACGGAAGAAGTACGGTTGGGGTGAAACCGACCACAAAAGGGGTCGACACTCC
                                                                                                                                                                                                  71700
    TTTATTATTTTGTTTGTTTGCTTTGGTATAGTCATCTATCTTTTGGTAGCAATGTGATGTTTCACATCTCTGCCATCATGGAAACCCTTTTCTGCCCCCA
    AAATAATAAAACAAACAAACGAAACCATATCAGTAGATAGAAAACCATCGTTACACTACAAAGTGTAGAGACGGTAGTACCTTTGGGAAAAGACGGGGGT
                                                                                                                                                                                                  71800
    CCTTTTGGGGCTGGTAAGGAGAGTCAGCAGTTGTCTTTGTGCACTCCATCTTTGGTTTTACTTCGCCCTTTTTCTTCTGACTTCCTTTTTAGCTTCCAAA
    GGAAAACCCCCGACCATTCCTCTCAGTCGTCAACAGAAACACGTGAGGTAGAACCAAAATGAAGCCGGGAAAAAGAAGACTGAAGGAAAATCGAAGGTTT
                                                                                                                                                                                                  71900
    TAAAAGCACCCCATCTGCCTCCAGCTAGGAGCCCTGGAGACTCAGCAGACTGCACTGGCCCTGTCTTTAAACTTAGTGTCCCCAAAGAGGAGGGGGCTTG
    ATTTTCGTGGGGTAGACGGAGGTCGATCCTCGGGACCTCTGAGTCGTCTGACGTGACCGGGACAGAAATTTGAATCACAGGGGTTTCTCCTCCCCCGAAC
                                                                                                                                                                                                  72000
    AAACATGCTTGTTTAAACTAAGCTTACACAGATAAAAACTATAGTGGTTAAAAATTCATAGGTTTGGTGAATTTTCACTCACATAACCAGCTCTTTTAAA
    TTTTGTACGAACAAATTTGATTCGAATGTGTCTATTTTTGATACACCAATTTTTAAGTATCCAAACCACTTAAAAGTGAGTGTATTGGTCGAGAAAATTT
                                                                                                                                                                                                  72100
    GCACATAGAACTGAACTTAGGGGAAAAAATACTTAAAAATCAAACAGAATTTTAAGTTGTTTCTTCATGCATTAAGATCCAGACAATATCTAAAAGATTG
    CGTGTATCTTGACTTGAATCCCCTTTTTTATGAATTTTTAGTTTGTCTTAAAATTCAACAAAGAAGTACGTAATTCTAGGTCTGTTATAGATTTTCTAAC
                                                                                                                                                                                                  72200
    AAGACGTGGATGTGGGGGTTAGATATTTTGGGAGTGGGCAGGGCTGAGGAGAAGGGCCCTGTCCTGTCCCCCATGTACTTACATCAATAAAGACTTGTGT
    TTCTGCACCTACACCCCCAATCTATAAAACCCTCACCCGTCCCGACTCCTCTTCCCGGGACAGGACAGGGGGTACATGAATGTAGTTATTTCTGAACACA
                                                                                                                            >hetF
                                                                                                                                |
                                                                                                                    <5′armR >TM
                                                                                                                      | | |
                                                                                                                      | | | 72300
    TCCCTGTCTCTGTTTCCTCCTAGAATTTCCTTTCTAAGCAATGGTATGGAGAGATTTCCCGAGACACGAAGAACTGGAAGATTATCCTGTGTCTATTCAT
    AGGGACAGAGACAAAGGAGGATCTTAAAGGAAAGATTCGTTACCATACCTCTCTAAAGGGCTCTGTGCTTCTTGACCTTCTAATAGGACACAGATAAGTA
                                                    N F L S K Q W Y G E I S R D T K N W K I I L C L F I>
                                                  _{—————————————————————————————————————————————————————————————————————————————}>
                                                                                                                                                                                                  72400
    CATCCCCTTAGTGGGCTGTGGCCTCGTATCATTTAGGTGTGGACTGGGGCATCTAATCTTGTGTGTGAGCATCTTTGAGTATTTTTGAGCATTTGTGTAA
    GTAGGGGAATCACCCGACACCGGAGCATAGTAAATCCACACCTGACCCCGTAGATTAGAACACACACTCGTAGAAACTCATAAAAACTCGTAAACACATT
        I P L V G C G L V S F X>
    _{—————————————————————————————————————}>
                                                                                                                      >3′armF <hetR
                                                                                                                            | |
                                                                                                                            | | 72500
    ATGGAGTGTTCACTGTCCCATCTGTGTGCTCAGCCCAGCATCTTTCCTGGCTTTTCCTAGGTAGGGTTTCAAGCAGGTGGTACTGAGGTTGTCTTCAGTA
    TACCTCACAAGTGACACCGTAGACACACGAGTCGGGTCGTAGAAAGGACCGAAAAGGATCCATCCCAAAGTTCGTCCACCATGACTCCAACAGAAGTCAT
                                                                                                                                                                                                  72600
    TATGAGGCTGAATTTGGATTCTCCGATCAGAAAAGGGGGAGGGGGGGCTGAGTGTTGGCTAGGTCAGCCTTGGGATCCAGTTTTGTCCTGTCTGTTCCTT
    ATACTCCGACTTAAACCTAAGAGGCTAGTCTTTTCCCCCTCCCCCCCGACTCACAACCGATCCAGTCGGAACCCTAGGTCAAAACAGGACAGACAAGGAA
                                                                                                                                                                                                  72700
    GTCTATGGTAGGAAGCAAGCCATGGTTCCCCCCACCCCTCCTTTCTCACTCTTTCTGTGTCTCTCTCTGTGTGTGCATGTACAAGTGCATGTGTGAGTGT
    CAGATACCATCCTTCGTTCGGTACCAAGGGGGGGTGGGGAGGAAAGAGTGAGAAAGACACAGAGAGACACACACGTACATGTTCACGTACQACACTCACA
                                                                                                                                                                                                  72800
    GTGAATACAAGTGTGAGTGACTGTGTGTGTGCACATGTATTAGTATGTGTGTCTGTGAGTACATATGTCTGTGTGAGTAGGCATGCATGTATGTGTAAGT
    CACTTAGTGTTCACACTCACTGACACACACACGTGTACATAATCATACACACCACACTCATGTATACAGACACACTCATCCGTACGTACATACACATTCA
                                                                                                                                                                                                  72900
    ATACAAGTATGTGTGTTTGTGATGTGTATTTGTGACCGTGTCAGTGTGTATAAATGTACCTGAGTATGTGAAAGAGTATATGTGTGTGCCAGTGTGTGCG
    TATGTTCATACACACAAACACTACACATAAACACTGGCACAGTCACACATATTTACATGGACTCATACACTTTCTCATATACACACACGGTCACACACGC
                                                                                                                                                                                                  73000
    AGTGAATGTGACTGGTGACACTCCCCTGCCTCTGACTCTGTCTAGGAAGAAACCCATTGACAAGCACAAGAAGCTGCTGTGGTACTATGTGGCCTTCTTC
    TCACTTACACTGACCACTGTGAGGGGACGGAGACTGAGACAGATCCTTCTTTGGGTAACTGTTCGTGTTCTTCGACGACACCATGATACACCGGAAGAAG
                                                                                                                                                                                                  73100
    ACGTCGCCCTTCGTGGTCTTCTCCTGGAACGTGGTCTTCTACATCGCCTTCCTCCTGCTGTTTGCCTATGTGCTGCTCATGGACTTCCACTCAGTGCCAC
    TGCAGCGGGAAGCACCAGAAGAGGACCTTGCACCAGAAGATGTAGCGGAAGGAGGACGACAAAACGGTACACGACGAGTACCTGAAGGTGAGTCACGGTG
                                                                                                                                                                                                  73200
    ACACCCCCCAGCTGATCCTCTACGCCCTGGTCTTCGTCCTCTTCTGTGATGAAGTGAGGCAGGTAGGCAAGTGCAGCGTCGCTCTTTCCAGGGAGATGTG
    TGTGGGGGCTCGACTAGGAGATGCGGGACCAGAAGCAGGAGAAGACACTACTTCACTCCGTCCATCCGTTCACGTCGCAGCGAGAAAGGTCCCTCTACAC
                                                                                                                                                                                                  73300
    GATACATGTCTGCTCATCCCAGACGGACATGATTCCACACAAGGCTGGTCTATGGAGCTGGTTAGTTTACTGGGGTAAATGTCAAGAGAGCATCTGCATC
    CTATGTACAGACGAGTAGGGTCTGCCTGTACTAAGGTGTGTTCCGACCAGATACCTCGACCACTCAAATGACCCCATTACAGTTCDTCTCGTAGACGTAG
                                                                                                                                                                                                  73400
    ATCAAAAGCCCACCCAAACGACTCATGAAAGCTGCGTCACGGGAGCTCAAAGTCCAATCTGTGAGCAAATTTCTCCACTGAAGAAGCCTGTCCCCAGCAA
    TAGTTTTCGGGTGGGTTTGCTGAGTACTTTCGACGCAGTGCCCTCGAGTTTCAGGTTAGACACTCGTTTAAAGAGGTGACTTCTTCGGACAGGGGTCGTT
                                                                                                                                                                                                  73500
    TGGTTGATCATCAGTTTCCTGGGATGTGCATATTTTAGGATCTTTGCTTCACCATTGAATCCAAACTCTACCCCAGCCCTCATAGAGCAGGTGCATCTTA
    ACCAACTAGTAGTCAAAGGACCCTACACGTATAAAATCCTAGAAACGAAGTGGTAACTTAGGTTTGAGATGGGGTCGGGAGTATCTCGTCCACGTAGAAT
                                                                                                                                                                                                  73600
    GTTTGGGTTTTACTGCTGTGAACAGACACCATGACCAAGGCAACTTTTATAAATGACATCATTTAACGGGCTGGCTTACAGGTTTAGAGGTTCAGTCCAT
    CAAACCCAAAATGACGACACTTGTCTGTGGTACTGGTTCCGTTGAAAATATTTACTGTAGTAAATTGCCCGACCGAATGTCCAAATCTCCAAGTCAGGTA
                                                                                                                                                                                                  73700
    TATCGCCAAGGTGGGAAATGGCAGCAACTAGGCATGGTGCAGGAGGATCTGAGAGTTCTACGTCTTCATATGAAGGCTGCTAGTGGAAGGCTGACTCCCA
    ATAGCGGTTCCACCCTTTACCGTCGTTGATCCGTACCACGTCCTCCTAGACTCTCAAGATGCAGAAGTATACTTCCGACGATCACCTTCCGACTGAGGGT
                                                                                                                                                                                                  73800
    AGCAGCTAGAGCAAGGGTATTAAAGCCCACACCCACCAGGCCACACCTACTCCAATAAGACCATACCTCCCAACAGTGCTACTCCCTGGGCCAAGCATTA
    TCGTCGATCTCGTTCCCATAATTTCGGGTGTGGGTGGTCCGGTGTGGATGAGGTTATTCTGGTATGGAGGGTTGTCACGATGAGGGACCCGGTTCGTAAT
                                                                                                                                                                                                  73900
    CAAACCATCACACTGAGGTAGCAGATGTTGAAGAAATGTTTGTTGTTGGTGAAGGAGGTGCTGTCCTGACTCATGGTGGAAAATGTCTCAGCAAGTCCTA
    GTTTGGTAGTGTGACTCCATCGTCTACAACTTCTTTACAAACAACAACCACTTCCTCCACGACAGGACTGAGTACCACCTTTTACAGAGTCGTTCAGGAT
                                                                                                                                                                                                  74000
    TCAGATTTATCTGTCACTCTCTTGTCTCTTCTGGACCCGAACCTTAAAAATATACCCATTTAGCAAGTATTTGTTAGGTATACAACTCATGGAGTATTAC
    AGTCTAAATAGACAGTGAGAGAACAGAGAAGACCTGGGCTTGGAATTTTATATGGGTAAATCGTTCATAAACAATCCATATGTTGAGTACCTCATAAATG
                                                  <3′armR <3′scr
                                                      | |
                                                      | | 74100
    AGATTACAAAGTACAAGGCTCAGGGGTGGAAGAGATTTGAAGGGGAAACATACTGCCTTTGCCCTGACTAGTCTAAGAACACTGAGTTGGCTTATGTTGT
    TCTAATGTTTCATGTTCCAGTCCCCACCTTCTCTAAACTTCCCTTTGTATGACGGAAACGGGACTGATCAGATTCTCTTGTGACTCAACCGAATACAACA
                              >3′prF
                                  |
                                  | 74200
    CATGCCAACTCTCTCTGGTAGATTTTTATGTGCAGTCTCCAGGACTCCTGGATTGCGAGGGATTCTGGGGCCCTCTGTCAATTCTTAGTATGTCTTAATC
    GTACGGTTGAGAGACCATCTAAAAAATACACGTCAGAGGTCCTGDAGGACCTAACGCTCCCTAAGACCCCGGGAGACAGTTAAGAATCATACAGAATTAG
                                                                                                                                                                                                  74300
    AATTAATGATGTCTGCTATGGAAGTGAGGGCTGGGGGGGGGGGGTTGGGTACCTGTGCCCTATATGCCTGTCTTTATGATTAGTAGGTAAGTGGTGTGGA
    TTAATTACTACAGACGATACCTTCACTCCCGACCCCCCCCCCCCAACCCATGGACACGGGATATACGGACAGAAATACTAATCATCCATTCACCACACCT
                <3′prR
                    |
                    | 74400
    AGATGGTGGAACGTCAGCAGAGGTGGAGGAAGAAAACACACAGGATGGCCGGAGTACCAGTGTACTGACTATAGTTCTGGAAGGCAGAAGTCCAACGTGA
    TCTACCACCTTGCAGTCGTCTCCACCTCCTTCTTTTGTGTGTCCTACCGGCCTCATGGTCACATGACTGATATCAAGACCTTCCGTCTTCAGGTTGCACT
```

We claim:

1. A method of identifying a compound capable of binding to TrpM8 polypeptide, the method comprising:
(a) contacting a TrpM8 polypeptide with a candidate compound; and
(b) determining whether the candidate compound binds to the TrpM8 polypeptide;
wherein the compound is suitable for treating or alleviating pain or stress in an individual, wherein the pain or stress is associated with activity of TrpM8.

2. The method of claim 1, wherein the TrpM8 polypeptide comprises an amino acid sequence shown in SEQ ID NO: 3 or SEQ ID NO: 5 or a sequence having at least 90% sequence identity thereto.

3. The method of claim 1, wherein the compound is an agonist or an antagonist of TrpM8 polypeptide.

4. The method of claim 1, wherein the candidate compound is exposed to a cell expressing a TrpM8 polypeptide.

5. The method of claim 4, wherein a change in conductance or intracellular calcium concentration is detected.

6. The method of claim 5, wherein an increase in conductance or intracellular calcium concentration is detected, thereby identifying an agonist of TrpM8.

7. The method of claim 5, wherein a decrease in conductance or intracellular calcium concentration is detected, thereby identifying an antagonist of TrpM8.

8. The method of claim 1, wherein the pain is selected from the group consisting of acute pain, chronic pain, cutaneous pain, somatic pain, visceral pain, referred pain, phantom pain, neuropathic pain, post operative pain, back pain, lower back pain, joint pain, abdominal pain and chest pain.

9. The method of claim 8, wherein the neuropathic pain is selected from the group consisting of trigeminal neuralgia, glossopharyngeal neuralgia, diabetic neuralgia, postherpetic neuralgia and causalgia.

10. The method of claim 1, wherein the pain is associated with injury or disease selected from the group consisting of cancer, inflammatory bowel disease, arthritis, osteoarthritis, rheumatoid arthritis, myocardial ischaemia, neurological disorders, brain tumours, head trauma, traumatic brain injury (TBI), spinal cord trauma, chronic pain syndromes, chronic fatigue syndrome, lupus, sarcoidosis, arachnoiditis, rheumatic disease, musculoskeletal disease, skin disease, diabetes, and fibromyalgia.

11. The method of claim 10, wherein the cancer is selected from the group consisting of breast, prostate, colon, lung, ovarian, and bone cancer.

12. The method of claim 1, wherein the pain is associated with inflammation, thermal hyperalgesia, migraine, headache, spine and peripheral nerve surgery, menstruation or labour.

13. The method of claim 1, wherein the stress is associated with social anxiety, post traumatic stress disorder, phobias, panic disorder, obsessive compulsive disorder, acute stress disorder, separation anxiety disorder, generalised anxiety disorder, major depression, dysthymia, bipolar disorder, seasonal affective disorder, post natal depression, manic depression, or bipolar depression.

14. The method of claim 1, further comprising:
(c) administering the compound capable of binding to TrpM8 polypeptide to an animal that does not express functional TrpM8 polypeptide; and
(d) determining whether the compound produces side effects in the animal.

15. The method of claim 14, wherein the side effects are selected from the group consisting of: changes to disease resistance; altered inflammatory response; altered tumour susceptibility; a change in blood pressure; neovascularization; a change in eating behavior; a change in body weight; a change in bone density; a change in body temperature; a change in insulin secretion; a change in gonadotropin secretion; a change in nasal and/or bronchial secretion; vasoconstriction; loss of memory; anxiety; hyporeflexia; hyperreflexia; and changes in pain or stress responses, compared with an animal that does not express functional TrpM8 polypeptide to which the compound is not administered.

16. A method of identifying a compound for treating or alleviating pain comprising:
(a) administering a candidate compound capable of binding to TrpM8 polypeptide to an animal; and
(b) determining whether the animal exhibits a change in sensitivity to pain;
thereby identifying a compound for treating or alleviating pain.

17. The method of claim 16, wherein the TrpM8 polypeptide comprises an amino acid sequence shown in SEQ ID NO: 3 or SEQ ID NO: 5 or a sequence having at least 90% sequence identity thereto.

18. The method of claim 16, wherein the animal expresses functional Trp8 polypeptide.

19. The method of claim 16, wherein the animal is a wild type animal.

20. The method of claim 16, wherein the animal is a rodent.

21. The method of claim 16, wherein the animal is a mouse.

22. The method of claim 20, wherein sensitivity to pain is measure in a tail-flick test.

23. The method of claim 16, wherein the pain is selected from the group consisting of acute pain, chronic pain, cutaneous pain, somatic pain, visceral pain, referred pain, phantom pain, neuropathic pain, post operative pain, back pain, lower back pain, joint pain, abdominal pain and chest pain.

24. The method of claim 23, wherein the neuropathic pain is selected from the group consisting of trigeminal neuralgia, glossopharyngeal neuralgia, diabetic neuralgia, postherpetic neuralgia and causalgia.

25. The method of claim 16, wherein the pain is associated with injury or disease selected from the group consisting of cancer, inflammatory bowel disease, arthritis, osteoarthritis, rheumatoid arthritis, myocardial ischaemia, neurological disorders, brain tumours, head trauma, traumatic brain injury (TBI), spinal cord trauma, chronic pain syndromes, chronic fatigue syndrome, lupus, sarcoidosis, arachnoiditis, rheumatic disease, musculoskeletal dise 16e, skin disease, diabetes, and fibromyalgia.

26. The method of claim 25, wherein the cancer is selected from the group consisting of breast, prostate, colon, lung, ovarian, and bone cancer.

27. The method of claim 16, wherein the pain is associated with inflammation, thermal hyperalgesia, migraine, headache, spine and peripheral nerve surgery, menstruation or labour.

28. The method of claim 16, further comprising:
(c) administering the compound capable of binding to TrpM8 polypeptide to an animal that does not express functional TrpM8 polypeptide; and
(d) determining whether the compound produces side effects in the animal.

29. The method of claim 28, wherein the side effects are selected from the group consisting of: changes to disease resistance; altered inflammatory response; altered tumour susceptibility; a change in blood pressure; neovascularization; a change in eating behavior; a change in body weight; a change in bone density; a change in body temperature; a change in insulin secretion; a change in gonadotropin secretion; a change in nasal and/or bronchial secretion; vasoconstriction; loss of memory, anxiety; hyporeflexia; hyperreflexia; and changes in pain or stress responses, compared with an animal that does not express functional TrpM8 polypeptide to which the compound is not administered.

30. A method of identifying a compound for treating or alleviating stress comprising:
(a) administering a candidate compound capable of binding to TrpM8 polypeptide to an animal; and
(b) determining whether the animal exhibits a change in stress;
thereby identifying a compound for treating or alleviating stress.

31. The method of claim 30, wherein the TrpM8 polypeptide comprises an amino acid sequence shown in SEQ ID NO: 3 or SEQ ID NO: 5 or a sequence having at least 90% sequence identity thereto.

32. The method of claim 30, wherein the animal expresses functional TrpM8.

33. The method of claim 30, wherein the animal is a wild type animal.

34. The method of claim 30, wherein the animal is a rodent.

35. The method of claim 30, wherein the animal is a mouse.

36. The method of claim 30, wherein the change in stress is measured by blood plasma corticosterone levels.

37. The method of claim 34, wherein the change in stress is measured in an open field test.

38. The method of claim 30, wherein the stress is associated with social anxiety, post traumatic stress disorder, phobias, panic disorder, obsessive compulsive disorder, acute stress disorder, separation anxiety disorder, generalised anxiety disorder, major depression, dysthymia, bipolar disorder, seasonal affective disorder, post natal depression, manic depression, or bipolar depression.

39. The method of claim 30, further comprising:
(c) administering the compound capable of binding to TrpM8 polypeptide to an animal that does not express functional TrpM8 polypeptide; and
(d) determining whether the compound produces side effects in the animal.

40. The method of claim 39, wherein the side effects are selected from the group consisting of: changes to disease resistance; altered inflammatory response; altered tumour susceptibility; a change in blood pressure; neovascularization; a change in eating behavior; a change in body weight; a change in bone density; a change in body temperature; a change in insulin secretion; a change in gonadotropin secretion; a change in nasal and/or bronchial secretion; vasoconstriction; loss of memory; anxiety; hyporeflexia; hyperreflexia; and changes in pain or stress responses, compared with an animal that does not express functional TrpM8 polypeptide to which the compound is not administered.

41. A method of identifying an agonist of TrpM8 polypeptide, the method comprising:
(a) administering a candidate compound capable of binding to TrpM8 polypeptide to an animal; and
(b) determining whether the animal exhibits an increase in sensitivity to pain or stress;
thereby identifying an agonist of TrpM8 polypeptide.

42. A method of identifying an antagonist of TrpM8 polypeptide the method comprising:
(a) administering a candidate compound capable of binding to TrpM8 polypeptide to an animal; and
(b) determining whether the animal exhibits a decrease in sensitivity to pain or stress;
thereby identifying an antagonist of TrpM8 polypeptide.

43. A method of treating an individual suffering from pain or stress, wherein the pain or stress is associated with TrpM8 activity, the method comprising administering an antagonist of TrpM8 to the individual.

44. A method of diagnosing pain or stress in an individual, wherein the pain or stress is associated with TrpM8 activity, the method comprising detecting a change in expression, level or activity of TrpM8 in the individual or in a cell or tissue of the individual.

45. A transgenic non-human mammal comprising a disruption in the endogenous TrpM8 gene, wherein the disruption results in at least one of the following phenotypes, relative to a wild-type animal:
(a) decreased sensitivity to pain;
(b) decreased stress; or
(c) decreased blood plasma corticosterone levels.

46. The transgenic non-human mammal of claim 45, wherein the disruption is a deletion, insertion, or substitution in the endogenous TrpM8 gene.

47. The transgenic non-human mammal of claim 46, wherein the substitution is with at least a portion of a sequence selected from the group consisting of (i) the sequence shown in SEQ ID NO: 1; (ii) the sequence shown in SEQ ID NO: 2; (iii) the sequence shown in SEQ ID NO: 4; and (ii) a sequence that hybridizes to (i), (ii) or (iii) under stringent conditions of hybridization at 65° C. in 4×SSC and washing at 65° C. in 0.1×SSC.

48. The transgenic non-human mammal of claim 45, which is a mouse.

49. The transgenic non-human mammal of claim 48, wherein the endogenous TrpM8 gene comprises the sequence shown in SEQ ID NO: 4.

50. The transgenic non-human mammal of claim 48, wherein sensitivity to pain is measured in a tail-flick test.

51. The transgenic non-human mammal of claim 48, wherein stress is measured in an open field test.

52. A cell or tissue isolated from the transgenic non-human mammal of claim 45.


SEQUENCE LISTING (19 sequences)

SEQ ID NO: 1 - DNA, 3355 bp, Homo sapiens
aagaaaatcctgcttgacaaaaaccgtcacttaggaaaagatgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacgagacccgggctgttgagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagtggtacgtaaatggggtgaattattttactgacctgtggaatgtgatggacacgctggggcttttttacttcatagcaggaattgtatttcggctccactcttctaataaaagctctttgtattctggacgagtcattttctgtctggactacattattttcactctaagattgatccacatttttactgtaagcagaaacttaggacccaagattataatgctgcagaggatgctgatcgatgtgttcttcttcctgttcctctttgcggtgtggatggtggcctttggcgtggccaggcaagggatccttaggcagaatgagcagcgctggaggtggatattccgttcggtcatctacgagccctacctggccatgttcggccaggtgcccagtgacgtggatggtaccacgtatgactttgcccactgcaccttcactgggaatgagtccaagccactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaagcttaatgatctcaagggtcttctgaaagagattgctaataaaatcaaataa

SEQ ID NO: 2 - DNA, 3315 bp, Homo sapiens
atgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacatcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacgagacccgggctgttgagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagtggtacgtaaatggggtgaattattttactgacctgtggaatgtgatggacacgctggggcttttttacttcatagcaggaattgtatttcggctccactcttctaataaaagctctttgtattctggacgagtcattttctgtctggactacattattttcactctaagattgatccacatttttactgtaagcagaaacttaggacccaagattataatgctgcagaggatgctgatcgatgtgttcttcttcctgttcctctttgcggtgtggatggtggcctttggcgtggccaggcaagggatccttaggcagaatgagcagcgctggaggtggatattccgttcggtcatctacgagccctacctggccatgttcggccaggtgcccagtgacgtggatggtaccacgtatgactttgcccactgcaccttcactgggaatgagtccaagccactgtgtgtggagctggatgagcacaacctgccccggttccccgagtggatcaccatccccctggtgtgcatctacatgttatccaccaacatcctgctggtcaacctgctggtcgccatgtttggctacacggtgggcaccgtccaggagaacaatgaccaggtctggaagttccagaggtacttcctggtgcaggagtactgcagccgcctcaatatccccttccccttcatcgtcttcgcttacttctacatggtggtgaagaagtgcttcaagtgttgctgcaaggagaaaaacatggagtcttctgtctgctgtttcaaaaatgaagacaatgagactctggcatgggagggtgtcatgaaggaaaactaccttgtcaagatcaacacaaaagccaacgacacctcagaggaaatgaggcatcgatttagacaactggatacaaagcttaatgatctcaagggtcttctgaaagagattgctaataaaatcaaataa

SEQ ID NO: 3 - protein, 1104 aa, Homo sapiens
MSFRAARLSMRNRRNDTLDSTRTLYSSASRSTDLSYSESDLVNFIQANFKKRECVFFTKDSKATENVCKCGYAQSQHMEGTQINQSEKWNYKKHTKEFPTDAFGDIQFETLGKKGKYIRLSCDTDAEILYELLTQHWHLKTPNLVISVTGGAKNFALKPRMRKIFSRLIYIAQSKGAWILTGGTHYGLMKYIGEVVRDNTISRSSEENIVAIGIAAWGMVSNRDTLIRNCDAEGYFLAQYLMDDFTRDPLYILDNNHTHLLLVDNGCHGHPTVEAKLRNQLEKYISERTIQDSNYGGKIPIVCFAQGGGKETLKAINTSIKNKIPCVVVEGSGQIADVIASLVEVEDALTSSAVKEKLVRFLPRTVSRLPEEETESWIKWLKEILECSHLLTVIKMEEAGDEIVSNAISYALYKAFSTSEQDKDNWNGQLKLLLEWNQLDLANDEIFTNDRRWESADLQEVMFTALIKDRPKFVRLFLENGLNLRKFLTHDVLTELFSNHFSTLVYRNLQIAKNSYNDALLTFVWKLVANFRRGFRKEDRNGRDEMDIELHDVSPITRHPLQALFIWAILQNKKELSKVIWEQTRGCTLAALGASKLLKTLAKVKNDINAAGESEELANEYETRAVELFTECYSSDEDLAEQLLVYSCEAWGGSNCLELAVEATDQHFIAQPGVQNFLSKQWYGEISRDTKNWKIILCLFIIPLVGCGFVSFRKKPVDKHKKLLWYYVAFFTSPFVVFSWNVVFYIAFLLLFAYVLLMDFHSVPHPPELVLYSLVFVLFCDEVRQWYVNGVNYFTDLWNVMDTLGLFYFIAGIVFRLHSSNKSSLYSGRVIFCLDYIIFTLRLIHIFTVSRNLGPKIIMLQRMLIDVFFFLFLFAVWMVAFGVARQGILRQNEQRWRWIFRSVIYEPYLAMFGQVPSDVDGTTYDFAHCTFTGNESKPLCVELDEHNLPRFPEWITIPLVCIYMLSTNILLVNLLVAMFGYTVGTVQENNDQVWKFQRYFLVQEYCSRLNIPFPFIVFAYFYMVVKKCFKCCCKEKNMESSVCCFKNEDNETLAWEGVMKENYLVKINTKANDTSEEMRHRFRQLDTKLNDLKGLLKEIANKIK

SEQ ID NO: 4 - DNA, 6824 bp, Mus musculus
cctggctgtcccggagcttgatacatagaaaagactgacctcagatacacagagatccttctgcttctgtctcccaagtgctgggatcacaggcaagatgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaacctggctgtcccggagcttgatacatagaaaagactgacctcagatacacagagatccttctgcttctgtctcccaagtgctgggatcacaggcaagatgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaa

SEQ ID NO: 5 - protein, 1101 aa, Mus musculus
MSFEGARLSMRSRRNGTMGSTRTLYSSVSRSTDVSYSDSDLVNFIQANFKKRECVFFTRDSKAMENICKCGYAQSQHIEGTQINQNEKWNYKKHTKEFPTDAFGDIQFETLGKKGKYLRLSCDTDSETLYELLTQHWHLKTPNLVISVTGGAKNFALKPRMRKIFSRLIYIAQSKGAWILTGGTHYGLMKYIGEVVRDNTISRNSEENIVAIGIAAWGMVSNRDTLIRSCDDEGHFSAQYIMDDFTRDPLYILDNNHTHLLLVDNGCHGHPTVEAKLRNQLEKYISERTSQDSNYGGKIPIVCFAQGGGRETLKAINTSVKSKIPCVVVEGSGQIADVIASLVEVEDVLTSSMVKEKLVRFLPRTVSRLPEEEIESWIKWLKEILESSHLLTVIKMEEAGDEIVSNAISYALYKAFSTNEQDKDNWNGQLKLLLEWNQLDLASDEIFTNDRRWESADLQEVMFTALIKDRPKFVRLFLENGLNLQKFLTNEVLTELFSTHFSTLVYRNLQIAKNSYNDALLTFVWKLVANFRRSFWKEDRSSREDLDVELHDASLTTRHPLQALFIWAILQNKKELSKVIWEQTKGCTLAALGASKLLKTLAKVKNDINAAGESEELANEYETRAVELFTECYSNDEDLAEQLLVYSCEAWGGSNCLELAVEATDQHFIAQPGVQNFLSKQWYGEISRDTKNWKIILCLFIIPLVGCGLVSFRKKPIDKHKKLLWYYVAFFTSPFVVFSWNVVFYIAFLLLFAYVLLMDFHSVPHTPELILYALVFVLFCDEVRQWYMNGVNYFTDLWNVMDTLGLFYFIAGIVFRLHSSNKSSLYSGRVIFCLDYIIFTLRLIHIFTVSRNLGPKIIMLQRMLIDVFFFLFLFAVWMVAFGVARQGILRQNEQRWRWIFRSVIYEPYLAMFGQVPSDVDSTTYDFSHCTFSGNESKPLCVELDEHNLPRFPEWITIPLVCIYMLSTNILLVNLLVAMFGYTVGIVQENNDQVWKFQRYFLVQEYCNRLNIPFPFVVFAYFYMVVKKCFKCCCKEKNMESNACCFRNEDNETLAWEGVMKENYLVKINTKANDNSEEMRHRFRQLDSKLNDLKSLLKEIAN

SEQ ID NO: 6 - DNA, 27 bp, Artificial Sequence
  Oligonucleotide primer
ggctgtgtccctgtttgcatgtacttg

SEQ ID NO: 7 - DNA, 27 bp, Artificial Sequence
  Oligonucleotide primer
gtgctagggatcaaacctaagaccttg

SEQ ID NO: 8 - DNA, 36 bp, Artificial Sequence
  Oligonucleotide primer
tttaccggtgaatctatggatacctgtgcttctgtc

SEQ ID NO: 9 - DNA, 38 bp, Artificial Sequence
  Oligonucleotide primer
aaagcggccgcgggaaatctctccataccattgcttag

SEQ ID NO: 10 - DNA, 38 bp, Artificial Sequence
  Oligonucleotide primer
aaaggcgcgccgtagggtttcaagcaggtggtactgag

SEQ ID NO: 11 - DNA, 36 bp, Artificial Sequence
  Oligonucleotide primer
tttggccggcccctgagccttgtactttgtaatctg

SEQ ID NO: 12 - DNA, 27 bp, Artificial Sequence
  Oligonucleotide primer
aggcagtatgtttccccttcaaatctc

SEQ ID NO: 13 - DNA, 27 bp, Artificial Sequence
  Oligonucleotide primer
tggtagatttttatgtgcagtctccag

SEQ ID NO: 14 - DNA, 27 bp, Artificial Sequence
  Oligonucleotide primer
ccaccatcttccacaccacttacctac

SEQ ID NO: 15 - DNA, 27 bp, Artificial Sequence
  Oligonucleotide primer
gacacgaagaactggaagattatcctg

SEQ ID NO: 16 - DNA, 27 bp, Artificial Sequence
  Oligonucleotide primer
acaacctcagtaccacctgcttgaaac

SEQ ID NO: 17 - DNA, 27 bp, Artificial Sequence
  Oligonucleotide primer
cgcatcgccttctatcgccttcttgac

SEQ ID NO: 18 - DNA, 27 bp, Artificial Sequence
  Oligonucleotide primer
aatggccgcttttctggattcatcgac

SEQ ID NO: 19 - DNA, 12999 bp, Artificial Sequence
  Knockout plasmid construct
gcatgtacttggggatgtacataggatgggtggtccccagaaaggagtcacataggggtcctccagcgtactcctgctccccaggtgtctgtgcataactcgtacatgaacccctacatgtatcctacccaccaggggtctttcctcagtgtatccccaggaggtcgcatgaggacgaggggtccacagacacgtattgattctcccaagtataagaaatatattgaaatttaatttaagacgatttctaagctgttatctcacttgcacaaggtcttaggtttgatccctagcactgaaaagagggttcatattctttatataactttaaattaaattctgctaaagattcgacaatagagtgaacgtgttccagaatccaaactagggatcgtgacttttataagattaagataaaaataataatttctagctttattctgttatagaccaggaggtgggggggggaggtcctgtaaaaattctgctttgaaatctttaatattctaattctatttttattattaaagatcgaaataagacaatatctggtcctccaccccccccctccaggacatttttaagacgaaactttagaaaattgagaattttcttggtggtttactatgtcactatttctttaaatgaatctatggatacctgtgcttctgtctccacttcagcccttggctgctggatttaactcttaaaagaaccaccaaatgatacagtgataaagaaatttacttagatacctatggacacgaagacagaggtgaagtcgggaaccgacgacctaacacacagccttagttagatgtctcaggccttccggtgttctcattctctttatccctgtatcctggatgctactgtgtgataatattgagatagtgggtcgtgtgtcggaatcaatctacagagtccggaaggccacaagagtaagagaaatagggacataggacctacgatgacacactattataactctatcacccagagccaggcctcctgctgttagaagcttaatactttgtaatatttatccaagtattttctttttatatgctgattctacatacaagaccattgaatggttatcggtccggaggacgacaatcttcgaattatgaaacattataaataggttcataaaagaaaaatatacgactaagatgtatgttctggtaacttaccaatgtttattgaagtccgagtctgctccaagctcattacagacttgactgaatcaccatggatggtagactttgccatctcctgcttgtctacacttttcagacaaataacttcaggctcagacgaggttcgagtaatgtctgaactgacttagtggtacctaccatctgaaacggtagaggacgaacagatgtgaaaagtctagttaacgttgatgttagcacctcaacagattacgaattaaccaacatcttcccaccctgccccccaaagaccaaaggctgtactctggcagccttggggtcaattgcaactacaatcgtggagttgtctaatgcttaattggttgtagaagggtgggacggggggtttctggtttccgacatgagaccgtcggaaccccgccagcaagcttctgaagaccctggccaaagttaagaatgatatcaacgctgctggggaatcggaggaactggccaatgaatatgagacccgagcagtggcggtcgttcgaagacttctgggaccggtttcaattcttactatagttgcgacgaccccttagcctccttgaccggttacttatactctgggctcgtcaccgtgagcgcactgtagtgtatgccaatctatcgcacctagaaggcttgcggtgggggagggtagaaggagctgttttagataaggagaggtagagagaggacactcgcgtgacatcacatacggttagatagcgtggatcttccgaacgccaccccctcccatcttcctcgacaaaatctattcctctccatctctctccttcagagagggaaggggccgtgccaggtgttgaaggcagatgaattccataagcatttatcaacaccctctgcattaaagggccccactgactatgtgtgtagtctctcccttccccggcacggtccacaacttccgtctacttaaggtattcgtaaatagttgtgggagacgtaatttcccggggtgactgatacacacactaaagaaacacccatgtagtgaggcttatatttggacagctttctctatgcttctgtctgcctggatcctcgtgcccaatctgttcagtttttgactgggatttctttgtgggtacatcactccgaatataaacctgtcgaaagagatacgaagacagacggacctaggagcacgggttagacaagtcaaaaactgaccggctaggcaggccacatctcatgtctgccagctgagcccagctctccctaccctgatgatacagagttgttcaccgagtgttacagcaatgatgaagactccgatccgtccggtgtagagtacagacggtcgactcgggtcgagagggatgggactactatgtctcaacaagtggctcacaatgtcgttactacttctgatggcagaacagctactggtctactcctgcgaagcctggggtgggagcaactgtctggagctggcagtggaggctacagatcagcatttcatcgctcagccaccgtcttgtcgatgaccagatgaggacgcttcggaccccaccctcgttgacagacctcgaccgtcacctccgatgtctagtcgtaaagtagcgagtcggtggggtccaggtaagaaaaagccaggacctgaaaaatgtacccaggaatgggctatcagtagcattaagaaaatgacattaaaagtccgcatttcccaacaccccaggtccattctttttcggtcctggactttttacatgggtccttacccgatagtcatcgtaattcttttactgtaattttcaggcgtaaagggttgccgtgtaaaaaacattgagggagtacctgggtgaaagtgtgaaagacggtgacacattaagcttttgtgaaaagtcttaccttaagagatggcaaaggaaggcacattttttgtaactccctcatggacccactttcacactttctgccactgtgtaattcgaaaacacttttcagaatggaattctctaccgtttcctttgcatcagttagctttccatgactgtagtaaaatgcctgaggtggtcaacataaaaagaggaaaggtttattttgacaaacagtgtttggagttttagttacgtagtcaatcgaaaggtactgacatcattttacggactccaccagttgtatttttctcctttccaaataaaactgtttgtcacaaacctcaaaatcaatatggtaggttgtgctcattgcttctgggttcatgatgaagcagacattacggccagagcatggtagaggtagacatttacctcatgacagccaggaagcataccatccaacacgagtaacgaagacccaagtactacttcgtctgtaatgccggtctcgtaccatctccatctgtaaatggagtactgtcggtccttcgaaatgatgagaaagaggaggacccaaagtcccttcaatgatgttaccctcaatggtctaaaatctttcccctagacttcaacatcttaaagactgcactatttactactctttctcctcctgggtttcagggaagttactacaatgggagttaccagattttagaaaggggatctgaagttgtagaatttctgacgtgatccttccaaagtgtcatactggaaactatgtctttcgggtatggaccttcgaaaaatatttaagacacaaaccacagtgtgatgcctagctttaattgtaaggaaggtttcacagtatgacctttgatacagaaagcccatacctggaagctttttataaattctgtgtttggtgtcacactacggatcgaaattaacattacttaatagattatagaatcacctaggaagagtctcagggaagggttgcctagttcagtgtctgtgggcagtttaactgccttgtggttgccttgatcagtgaattatctaatatcttagtggatccttctcagagtcccttcccaacggatcaagtcacagacacccgtcaaattgacggaacaccaacggaactagtccttcgttggtgtgggaagaagactcagcctactatgggtggcactatttcctagatttggaccctggactgtataagagtagagaaagctagctgaacacgaagcaaccacacccttcttctgagtcggatgatacccaccgtgataaaggatctaaacctgggacctgacatattctcatctctttcgatcgacttgtgaaagcatggaatcacttctctgcttttgattgaggatatgacatgactcgctgcctcaagttcctgccttgatttccccgctgtgatggaccataaggtgtttcgtaccttagtgaagagacgaaaactaactcctatactgtactgagcgacggagttcaaggacggaactaaaggggcgacactacctggtattccacgagctgagagataagataaccctcatctccccaaagatgctttttgtcaggatattttatcacagtgacagaaatgaaaccaagacacatggcaaggagactcgactctctattctattgggagtagaggggtttctacgaaaaacagtcctataaaatagtgtcactgtctttactttggttctgtgtaccgttcctctaagaattgttcggtcttccacaaagcaaatagtacattataatgctgggtatcctgcctgggtcttagtctactgaagtagatgtgagatgatgaacctcttcttaacaagccagaaggtgtttcgtttatcatgtaatattacgacccataggacggacccagaatcagatgacttcatctacactctactacttggagagtttctcatctgtgaagtgggcatagttgtctatactagttctctttctgtttatattagttatcttaagttacctgacagaggcaacataagggaagatcaaagagtagacacttcacccgtatcaacagatatgatcaagagaaagacaaatataatcaatagaattcaatggactgtctccgttgtattcccttcttagatgtagttgggctcacagtgttagcccaacatggtagagttcatggtggttacatttctcactcctggtggatgatgagaaagcagaaaatgggactatctacatcaacccgagtgtcacaatcgggttgtaccatctcaagtaccaccaatgtaaagagtgaggaccacctactactctttcgtcttttaccctgaagaaactgggttaggctattactttaaggggctcaccctcagtgtgcccacctccactgtctagactggagaatctccccaaaacagcaccaccagttagtctttgacccaatccgataatgaaattccccgagtgggagtcacacgggtggaggtgacagatctgacctcttagaggggttttgtcgtggtggtcaatcggatgaggtgttcacacccaggagcctgttcaagcagaaataacaggggttgtcagagagttgccatggcgatgccatgacattctataagaaaggggagcctactccacaagtgtgggtcctcggacaagttcgtctttattgtccccaacagtctctcaacggtaccgctacggtactgtaagatattctttcccctcgtcagcagagtcagctgcttccaccacagccattatgagtgatggagaaatcttgagatgggtgaagctcttcctgaggcaagtttagcaagcagcagcgcagtcgtctcagtcgacgaaggtggtgtcggtaatactcactacctctttagaactctacccacttcgagaaggactccgttcaaatcgttcgtcgtcgctataaccatgttcacagttgcactgggatgtttgtctaaccaggtagcgatgcagacgagccatgccggttatgtgttttatctgcaccgtgtgtgtggaatattggtacaagtgtcaacgtgaccctacaaacagattggtccatcgctacgtctgctcggtacggccaatacacaaaatagacgtggcacacacacctaatgggcatcattcattttatatcgaggactccgtgttcagagacgtgctagaatattttccatgcttgcttgctaggaagttggagcactgggatttaattacccgtagtaagtaaaatatagctcctgaggcacaagtctctgcacgatcttataaaaggtacgaacgaacgatccttcaacctcgtgaccctaaatttcctccatggattgcctcctgcttcaaactttgtactcggtggccagtaaagagacctcagatgaggaggcggtgctgtaagcagtttgctctagagctgaggaggtacctaacggaggacgaagtttgaaacatgagccaccggtcatttctctggagtctactcctccgccacgacattcgtcaaacgagatctcgacagctggaggtgattagtgggcagtgagctgtgggaggaaggagaagacatgctgtgatgagaggggtggtgtccctgggagttcttggcagcctccaggttcgacctccactaatcacccgtcactcgacaccctccttcctcttctgtacgacactactctccccaccacagggaccctcaagaaccgtcggaggtccacccctgtagagtgtgccccaagccccagggtcatcatgctacttactgggtgctctttccggggtccagcttgcccctccttggtcaagcacgcacctgtggggacatctcacacggggttcggggtcccagtagtacgatgaatgacccacgagaaaggccccaggtcgaacggggaggaaccagttcgtgcgtggacaggagcatccccaagaggatatagcaggacgagcacacaggagaagcacgtggcgcttgccatctttctaatagaagaggagatgcaggtctgtgggcatacctcgtaggggttctcctatatcgtcctgctcgtgtgtcctcttcgtgcaccgcgaacggtagaaagattatcttctcctctacgtccagacacccgtatgcacaccagtctctcagtctcttcatggacctcagctttcccttactctgccttcttcatgccaaccccactttggctggtgttttccccagctgtgaggcgtgtggtcagagagtcagagaagtacctggagtcgaaagggaatgagacggaagaagtacggttggggtgaaaccgaccacaaaaggggtcgacactcctttattattttgtttgtttgctttggtatagtcatctatcttttggtagcaatgtgatgtttcacatctctgccatcatggaaacccttttctgcccccaaaataataaaacaaacaaacgaaaccatatcagtagatagaaaaccatcgttacactacaaagtgtagagacggtagtacctttgggaaaagacgggggtccttttgggggctggtaaggagagtcagcagttgtctttgtgcactccatcttggttttacttcggccctttttcttctgacttccttttagcttccaaaggaaaacccccgaccattcctctcagtcgtcaacagaaacacgtgaggtagaaccaaaatgaagccgggaaaaagaagactgaaggaaaatcgaaggttttaaaagcaccccatctgcctccagctaggagccctggagactcagcagactgcactggccctgtctttaaacttagtgtccccaaagaggagggggcttgattttcgtggggtagacggaggtcgatcctcgggacctctgagtcgtctgacgtgaccgggacagaaatttgaatcacaggggtttctcctcccccgaacaaacatgcttgtttaaactaagcttacacagataaaaactatagtggttaaaaattcataggtttggtgaattttcactcacataaccagctcttttaaatttgtacgaacaaatttgattcgaatgtgtctatttttgatatcaccaatttttaagtatccaaaccacttaaaagtgagtgtattggtcgagaaaatttgcacatagaactgaacttaggggaaaaaatacttaaaaatcaaacagaattttaagttgtttcttcatgcattaagatccagacaatatctaaaagattgcgtgtatcttgacttgaatccccttttttatgaatttttagtttgtcttaaaattcaacaaagaagtacgtaattctaggtctgttatagattttctaacaagacgtggatgtgggggttagatattttgggagtgggcagggctgaggagaagggccctgtcctgtcccccatgtacttacatcaataaagacttgtgtttctgcacctacacccccaatctataaaaccctcacccgtcccgactcctcttcccgggacaggacagggggtacatgaatgtagttatttctgaacacatccctgtctctgtttcctcctagaatttcctttctaagcaatggtatggagagatttcccgagacacgaagaactggaagattatcctgtgtctattcatagggacagagacaaaggaggatcttaaaggaaagattcgttaccatacctctctaaagggctctgtgcttcttgaccttctaataggacacagataagtacatccccttagtgggctgtggcctcgtatcatttaggtgtggactggggcatctaatcttgtgtgtgagcatctttgagtatttttgagcatttgtgtaagtaggggaatcacccgacaccggagcatagtaaatccacacctgaccccgtagattagaacacacactcgtagaaactcataaaaactcgtaaacacattatggagtgttcactgtggcatctgtgtgctcagcccagcatctttcctggcttttcctaggtagggtttcaagcaggtggtactgaggttgtcttcagtatacctcacaagtgacaccgtagacacacgagtcgggtcgtagaaaggaccgaaaaggatccatcccaaagttcgtccaccatgactccaacagaagtcattatgaggctgaatttggattctccgatcagaaaagggggagggggggctgagtgttggctaggtcagccttgggatccagttttgtcctgtctgttccttatactccgacttaaacctaagaggctagtcttttccccctcccccccgactcacaaccgatccagtcggaaccctaggtcaaaacaggacagacaaggaagtctatggtaggaagcaagccatggttccccccacccctcctttctcactctttctgtgtctctctctgtgtgtgcatgtacaagtgcatgtgtgagtgtcagataccatccttcgttcggtaccaaggggggtggggaggaaagagtgagaaagacacagagagagacacacacgtacatgttcacgtacacactcacagtgaatacaagtgtgagtgactgtgtgtgtgcacatgtattagtatgtgtgtctgtgagtacatatgtctgtgtgagtaggcatgcatgtatgtgtaagtcacttatgttcacactcactgacacacacacgtgtacataatcatacacacagacactcatgtatacagacacactcatccgtacgtacatacacattcaatacaagtatgtgtgtttgtgatgtgtatttgtgaccgtgtcagtgtgtataaatgtacctgagtatgtgaaagagtatatgtgtgtgccagtgtgtgcgtatgttcatacacacaaacactacacataaacactggcacagtcacacatatttacatggactcatacactttctcatatacacacacggtcacacacgcagtgaatgtgactggtgacactcccctgcctctgactctgtctaggaagaaacccattgacaagcacaagaagctgctgtggtactatgtggccttcttctcacttacactgaccactgtgaggggacggagactgagacagatccttctttgggtaactgttcgtgttcttcgacgacaccatgatacaccggaagaagacgtcgcccttcgtggtcttctcctggaacgtggtcttctacatcgccttcctcctgctgtttgcctatgtgctgctcatggacttccactcagtgccactgcagcgggaagcaccagaagaggaccttgcaccagaagatgtagcggaaggaggacgacaaacggatacacgacgagtacctgaaggtgagtcacggtgacacccccgagctgatcctctacgccctggtcttcgtcctcttctgtgatgaagtgaggcaggtaggcaagtgcagcgtcgctctttccagggagatgtgtgtgggggctcgactaggagatgcgggaccagaagcaggagaagacactacttcactccgtccatccgttcacgtcgcagcgagaaaggtccctctacacgatacatgtctgctcatcccagacggacatgattccacacaaggctggtctatggagctggtgagtttactggggtaaatgtcaagagagcatctgcatcctatgtacagacgagtagggtctgcctgtactaaggtgtgttccgaccagatacctcgaccactcaaatgaccccatttacagttctctcgtagacgtagatcaaaagcccacccaaacgactcatgaaagctgcgtcacgggagctcaaagtccaatctgtgagcaaatttctccactgaagaagcctgtccccagcaatagttttcgggtgggtttgctgagtactttcgacgcagtgccctcgagtttcaggttagacactcgtttaaagaggtgacttcttcggacaggggtcgtttggttgatcatcagtttcctgggatgtgcatattttaggatctttgcttcaccattgaatccaaactctaccccagccctcatagagcaggtgcatcttaaccaactagtagtcaaaggaccctacacgtataaaatcctagaaacgaagtggtaacttaggtttgagatggggtcgggagtatctcgtccacgtagaatgtttgggttttactgctgtgaacagacaccatgaccaaggcaacttttataaatgacatcatttaacgggctggcttacaggtttagaggttcagtccatcaaacccaaaatgacgacacttgtctgtggtactggttccgttgaaaatatttactgtagtaaattgcccgaccgaatgtccaaatctccaagtcaggtatatcgccaaggtgggaaatggcagcaactaggcatggtgcaggaggatctgagagttctacgtcttcatatgaaggctgctagtggaaggctgactcccaatagcggttccaccctttaccgtcgttgatccgtaccacgtcctcctagactctcaagatgcagaagtatacttccgacgatcaccttccgactgagggtagcagctagagcaagggtattaaagcccacacccaccaggccacacctactccaataagaccatacctcccaacagtgctactccctgggccaagcattatcgtcgatctcgttcccataatttcgggtgtgggtggtccggtgtggatgaggttattctggtatggagggttgtcacgatgagggacccggttcgtaatcaaaccatcacactgaggtagcagatgttgaagaaatgtttgttgttggtgaaggaggtgctgtcctgactcatggtggaaaatgtctcagcaagtcctagtttggtagtgtgactccatcgtctacaacttctttacaaacaacaaccacttcctccacgacaggactgagtaccaccttttacagagtcgttcaggattcagatttatctgtcactctcttgtctcttctggacccgaaccttaaaatatacccatttagcaagtatttgttaggtatacaactcatggagtatttacagtctaaatagacagtgagagaacagagaagacctgggcttggaattttatatgggtaaatcgttcataaacaatccatatgttgagtacctcataaatgagattacaaagtacaaggctcaggggtggaagagatttgaaggggaaacatactgcctttgccctgactagtctaagaacactgagttggcttatgttgttctaatgtttcatgttccgagtccccaccttctctaaacttcccctttgtatgacggaaacgggactgatcagattcttgtgactcaaccgaatacaacacatgccaactctctctggtagatttttatgtgcagtctccaggactcctggattgcgagggattctggggccctctgtcaattcttagtatgtcttaatcgtacggttgagagagaccatctaaaaatacacgtcagaggtcctgaggacctaacgctccctaagaccccgggagacagttaagaatcatacagaattagaattaatgatgtctgctatggaagtgagggctggggggggggggttgggtacctgtgccctatatgcctgtctttatgattagtaggtaagtggtgtggattaattactacagacgataccttcactcccgaccccccccccccaacccatggacacgggatatacggacagaaatactaatcatccattcaccacacctagatggtggaacgtcagcagaggtggaggaagaaaacacacaggatggccggagtaccagtgtactgactatagttctggaaggcagaagtccaacgtgatctaccaccttgcagtcgtctccacctccttcttttgtgtgtcctaccggcctcatggtcacatgactgatatcaagaccttccgtcttcaggttgcac